(19) United States
(12) Patent Application Publication
(10) Pub. No.: US 2026/0327205 A1
(43) Pub. Date: Oct. 22, 2026

(54) C-LINKED PYRROLIDINE-INDOLE COMPOUNDS AND COMPOSITIONS COMPRISING THE SAME

(71) Applicant: Compass Pathfinder Limited, Altrincham (GB)

(72) Inventors: John Richard MORPHY, Altrincham (GB); Thomas Matthew BAKER, Nottingham (GB); Mark PICHOWICZ, Nottingham (GB); James PICKLES, Nottingham (GB); Andrew William BROWN, Nottingham (GB)

(73) Assignee: Compass Pathfinder Limited, Altrincham (GB)

(21) Appl. No.: 19/644,323

(22) Filed: Apr. 10, 2026

Related U.S. Application Data
(60) Provisional application No. 63/787,354, filed on Apr. 11, 2025.

(51) Int. Cl. C07D 401/14; A61K 31/404; A61K 31/407; A61K 31/4155; A61K 31/4196; A61K 31/422; A61K 31/4439; C07D 403/06; C07D 403/14; C07D 405/14; C07D 409/14; C07D 413/14; C07D 491/056

(52) U.S. Cl. CPC C07D 401/14; A61K 31/404; A61K 31/407; A61K 31/4155; A61K 31/4196; A61K 31/422; A61K 31/4439; C07D 403/06; C07D 403/14; C07D 405/14; C07D 409/14; C07D 413/14; C07D 491/056

(57) ABSTRACT

Provided herein are compounds of formula (I), or pharmaceutically acceptable salts or deuterated forms thereof, wherein R¹, R², R³, R⁴, n, and m are defined herein. Also provided herein are pharmaceutical compositions comprising a compound of formula (I) or pharmaceutically acceptable salt or deuterated form thereof, and methods of using a compound of formula (I) or pharmaceutically acceptable salt or deuterated form thereof, e.g., in the treatment of the treatment of a serotonin 5-HT2A receptor associated disease/disorder.

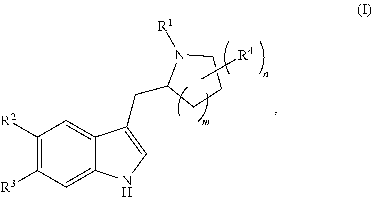

### CROSS-REFERENCE TO RELATED APPLICATIONS

The present application claims priority to U.S. Provisional Patent Application No. 63/787,354 filed Apr. 11, 2025, which is hereby incorporated by reference in its entirety.

### BACKGROUND

Over 50% of adults in the United States will be diagnosed with a psychiatric disorder at some point in their lifetime. Nearly 1 in 5 suffer from mental illness, and nearly 1 in 25 are afflicted with severe mental illness, such as major depression, schizophrenia, or bipolar disorder.

Psychedelics show promising activity in treating mental illness. New psychedelic compounds are needed for treating mental illness.

### SUMMARY

In embodiments, the present disclosure provides a compound of formula (I):

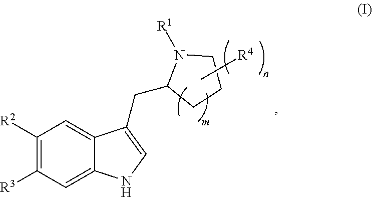

or a pharmaceutically acceptable salt, deuterated form or stereoisomer thereof
wherein:
R¹ is —H, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl; wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more -halo, oxo, —OH, —NH₂, —CN, —O—C₁₋₆ alkyl, or —C₁₋₆ alkyl;
R² is -halo, —O—C₁₋₆ alkyl, —S—C₁₋₆ alkyl, —S(O)₂—C₁₋₆ alkyl, —SH, —NH—C₁₋₆ alkyl, —N(C₁₋₆ alkyl)₂, —NH₂, —CN, —O(C═O)—C₁₋₆ alkyl, or —NH(C═O)—C₁₋₆ alkyl;
R³ is -halo, —O—C₁₋₆ alkyl, —OH, —S—C₁₋₆ alkyl, —S(O)₂—C₁₋₆ alkyl, —SH, —NH—C₁₋₆ alkyl, —N(C₁₋₆ alkyl)₂, —NH₂, —CN, —O(C═O)—C₁₋₆ alkyl, or —NH(C═O)—C₁₋₆ alkyl; or
R² and R³ together with the carbon to which they are attached from a heterocyclyl;
R⁴ is —C₁₋₆ alkyl, -aryl, -heteroaryl, —C₁₋₆ alkylene-aryl, —C₁₋₆ alkylene-heteroaryl, —C₁₋₆ alkylene-OH, —C₁₋₆ alkylene-O—C₁₋₆ alkyl, or —O—C₁₋₆ alkyl;
n is 0, 1, 2, or 3; and
m is 0 or 1,
and wherein, the compound of Formula (I) is not:

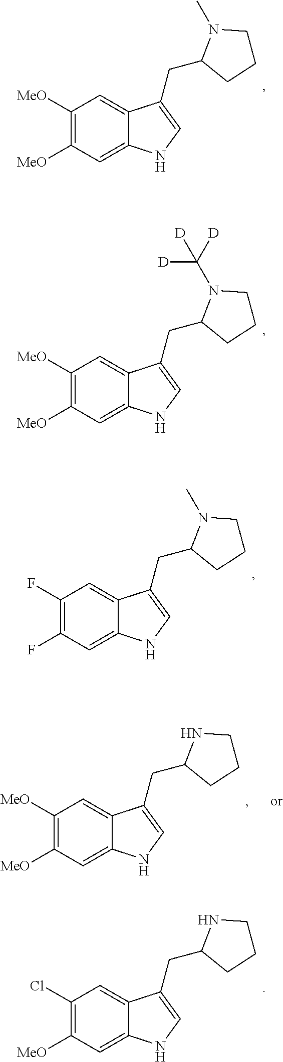 [SMILES: COc1cc2[nH]cc(CC3CCCN3C)c2cc1OC ; [2H]C([2H])([2H])N1CCCC1Cc1c[nH]c2cc(OC)c(OC)cc12 ; CN1CCCC1Cc1c[nH]c2cc(F)c(F)cc12 ; COc1cc2[nH]cc(CC3CCCN3)c2cc1OC ; COc1cc2[nH]cc(CC3CCCN3)c2cc1Cl]

In embodiments, the compound is selected from any one of the compounds in Table A or Table B.

### DETAILED DESCRIPTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

### Definitions

Listed below are definitions of various terms used in the specification and claims to describe the present disclosure.

Unless defined otherwise, all technical and scientific terms used in this disclosure have the same meanings as commonly understood by one of ordinary skill in the art to which this disclosure belongs.

The terms below, as used herein, have the following meanings, unless indicated otherwise:

“Alkyl” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl.

“Alkylene” refers to a fully saturated, straight or branched divalent hydrocarbon chain radical, and having from one to twelve carbon atoms. Non-limiting examples of C₁-C₁₂ alkylene include methylene, ethylene, propylene, n-butylene, ethenylene, propenylene, n-butenylene, propynylene, n-butynylene, and the like. The alkylene is attached to the rest of the molecule through a single bond and to the radical group through a single bond. The points of attachment of the alkylene to the rest of the molecule and to the radical group can be through one carbon or any two carbons within the chain.

“Alkenyl” refers to a straight or branched hydrocarbon chain radical having from two to twelve carbon atoms, and having one or more carbon-carbon double bonds. Each alkenyl is attached to the rest of the molecule by a single bond. Alkenyl comprising any number of carbon atoms from 2 to 12 are included. An alkenyl comprising up to 12 carbon atoms is a C₂-C₁₂ alkenyl, an alkenyl comprising up to 10 carbon atoms is a C₂-C₁₀ alkenyl, an alkenyl comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl.

“Alkynyl” refers to a straight or branched hydrocarbon chain radical having from two to twelve carbon atoms, and having one or more carbon-carbon triple bonds. Each alkynyl is attached to the rest of the molecule by a single bond. Alkynyl comprising any number of carbon atoms from 2 to 12 are included. An alkynyl comprising up to 12 carbon atoms is a C₂-C₁₂ alkynyl, an alkynyl comprising up to 10 carbon atoms is a C₂-C₁₀ alkynyl, an alkynyl comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like.

“Aryl” refers to a hydrocarbon ring system radical comprising hydrogen, 6 to 18 carbon ring atoms and at least one aromatic ring. For purposes of this disclosure, the aryl radical can be a monocyclic, bicyclic, tricyclic or tetracyclic ring system, which can include fused, bridged, or spiro ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene.

“Cycloalkyl” refers to a stable non-aromatic monocyclic or polycyclic fully saturated hydrocarbon radical consisting solely of carbon and hydrogen atoms, which can include fused, bridged, or spiro ring systems, having from three to twenty carbon atoms, e.g., having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like.

“Heteroaryl” refers to a 5- to 20-membered ring system radical comprising one to thirteen carbon ring atoms, one to six heteroatoms as ring atoms selected from nitrogen, oxygen and sulfur, and at least one aromatic ring containing at least one heteroatom as a ring atom. For purposes of this disclosure, the heteroaryl radical can be a monocyclic, bicyclic, tricyclic or tetracyclic ring system, which can include fused, bridged, or spiro ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophene), benzotriazolyl, benzo[4,6]imidazo[1,2-a]pyridinyl, carbazolyl, cinnolinyl, dibenzofuranyl, dibenzothiophene, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophene (i.e. thienyl).

“Heterocyclyl” refers to a stable 3- to 20-membered non-aromatic, saturated or partially unsaturated ring radical which consists of two to twelve carbon ring atoms and from one to six heteroatoms as ring atoms selected from nitrogen, oxygen or sulfur, at least one non-aromatic, saturated or partially unsaturated ring containing at least one heteroatom as a ring atom. Unless stated otherwise specifically in the specification, the heterocyclyl radical can be a monocyclic, bicyclic, tricyclic or tetracyclic ring system, which can include fused, bridged, or spiro ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

As used herein, the symbol

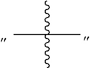

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

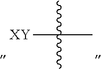

indicates that the chemical entity “XY” is bonded to another chemical entity via the point of attachment bond. Furthermore, the specific point of attachment to the non-depicted chemical entity can be specified by inference.

In this specification, unless stated otherwise, the term “pharmaceutically acceptable” is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

The term “pharmaceutically acceptable salt” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The compounds of the disclosure, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another.

### Compounds

The present disclosure provides compounds of formula (I), (I-A), (I-B) (I-C), (I-D), (I-E), Table A, or Table B or a pharmaceutically acceptable salt, a stereoisomer, or a deuterated form thereof.

In embodiments, the present disclosure provides a compound of formula (I):

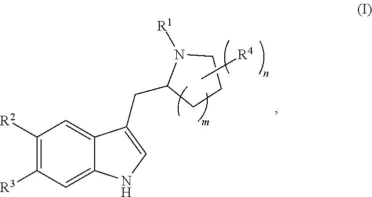

or a pharmaceutically acceptable salt, a stereoisomer, or a deuterated form thereof, wherein:
R¹ is —H, -alkyl, -alkenyl, -alkynyl, -aryl, -heteroaryl, -alkylene-aryl, -alkylene-heteroaryl, -heterocyclyl, -cycloalkyl, -alkylene-O-alkyl, or —O-alkyl; wherein the -alkyl, -alkenyl, -alkynyl, -aryl, -heteroaryl, -alkylene-aryl, -alkylene-heteroaryl, -heterocyclyl, -cycloalkyl, -alkylene-O-alkyl, or —O-alkyl are optionally substituted with one or more -halo, oxo, —OH, —NH₂, —CN, —O—C₁₋₆ alkyl, or —C₁₋₆ alkyl;
R² is -halo, —O-alkyl, —S-alkyl, —S(O)₂-alkyl, —SH, —NH— alkyl, —N(alkyl)₂, —NH₂, —CN, —O(C═O)-alkyl, or —NH(C═O)-alkyl;
R³ is -halo, —O-alkyl, —OH, —S-alkyl, —S(O)₂-alkyl, —SH, —NH— alkyl, —N(alkyl)₂, —NH₂, —CN, —O(C═O)-alkyl, or —NH(C═O)-alkyl; or
R² and R³ together with the carbon to which they are attached from a heterocyclyl;
R⁴ is -alkyl, -aryl, -heteroaryl, -alkylene-aryl, -alkylene-heteroaryl, -alkylene-OH, -alkylene-O-alkyl, or —O-alkyl;
n is 0, 1, 2, or 3; and
m is 0 or 1,
and wherein, the compound of Formula (I) is not:

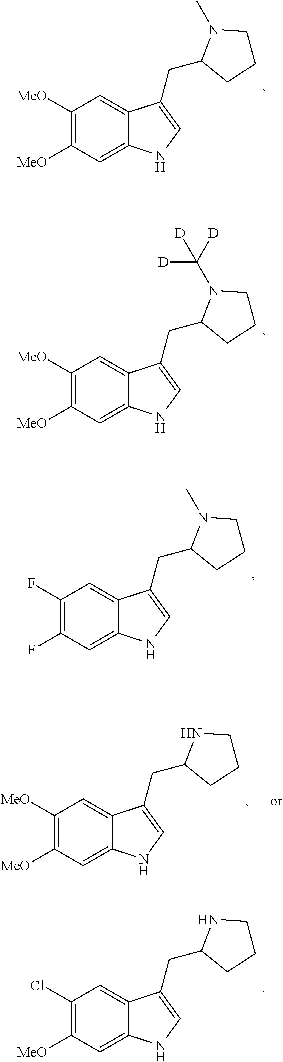 [SMILES: COc1cc2[nH]cc(CC3CCCN3C)c2cc1OC ; [2H]C([2H])([2H])N1CCCC1Cc1c[nH]c2cc(OC)c(OC)cc12 ; CN1CCCC1Cc1c[nH]c2cc(F)c(F)cc12 ; COc1cc2[nH]cc(CC3CCCN3)c2cc1OC ; COc1cc2[nH]cc(CC3CCCN3)c2cc1Cl]

In embodiments, the present disclosure provides a compound of formula (I):

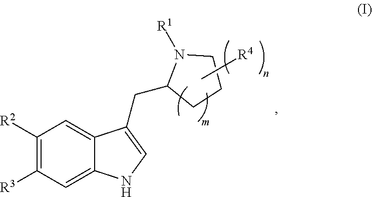

or a pharmaceutically acceptable salt, a stereoisomer, or a deuterated form thereof, wherein:
R¹ is —H, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl; wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more -halo, oxo, —OH, —NH₂, —CN, —O—C₁₋₆ alkyl, or —C₁₋₆ alkyl;
R² is -halo, —O—C₁₋₆ alkyl, —S—C₁₋₆ alkyl, —S(O)₂—C₁₋₆ alkyl, —SH, —NH—C₁₋₆ alkyl, —N(C₁₋₆ alkyl)₂, —NH₂, —CN, —O(C═O)—C₁₋₆ alkyl, or —NH(C═O)—C₁₋₆ alkyl;
R³ is -halo, —O—C₁₋₆ alkyl, —OH, —S—C₁₋₆ alkyl, —S(O)₂—C₁₋₆ alkyl, —SH, —NH—C₁₋₆ alkyl, —N(C₁₋₆ alkyl)₂, —NH₂, —CN, —O(C═O)—C₁₋₆ alkyl, or —NH(C═O)—C₁₋₆ alkyl; or
R² and R³ together with the carbon to which they are attached from a heterocyclyl;
R⁴ is —C₁₋₆ alkyl, -aryl, -heteroaryl, —C₁₋₆ alkylene-aryl, —C₁₋₆ alkylene-heteroaryl, —C₁₋₆ alkylene-OH, —C₁₋₆ alkylene-O—C₁₋₆ alkyl, or —O—C₁₋₆ alkyl;
n is 0, 1, 2, or 3; and
m is 0 or 1,
and wherein, the compound of Formula (I) is not:

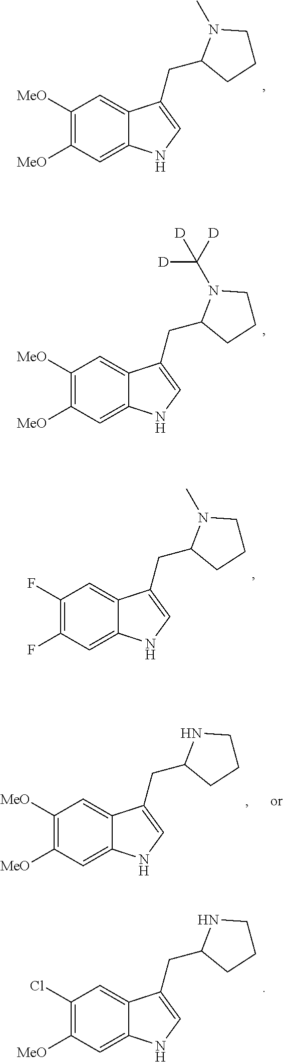 [SMILES: COc1cc2[nH]cc(CC3CCCN3C)c2cc1OC ; [2H]C([2H])([2H])N1CCCC1Cc1c[nH]c2cc(OC)c(OC)cc12 ; CN1CCCC1Cc1c[nH]c2cc(F)c(F)cc12 ; COc1cc2[nH]cc(CC3CCCN3)c2cc1OC ; COc1cc2[nH]cc(CC3CCCN3)c2cc1Cl]

In embodiments, the present disclosure provides a compound of formula (I-A):

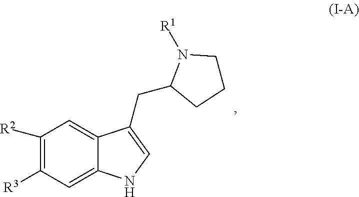

or a pharmaceutically acceptable salt, a stereoisomer, or a deuterated form thereof,
wherein: R¹, R³, and R⁴ are as described herein.

In embodiments, the present disclosure provides a compound of formula (I-B):

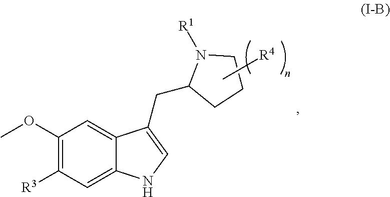

or a pharmaceutically acceptable salt, a stereoisomer, or a deuterated form thereof,
wherein: R¹, R³, and R⁴ are as described herein.

In embodiments, the present disclosure provides a compound of formula (I-C):

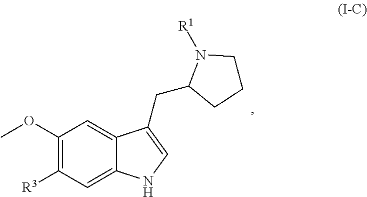

or a pharmaceutically acceptable salt, a stereoisomer, or a deuterated form thereof,
wherein: R¹ and R³ are as described herein.

In embodiments, the present disclosure provides a compound of formula (I-D):

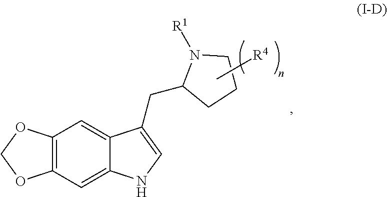

or a pharmaceutically acceptable salt, a stereoisomer, or a deuterated form thereof,
wherein: R¹ and R⁴ are as described herein.

In embodiments, the present disclosure provides a compound of formula (I-E):

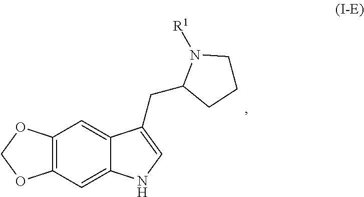

wherein: R¹ is as described herein.

### R¹

In embodiments of formula (I), (I-A), (I-B), (I-C), (I-D), or (I-E), R¹ is —H, -alkyl, -alkenyl, -alkynyl, -aryl, -heteroaryl, -alkylene-aryl, -alkylene-heteroaryl, -heterocyclyl, -cycloalkyl, -alkylene-O-alkyl, or —O-alkyl; wherein the -alkyl, -alkenyl, -alkynyl, -aryl, -heteroaryl, -alkylene-aryl, -alkylene-heteroaryl, -heterocyclyl, -cycloalkyl, -alkylene-O-alkyl, or —O-alkyl are optionally substituted with one or more -halo, oxo, —OH, —NH₂, —CN, —O—C₁₋₆ alkyl, or —C₁₋₆ alkyl. In embodiments of formula (I), (I-A), (I-B), (I-C), (I-D), or (I-E), R¹ is —H, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl; wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more -halo, oxo, —OH, —NH₂, —CN, —O—C₁₋₆ alkyl, or —C₁₋₆ alkyl.

In embodiments of formula (I), (I-A), (I-B), (I-C), (I-D), or (I-E), R¹ is —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl; wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more -halo, oxo, —OH, —NH₂, —CN, —O—C₁₋₆ alkyl, or —C₁₋₆ alkyl.

In embodiments of formula (I), (I-A), (I-B), (I-C), (I-D), or (I-E), R¹ is -alkyl, -alkenyl, or -alkynyl, each of which are optionally substituted with -halo, oxo, —OH, —NH₂, or —CN.

In embodiments of formula (I), (I-A), (I-B), (I-C), (I-D), or (I-E), R¹ is —C₁₋₆ alkyl, —C₂₋₆ alkenyl, or —C₂₋₆ alkynyl, each of which are optionally substituted with one or more -halo, oxo, —OH, —NH₂, or —CN. In embodiments of formula (I), (I-A), (I-B), (I-C), (I-D), or (I-E), R¹ is -substituted —C₁₋₆ alkyl or —C₁₋₆ alkylene-OH.

In embodiments of formula (I), (I-A), (I-B), (I-C), (I-D), or (I-E), R¹ is -aryl, -heteroaryl, -alkylene-aryl, -alkylene-heteroaryl, -heterocyclyl, or -cycloalkyl, wherein the -aryl, -heteroaryl, -alkylene-aryl, -alkylene-heteroaryl, -heterocyclyl, or -cycloalkyl are optionally substituted with one or more -halo, oxo, —OH, —NH₂, —CN, —O—C₁₋₆ alkyl, or —C₁₋₆ alkyl.

In embodiments of formula (I), (I-A), (I-B), (I-C), (I-D), or (I-E), R¹ is -aryl, -heteroaryl, —C₁₋₆ alkylene-aryl, —C₁₋₆ alkylene-heteroaryl, -heterocyclyl, or -cycloalkyl, wherein the -aryl, -heteroaryl, —C₁₋₆ alkylene-aryl, —C₁₋₆ alkylene-heteroaryl, -heterocyclyl, or -cycloalkyl are optionally substituted with one or more -halo, oxo, —OH, —NH₂, —CN, —O—C₁₋₆ alkyl, or —C₁₋₆ alkyl.

In embodiments of formula (I), (I-A), (I-B), (I-C), (I-D), or (I-E), R¹ is -alkylene-O-alkyl or —O-alkyl, wherein the -alkylene-O-alkyl or —O-alkyl are optionally substituted with one or more -halo, oxo, —OH, —NH₂, —CN, —O—C₁₋₆ alkyl, or —C₁₋₆ alkyl.

In embodiments of formula (I), (I-A), (I-B), (I-C), (I-D), or (I-E), R¹ is —C₁₋₆ alkylene-O—C₁₋₆ alkyl, or —O—C₁₋₆ alkyl, wherein the —C₁₋₆ alkylene-O—C₁₋₆ alkyl, or —O—C₁₋₆ alkyl are optionally substituted with one or more -halo, oxo, —OH, —NH₂, —CN, —O—C₁₋₆ alkyl, or —C₁₋₆ alkyl.

In embodiments of formula (I), (I-A), (I-B), (I-C), (I-D), or (I-E), R¹ is —H, -alkyl, -alkylene-aryl, alkylene-heteroaryl, heterocyclyl, cycloalkyl, or alkylene-OH.

In embodiments of formula (I), (I-A), (I-B), (I-C), (I-D), or (I-E), R¹ is —H, —C₁₋₆ alkyl, —C₁₋₆ alkylene-aryl, C₁₋₆ alkylene-heteroaryl, heterocyclyl, cycloalkyl, or —C₁₋₆ alkylene-OH. In embodiments of formula (I), (I-A), (I-B), (I-C), (I-D), or (I-E), R¹ is —C₁₋₆ alkyl, —C₁₋₆ alkylene-aryl, C₁₋₆ alkylene-heteroaryl, heterocyclyl, cycloalkyl, or —C₁₋₆ alkylene-OH.

In embodiments of formula (I), (I-A), (I-B), (I-C), (I-D), or (I-E), R¹ is —H.

In embodiments of formula (I), (I-A), (I-B), (I-C), (I-D), or (I-E), R¹ is -alkyl. In embodiments, R¹ is —C₁₋₆ alkyl. In embodiments, R¹ is methyl. In embodiments, R¹ is ethyl. In embodiments, R¹ is propyl. In embodiments, R¹ is butyl. In embodiments, R¹ is isopropyl.

In embodiments (I), (I-A), (I-B), (I-C), (I-D), or (I-E), R¹ is -alkylene-OH. In embodiments, R¹ is C₁₋₆ alkylene-OH. In embodiments, R¹ is —CH₂—OH. In embodiments, R¹ is —CH₂CH₂—OH. In embodiments, R¹ is —CH₂CH₂CH₂—OH.

In embodiments (I), (I-A), (I-B), (I-C), (I-D), or (I-E), R¹ is a substituted -alkyl. In embodiments, R¹ is a substituted —C₁₋₆ alkyl. In embodiments, R¹ is —CH₂C(CH₃)₂OH.

In embodiments of formula (I), (I-A), (I-B), (I-C), (I-D), or (I-E), R¹ is -alkylene-heteroaryl.

In embodiments of formula (I), (I-A), (I-B), (I-C), (I-D), or (I-E), R¹ is —C₁₋₆ alkylene-heteroaryl.

In embodiments of formula (I), (I-A), (I-B), (I-C), (I-D), or (I-E), R¹ is —C₁₋₆ alkylene-heteroaryl, wherein the heteroaryl is a 5-6 member ring with 1, 2, or 3 heteroatoms independently selected from —O or —N.

In embodiments of formula (I), (I-A), (I-B), (I-C), (I-D), or (I-E), R¹ is

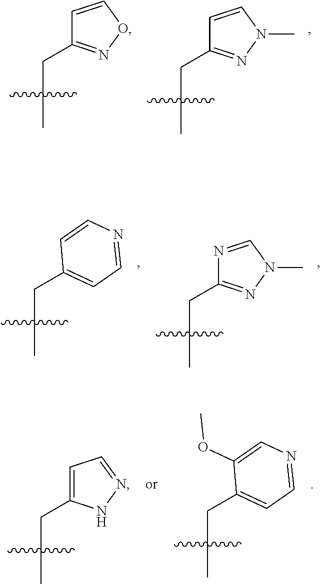

In embodiments of formula (I), (I-A), (I-B), (I-C), (I-D), or (I-E), R¹ is an alkylene-aryl.

In embodiments of formula (I), (I-A), (I-B), (I-C), (I-D), or (I-E), R¹ is

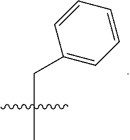 [SMILES: *Cc1ccccc1]

In embodiments of formula (I), (I-A), (I-B), (I-C), (I-D), or (I-E), R¹ is a heterocyclyl.

In embodiments of formula (I), (I-A), (I-B), (I-C), (I-D), or (I-E), R¹ is or

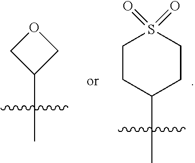 [SMILES: *C1COC1 ; *C1CCS(=O)(=O)CC1]

### R²

In embodiments, of formula (I) or (I-A), R² is -halo, —O-alkyl, —S-alkyl, —S(O)₂-alkyl, —SH, —NH— alkyl, —N(alkyl)₂, —NH₂, —CN, —O(C═O)-alkyl, or —NH(C═O)-alkyl.

In embodiments of formula (I) or (I-A), R² is -halo. In embodiments, R² is —F, —Cl, —Br, or —I. In embodiments, R² is —Cl, —Br, or —I. In embodiments, R² is —F, —Br, or —I. In embodiments, R² is —Br or —I.

In embodiments of formula (I) or (I-A), R²—Br, —I, —O—C₂₋₆ alkyl, —S-alkyl, —S(O)₂-alkyl, —SH, —NH-alkyl, —N(alkyl)₂, —NH₂, —CN, —O(C═O)-alkyl, or —NH(C═O)-alkyl.

In embodiments of formula (I) or (I-A), R²—Br, —I, —O—C₂₋₆ alkyl, —S—C₁₋₆ alkyl, —S(O)₂—C₁₋₆ alkyl, —SH, —NH—C₁₋₆ alkyl, —N(C₁₋₆ alkyl)₂, —NH₂, —CN, —O(C═O)—C₁₋₆ alkyl, or —NH(C═O)—C₁₋₆ alkyl.

In embodiments of formula (I) or (I-A), R² is —O-alkyl.

In embodiments of formula (I) or (I-A), R² is —O—C₁₋₆ alkyl.

In embodiments of formula (I) or (I-A), R² is —O-methyl.

In embodiments of formula (I) or (I-A), R² is —S-alkyl.

In embodiments of formula (I) or (I-A), R² is —S—C₁₋₆ alkyl.

In embodiments of formula (I) or (I-A), R² is —S-methyl.

In embodiments of formula (I) or (I-A), R² is —S-alkyl, —S(O)₂-alkyl, or —SH.

In embodiments of formula (I) or (I-A), R² is —S—C₁₋₆ alkyl, —S(O)₂—C₁₋₆ alkyl, or —SH.

In embodiments of formula (I) or (I-A), R² is —NH—C_alkyl, —N(alkyl)₂, —NH₂, or —CN.

In embodiments of formula (I) or (I-A), R² is —NH—C₁₋₆ alkyl, —N(C₁₋₆ alkyl)₂, —NH₂, or —CN.

In embodiments of formula (I) or (I-A), R² is —O(C═O)-alkyl or —NH(C═O)-alkyl.

In embodiments of formula (I) or (I-A), R² is —O(C═O)—C₁₋₆ alkyl or —NH(C═O)—C₁₋₆ alkyl.

In embodiments of formula (I) or (I-A), R² is —O-alkyl. In embodiments, R² is —O—C₁₋₆ alkyl. In embodiments of formula (I) or (I-A), R² is —O—C₂₋₆ alkyl.

In embodiments of formula (I) or (I-A), R² is —O-alkyl. In embodiments, R² is —O—C₁₋₆ alkyl. In embodiments, R² is —O—C₂₋₆ alkyl. In embodiments, R² is —O-methyl. In embodiments, R² is —O— ethyl. In embodiments, R² is —O-propyl. In embodiments, R² is —O-butyl.

In embodiments of formula (I), (I-A), (I-B), (I-C), R³ is -halo, —O-alkyl, —OH, —S-alkyl, —S(O)₂-alkyl, —SH, —NH— alkyl, —N(alkyl)₂, —NH₂, —CN, —O(C═O)-alkyl, or —NH(C═O)-alkyl.

In embodiments of formula (I), (I-A), (I-B), (I-C), R³ is —S-alkyl, —S(O)₂-alkyl, or —SH.

In embodiments of formula (I), (I-A), (I-B), (I-C), R³ is —S—C₁₋₆ alkyl, —S(O)₂—C₁₋₆ alkyl, or —SH.

In embodiments of formula (I), (I-A), (I-B), (I-C), R³ is —NH-alkyl, —N(alkyl)₂, —NH₂, or —CN.

In embodiments of formula (I), (I-A), (I-B), (I-C), R³ is —NH—C₁₋₆ alkyl, —N(C₁₋₆ alkyl)₂, —NH₂, or —CN.

In embodiments of formula (I), (I-A), (I-B), (I-C), R³ is —Cl, —Br, —I, —O—C₂₋₆ alkyl, —OH, —S-alkyl, —S(O)₂-alkyl, —SH, —NH-alkyl, —N(alkyl)₂, —NH₂, —CN, —O(C═O)-alkyl, or —NH(C═O)-alkyl.

In embodiments of formula (I), (I-A), (I-B), (I-C), R³ is —Cl, —Br, —I, —O—C₂₋₆ alkyl, —OH, —S—C₁₋₆ alkyl, —S(O)₂—C₁₋₆ alkyl, —SH, —NH—C₁₋₆ alkyl, —N(C₁₋₆ alkyl)₂, —NH₂, —CN, —O(C═O)—C₁₋₆ alkyl, or —NH(C═O)—C₁₋₆ alkyl.

In embodiments of formula (I), (I-A), (I-B), (I-C), R³ is —O(C═O)-alkyl or —NH(C═O)— alkyl.

In embodiments of formula (I), (I-A), (I-B), (I-C), R³ is —O(C═O)—C₁₋₆ alkyl or —NH(C═O)—C₁₋₆ alkyl.

In embodiments of formula (I), (I-A), (I-B), or (I-C), R³ is -halo or -alkyl.

In embodiments of formula (I), (I-A), (I-B), or (I-C), R³ is -halo or —C₁₋₆ alkyl.

In embodiments of formula (I), (I-A), (I-B), (I-C), R³ is -halo. In embodiments of formula (I), (I-A), (I-B), or (I-C), R³ is —F, —Cl, —Br, or —I. In embodiments, R³ is —Cl, —Br, or —I. In embodiments, R³ is —F, —Br, or —I. In embodiments, R³ is —Br or —I.

In embodiments, R³ is —F. In embodiments, R³ is —Cl. In embodiments, R³ is —Br. In embodiments, R³ is —I.

In embodiments of formula (I), (I-A), (I-B), or (I-C), R³ is -alkyl. In embodiments, R³ is —C₁₋₆ alkyl. In embodiments, R³ is methyl. In embodiments, R³ is ethyl. In embodiments, R³ is propyl. In embodiments, R³ is butyl. In embodiments, R¹ is isopropyl.

R² and R³

In embodiments of formula (I) and (I-A), R² and R³ are not the same.

In embodiments of formula (I) and (I-A), R² is not —Cl and R³ is not —O-methyl.

In embodiments of formula (I) and (I-A), R² and R³ are not —F.

In embodiments of formula (I) and (I-A), R² and R³ are not —O-methyl.

In embodiments of formula (I) and (I-A), R² is —O-alkyl or —S-alkyl and R³ is halo.

In embodiments of formula (I) and (I-A), R² is —O-alkyl or —S-alkyl and R³ is —F.

In embodiments of formula (I) and (I-A), R² is —O—C₁₋₆ alkyl or —S—C₁₋₆ alkyl and R³ is halo.

In embodiments of formula (I) and (I-A), R² is —O—C₁₋₆ alkyl or —S—C₁₋₆ alkyl and R³ is —F.

In embodiments of formula (I) and (I-A), R² is —O-methyl or —S-methyl and R³ is —F.

In embodiments of formula (I) and (I-A), R² is —O-methyl and R³ is —F.

In embodiments of formula (I) and (I-A), R² is —S-methyl and R³ is —F.

In embodiments of formula (I) and (I-A), R² and R³ are each independently —O—C₂₋₆ alkyl, —S-alkyl, —S(O)₂-alkyl, —SH, —NH-alkyl, —N(alkyl)₂, —NH₂, —CN, —O(C═O)-alkyl, or —NH(C═O)— alkyl.

In embodiments of formula (I) and (I-A), R² and R³ are each independently —Br, —I, —O—C₂₋₆ alkyl, —S-alkyl, —S(O)₂-alkyl, —SH, —NH-alkyl, —N(alkyl)₂, —NH₂, —CN, —O(C═O)-alkyl, or —NH(C═O)-alkyl.

In embodiments of formula (I) and (I-A), R² and R³ are each independently —O—C₂₋₆ alkyl, —S—C₁₋₆ alkyl, —S(O)₂—C₁₋₆ alkyl, —SH, —NH—C₁₋₆ alkyl, —N(C₁₋₆ alkyl)₂, —NH₂, —CN, —O(C═O)—C₁₋₆ alkyl, or —NH(C═O)—C₁₋₆ alkyl.

In embodiments of formula (I) and (I-A), R² and R³ are each independently —Br, —I, —O—C₂₋₆ alkyl, —S—C₁₋₆ alkyl, —S(O)₂—C₁₋₆ alkyl, —SH, —NH—C₁₋₆ alkyl, —N(C₁₋₆ alkyl)₂, —NH₂, —CN, —O(C═O)—C₁₋₆ alkyl, or —NH(C═O)—C₁₋₆ alkyl.

In embodiments of formula (I) and (I-A), R² and R³ together with the carbon to which they are attached from a heterocyclyl.

In embodiments, the heterocyclyl is

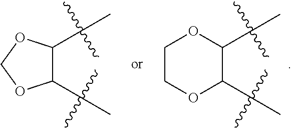 [SMILES: *C1OCOC1* ; *C1OCCOC1*]

In embodiments, the heterocyclyl is

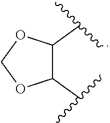 [SMILES: *C1OCOC1*]

In embodiments, the heterocyclyl is

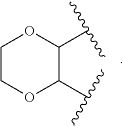 [SMILES: *C1OCCOC1*]

### R⁴

In embodiments of formula (I), (I-B), or (I-D), R⁴ is -alkyl, -aryl, -heteroaryl, -alkylene-aryl, -alkylene-heteroaryl, -alkylene-OH, -alkylene-O-alkyl, or —O-alkyl.

In embodiments of formula (I), (I-B), or (I-D), R⁴ is -alkyl, aryl, or -heteroaryl.

In embodiments of formula (I), (I-B), or (I-D), R⁴ is —C₁₋₆ alkyl, aryl, or -heteroaryl.

In embodiments of formula (I), (I-B), or (I-D), R⁴ is aryl or -heteroaryl.

In embodiments of formula (I), (I-B), or (I-D), R⁴ is -alkylene-aryl, -alkylene-heteroaryl, -alkylene-OH, or -alkylene-O-alkyl.

In embodiments of formula (I), (I-B), or (I-D), R⁴ is —C₁₋₆ alkylene-aryl, —C₁₋₆ alkylene-heteroaryl, —C₁₋₆ alkylene-OH, or —C₁₋₆ alkylene-O—C₁₋₆ alkyl.

In embodiments of formula (I), (I-B), or (I-D), R⁴ is -alkylene-aryl or -alkylene-heteroaryl.

In embodiments of formula (I), (I-B), or (I-D), R⁴ is —C₁₋₆ alkylene-aryl or —C₁₋₆ alkylene-heteroaryl.

In embodiments of formula (I), (I-B), or (I-D), R⁴ is -alkylene-OH or -alkylene-O-alkyl.

In embodiments of formula (I), (I-B), or (I-D), R⁴ is —C₁₋₆ alkylene-OH or —C₁₋₆ alkylene-O—C₁₋₆ alkyl.

In embodiments of formula (I), (I-B), or (I-D), R⁴ is —O-alkyl. In embodiments, R⁴ is —O—C₁₋₆ alkyl. In embodiments, R² is —O-methyl. In embodiments, R² is —O-ethyl. In embodiments, R² is —O-propyl. In embodiments, R² is —O-butyl.

n

In embodiments of formula (I), (I-B), or (I-D), n is 0.

In embodiments of formula (I), (I-B), or (I-D), n is 1.

In embodiments of formula (I), (I-B), or (I-D), n is 2.

In embodiments of formula (I), (I-B), or (I-D), n is 3.

m

In embodiments of formula (I), m is 0.

In embodiments of formula (I), m is 1.

In embodiments, provided herein is a compound in Table A, or a pharmaceutically acceptable salt thereof, racemic form thereof, or stereoisomer thereof.

In embodiments, provided herein is a compound in Table A, or a pharmaceutically acceptable salt thereof, or stereoisomer thereof.

In embodiments, provided herein is a compound in Table A, or a pharmaceutically acceptable salt thereof.

In one embodiment, provided herein is a compound set forth in Table A.

In some embodiments, provided herein is a pharmaceutically acceptable salt of a compound in Table A.

TABLE A
Various compounds of the present disclosure

| Compound | |
|---|---|
| No. | Structure |
| 1 | 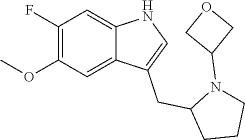 [SMILES: COc1cc2c(CC3CCCN3C3COC3)c[nH]c2cc1F] |
| 2 | 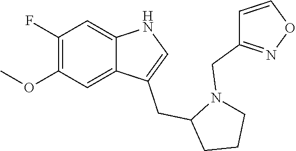 [SMILES: COc1cc2c(CC3CCCN3Cc3ccon3)c[nH]c2cc1F] |
| 3 | 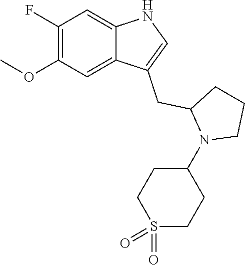 [SMILES: COc1cc2c(CC3CCCN3C3CCS(=O)(=O)CC3)c[nH]c2cc1F] |
| 4 | 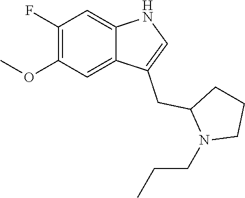 [SMILES: CCCN1CCCC1Cc1c[nH]c2cc(F)c(OC)cc12] |
| 5 | 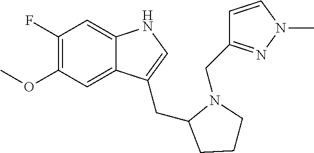 |
| 6 | 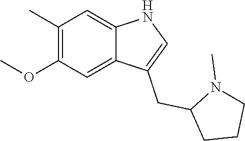 [SMILES: COc1cc2c(CC3CCCN3C)c[nH]c2cc1C] |
| 7 | 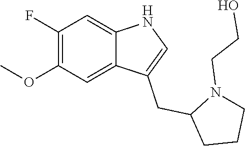 [SMILES: COc1cc2c(CC3CCCN3CCO)c[nH]c2cc1F] |
| 8 | 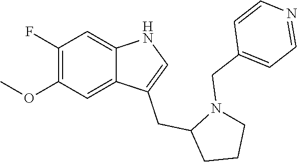 [SMILES: COc1cc2c(CC3CCCN3Cc3ccncc3)c[nH]c2cc1F] |
| 9 | 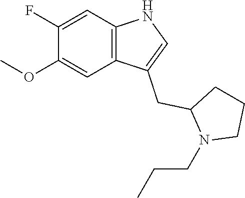 [SMILES: CCCN1CCCC1Cc1c[nH]c2cc(F)c(OC)cc12] |
| 10 | 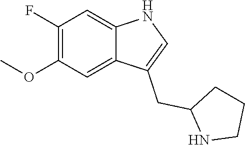 [SMILES: COc1cc2c(CC3CCCN3)c[nH]c2cc1F] |
| 11 | 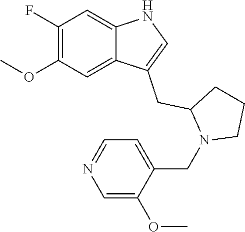 [SMILES: COc1cc2c(CC3CCCN3Cc3ccncc3OC)c[nH]c2cc1F] |
| 12 | 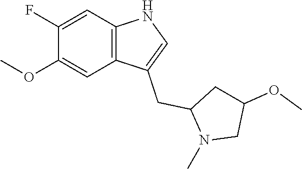 |
| 13 | 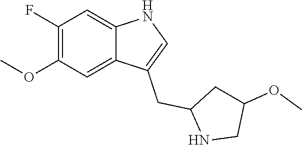 |
| 14 | 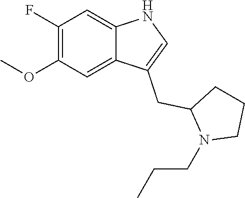 [SMILES: CCCN1CCCC1Cc1c[nH]c2cc(F)c(OC)cc12] |
| 15 | 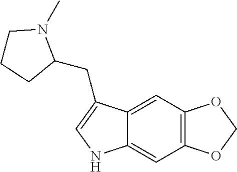 [SMILES: CN1CCCC1Cc1c[nH]c2cc3c(cc12)OCO3] |
| 16 | 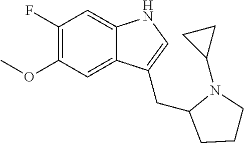 [SMILES: COc1cc2c(CC3CCCN3C3CC3)c[nH]c2cc1F] |
| 17 | 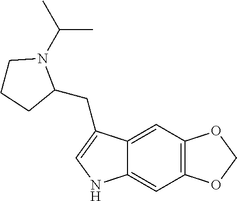 [SMILES: CC(C)N1CCCC1Cc1c[nH]c2cc3c(cc12)OCO3] |
| 18 | 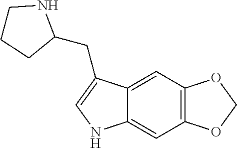 [SMILES: c1[nH]c2cc3c(cc2c1CC1CCCN1)OCO3] |
| 19 | 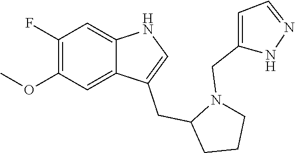 [SMILES: COc1cc2c(CC3CCCN3Cc3ccn[nH]3)c[nH]c2cc1F] |
| 20 | 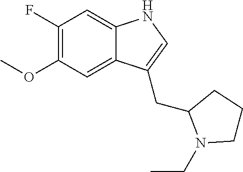 [SMILES: CCN1CCCC1Cc1c[nH]c2cc(F)c(OC)cc12] |
| 21 | 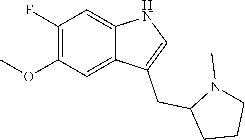 [SMILES: COc1cc2c(CC3CCCN3C)c[nH]c2cc1F] |
| 22 | 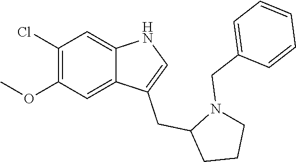 [SMILES: COc1cc2c(CC3CCCN3Cc3ccccc3)c[nH]c2cc1Cl] |
| 23 | 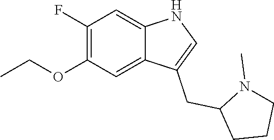 [SMILES: CCOc1cc2c(CC3CCCN3C)c[nH]c2cc1F] |
| 24 | 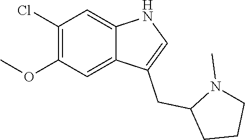 [SMILES: COc1cc2c(CC3CCCN3C)c[nH]c2cc1Cl] |
| 25 | 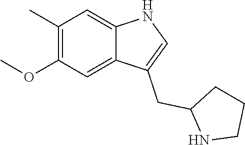 [SMILES: COc1cc2c(CC3CCCN3)c[nH]c2cc1C] |
| 26 | 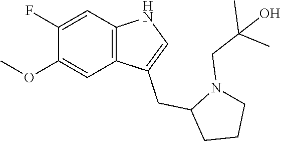 [SMILES: COc1cc2c(CC3CCCN3CC(C)(C)O)c[nH]c2cc1F] |
| 27 | 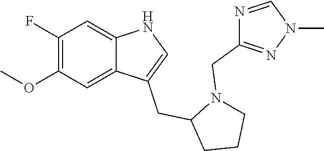 |
| 28 | 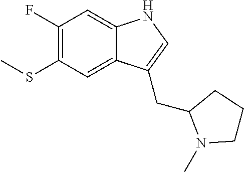 [SMILES: CSc1cc2c(CC3CCCN3C)c[nH]c2cc1F] |
| 29 | 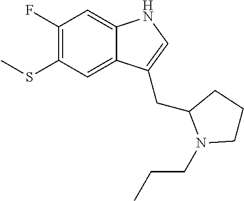 [SMILES: CCCN1CCCC1Cc1c[nH]c2cc(F)c(SC)cc12] |
| 30 | 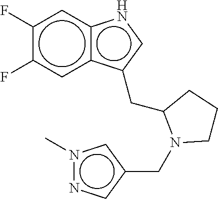 |
| 31 | 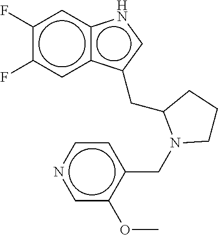 |

TABLE B
Various compounds of the present disclosure

| Compound | |
|---|---|
| No. | Structure |
| 1A | 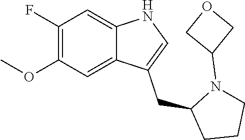 [SMILES: COc1cc2c(C[C@@H]3CCCN3C3COC3)c[nH]c2cc1F] |
| 1B | 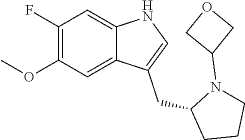 [SMILES: COc1cc2c(C[C@H]3CCCN3C3COC3)c[nH]c2cc1F] |
| 2A | 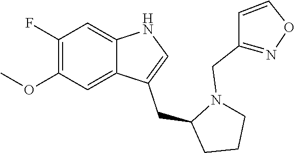 [SMILES: COc1cc2c(C[C@@H]3CCCN3Cc3ccon3)c[nH]c2cc1F] |
| 2B | 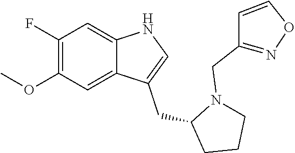 [SMILES: COc1cc2c(C[C@H]3CCCN3Cc3ccon3)c[nH]c2cc1F] |
| 3A | 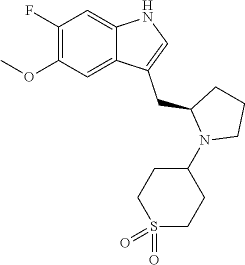 [SMILES: COc1cc2c(C[C@H]3CCCN3C3CCS(=O)(=O)CC3)c[nH]c2cc1F] |
| 3B | 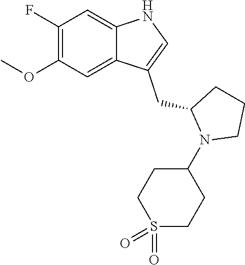 [SMILES: COc1cc2c(C[C@@H]3CCCN3C3CCS(=O)(=O)CC3)c[nH]c2cc1F] |
| 4A | 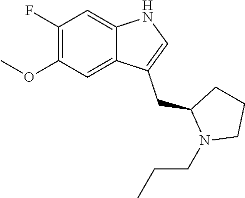 [SMILES: CCCN1CCC[C@@H]1Cc1c[nH]c2cc(F)c(OC)cc12] |
| 4B | 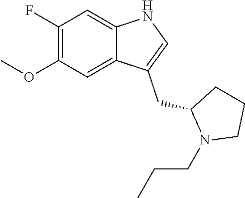 [SMILES: CCCN1CCC[C@H]1Cc1c[nH]c2cc(F)c(OC)cc12] |
| 5A | 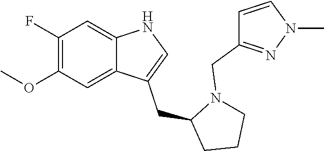 |
| 5B | 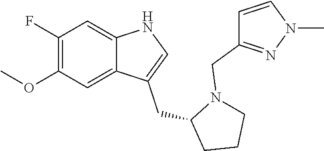 |
| 6A | 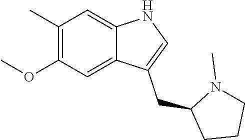 [SMILES: COc1cc2c(C[C@@H]3CCCN3C)c[nH]c2cc1C] |
| 6B | 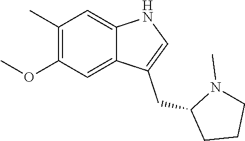 [SMILES: COc1cc2c(C[C@H]3CCCN3C)c[nH]c2cc1C] |
| 7A | 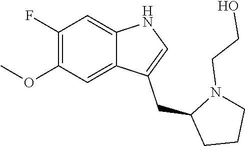 [SMILES: COc1cc2c(C[C@@H]3CCCN3CCO)c[nH]c2cc1F] |
| 7B | 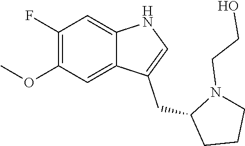 [SMILES: COc1cc2c(C[C@H]3CCCN3CCO)c[nH]c2cc1F] |
| 8A | 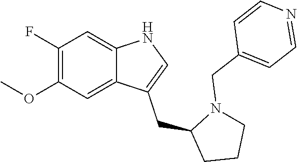 [SMILES: COc1cc2c(C[C@@H]3CCCN3Cc3ccncc3)c[nH]c2cc1F] |
| 8B | 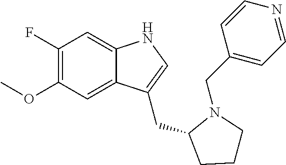 [SMILES: COc1cc2c(C[C@H]3CCCN3Cc3ccncc3)c[nH]c2cc1F] |
| 9A | 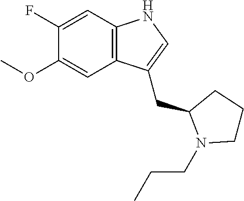 [SMILES: CCCN1CCC[C@@H]1Cc1c[nH]c2cc(F)c(OC)cc12] |
| 9B | 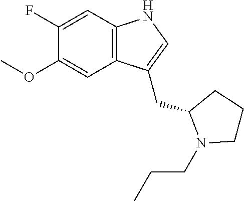 [SMILES: CCCN1CCC[C@H]1Cc1c[nH]c2cc(F)c(OC)cc12] |
| 10A | 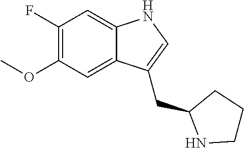 [SMILES: COc1cc2c(C[C@H]3CCCN3)c[nH]c2cc1F] |
| 10B | 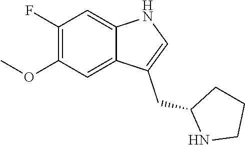 [SMILES: COc1cc2c(C[C@@H]3CCCN3)c[nH]c2cc1F] |
| 11A | 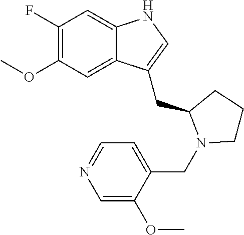 [SMILES: COc1cc2c(C[C@H]3CCCN3Cc3ccncc3OC)c[nH]c2cc1F] |
| 11B | 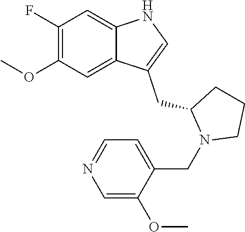 [SMILES: COc1cc2c(C[C@@H]3CCCN3Cc3ccncc3OC)c[nH]c2cc1F] |
| 12A | 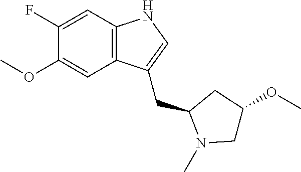 |
| 12B | 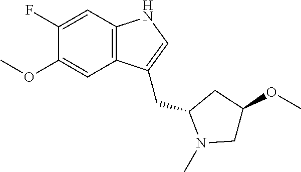 |
| 12C | 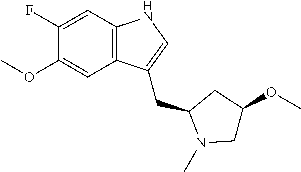 |
| 12D | 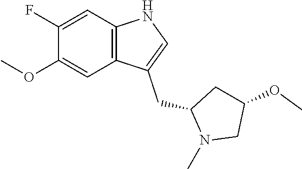 |
| 13A | 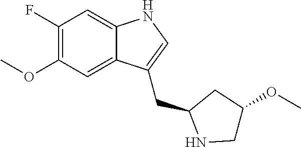 |
| 13B | 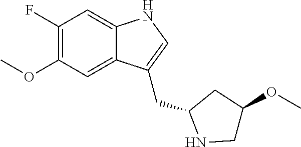 |
| 13C | 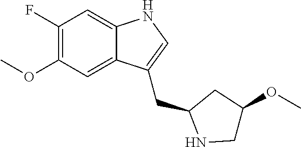 |
| 13D | 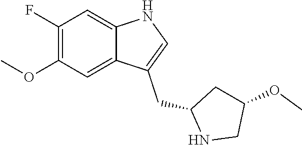 |
| 14A | 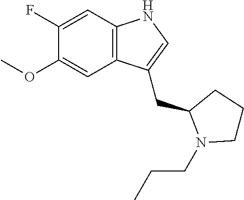 [SMILES: CCCN1CCC[C@@H]1Cc1c[nH]c2cc(F)c(OC)cc12] |
| 14B | 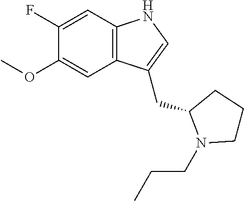 [SMILES: CCCN1CCC[C@H]1Cc1c[nH]c2cc(F)c(OC)cc12] |
| 15A | 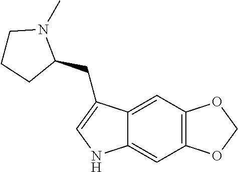 [SMILES: CN1CCC[C@@H]1Cc1c[nH]c2cc3c(cc12)OCO3] |
| 15B | 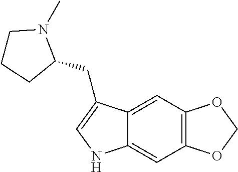 [SMILES: CN1CCC[C@H]1Cc1c[nH]c2cc3c(cc12)OCO3] |
| 16A | 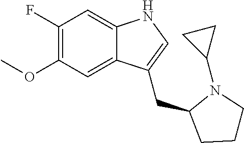 [SMILES: COc1cc2c(C[C@@H]3CCCN3C3CC3)c[nH]c2cc1F] |
| 16B | 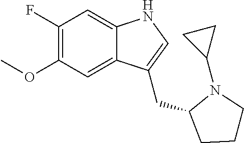 [SMILES: COc1cc2c(C[C@H]3CCCN3C3CC3)c[nH]c2cc1F] |
| 17A | 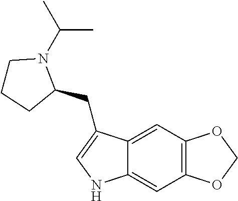 [SMILES: CC(C)N1CCC[C@@H]1Cc1c[nH]c2cc3c(cc12)OCO3] |
| 17B | 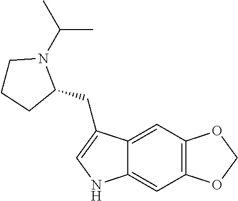 [SMILES: CC(C)N1CCC[C@H]1Cc1c[nH]c2cc3c(cc12)OCO3] |
| 18A | 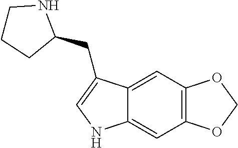 [SMILES: c1[nH]c2cc3c(cc2c1C[C@H]1CCCN1)OCO3] |
| 18B | 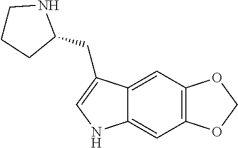 [SMILES: c1[nH]c2cc3c(cc2c1C[C@@H]1CCCN1)OCO3] |
| 19A | 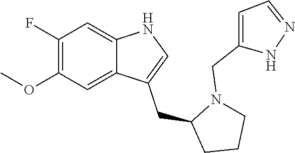 [SMILES: COc1cc2c(C[C@@H]3CCCN3Cc3ccn[nH]3)c[nH]c2cc1F] |
| 19B | 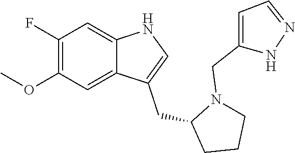 [SMILES: COc1cc2c(C[C@H]3CCCN3Cc3ccn[nH]3)c[nH]c2cc1F] |
| 20A | 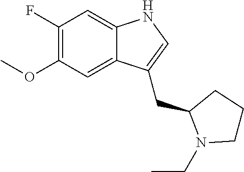 [SMILES: CCN1CCC[C@@H]1Cc1c[nH]c2cc(F)c(OC)cc12] |
| 20B | 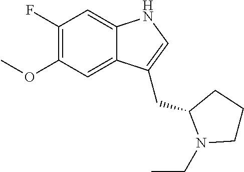 [SMILES: CCN1CCC[C@H]1Cc1c[nH]c2cc(F)c(OC)cc12] |
| 21A | 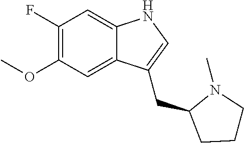 [SMILES: COc1cc2c(C[C@@H]3CCCN3C)c[nH]c2cc1F] |
| 21B | 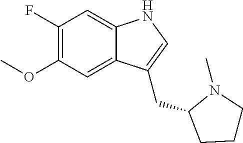 [SMILES: COc1cc2c(C[C@H]3CCCN3C)c[nH]c2cc1F] |
| 22A | 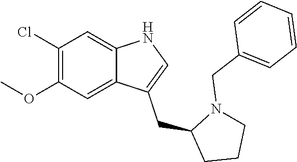 [SMILES: COc1cc2c(C[C@@H]3CCCN3Cc3ccccc3)c[nH]c2cc1Cl] |
| 22B | 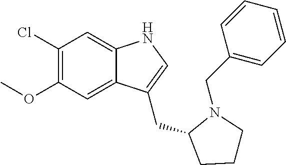 [SMILES: COc1cc2c(C[C@H]3CCCN3Cc3ccccc3)c[nH]c2cc1Cl] |
| 23A | 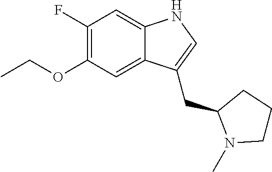 [SMILES: CCOc1cc2c(C[C@H]3CCCN3C)c[nH]c2cc1F] |
| 23B | 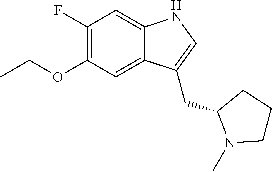 [SMILES: CCOc1cc2c(C[C@@H]3CCCN3C)c[nH]c2cc1F] |
| 24A | 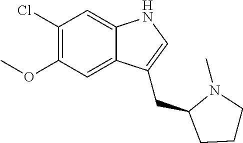 [SMILES: COc1cc2c(C[C@@H]3CCCN3C)c[nH]c2cc1Cl] |
| 24B | 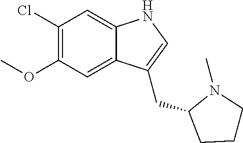 [SMILES: COc1cc2c(C[C@H]3CCCN3C)c[nH]c2cc1Cl] |
| 25A | 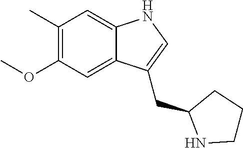 [SMILES: COc1cc2c(C[C@H]3CCCN3)c[nH]c2cc1C] |
| 25B | 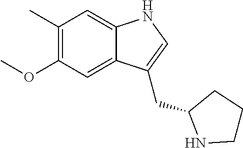 [SMILES: COc1cc2c(C[C@@H]3CCCN3)c[nH]c2cc1C] |
| 26A | 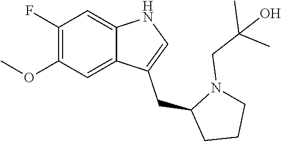 [SMILES: COc1cc2c(C[C@@H]3CCCN3CC(C)(C)O)c[nH]c2cc1F] |
| 26B | 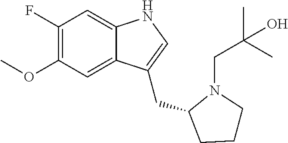 [SMILES: COc1cc2c(C[C@H]3CCCN3CC(C)(C)O)c[nH]c2cc1F] |
| 27A | 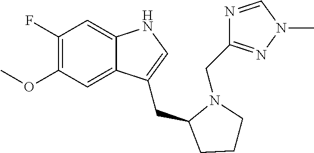 |
| 27B | 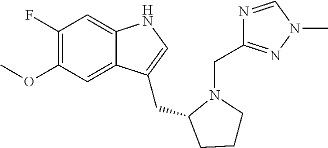 |
| 28A | 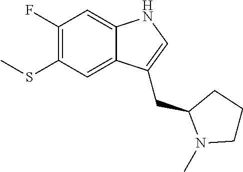 [SMILES: CSc1cc2c(C[C@H]3CCCN3C)c[nH]c2cc1F] |
| 28B | 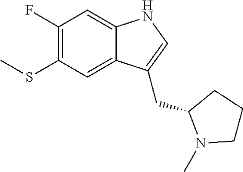 [SMILES: CSc1cc2c(C[C@@H]3CCCN3C)c[nH]c2cc1F] |
| 29A | 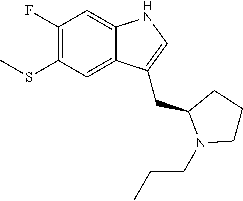 [SMILES: CCCN1CCC[C@@H]1Cc1c[nH]c2cc(F)c(SC)cc12] |
| 29B | 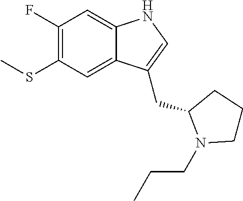 [SMILES: CCCN1CCC[C@H]1Cc1c[nH]c2cc(F)c(SC)cc12] |
| 30A | 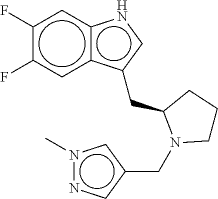 |
| 30B | 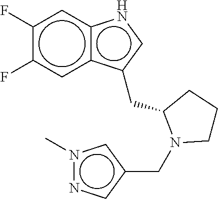 |
| 31A | 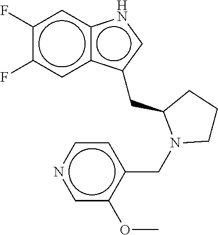 |
| 31B | 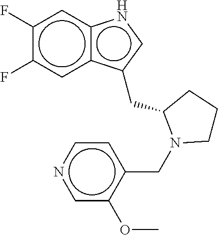 |

### Compositions

The present disclosure provides pharmaceutical compositions comprising at least one compound disclosed herein (e.g., of compound of formula (I), (I-A), (I-B), (I-C), (I-D), or (I-E), Table A or Table B or pharmaceutically acceptable salt, deuterated versions, or stereoisomer thereof) and one or more pharmaceutically acceptable excipients.

The compounds provided herein (e.g., of compound of formula (I), (I-A), (I-B), (I-C), (I-D), or (I-E), Table A or Table B or pharmaceutically acceptable salt, deuterated versions, or stereoisomer thereof) may be administered as compounds per se or may be formulated as pharmaceutical compositions. The pharmaceutical compositions may comprise one or more pharmaceutically acceptable excipients, such as carriers, diluents, fillers, disintegrants, lubricating agents, binders, colorants, pigments, stabilizers, preservatives, and/or antioxidants.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

### Therapeutic Use

The disclosure further relates to compounds disclosed herein (e.g., of compound of formula (I), (I-A), (I-B), (I-C), (I-D), or (I-E), Table A or Table B or pharmaceutically acceptable salt, deuterated versions, or stereoisomer thereof), or a pharmaceutical composition comprising at least one compound disclosed herein (e.g., of compound of formula (I), (I-A), (I-B), (I-C), (I-D), or (I-E), Table A or Table B or pharmaceutically acceptable salt, deuterated versions, or stereoisomer thereof), for use in the treatment of a serotonin 5-HT2A receptor associated disease/disorder.

In embodiments, the compounds (e.g., of compound of formula (I), (I-A), (I-B), (I-C), (I-D), or (I-E), Table A or Table B or pharmaceutically acceptable salt, deuterated versions, or stereoisomer thereof) may be used in the treatment of an anxiety disorder, attention deficit hyperactivity disorder (ADHD), depression (including treatment resistant depression), cluster headache, diminished drive, burn-out, bore-out, migraine, Parkinson's disease, schizophrenia, an eating disorder (including anorexia nervosa), psychotic disorder, schizophrenia, schizophreniform disorder, schizoaffective disorder, bipolar I disorder, bipolar II disorder, major depressive disorder, psychotic depression, delusional disorders, shared psychotic disorder, Shared paranoia disorder, brief psychotic disorder, paranoid personality disorder, schizoid personality disorder, schizotypal personality disorder, social anxiety disorder, substance-induced anxiety disorder, selective mutism, panic disorder, panic attacks, agoraphobia, posttraumatic stress disorder (PTSD), premenstrual dysphoric disorder (PMDD), and premenstrual syndrome (PMS).

In embodiments, the 5-HT₂A receptor associated disease or disorder is depression. In embodiments, the depression is treatment resistant depression.

In embodiments, the 5-HT₂A receptor associated disease or disorder is an eating disorder. In embodiments, the eating disorder is anorexia nervosa.

In embodiments, the 5-HT₂A receptor associated disease or disorder is an anxiety disorder.

In embodiments, the 5-HT₂A receptor associated disease or disorder is bipolar I disorder.

In embodiments, the 5-HT₂A receptor associated disease or disorder is bipolar II disorder.

In embodiments, the 5-HT₂A receptor associated disease or disorder is major depressive disorder.

In embodiments, the 5-HT₂A receptor associated disease or disorder is posttraumatic stress disorder (PTSD).

### EXAMPLES

The present disclosure is further illustrated by reference to the following Examples. However, it should be noted that these Examples, like the embodiments described above, are illustrative and are not to be construed as restricting the scope of the disclosure in any way.

In embodiments, compounds of the present disclosure can be synthesized using the following methods. General reaction conditions are given, and reaction products can be purified by generally known methods including silica gel chromatography using various organic solvents such as hexane, dichloromethane, ethyl acetate, methanol and the like or preparative reverse phase high pressure liquid chromatography.

### Example 1: Synthesis of (R)-6-Fluoro-5-methoxy-3-((1-methylpyrrolidin-2-yl)methyl)-1H-indole (Compound 21B)

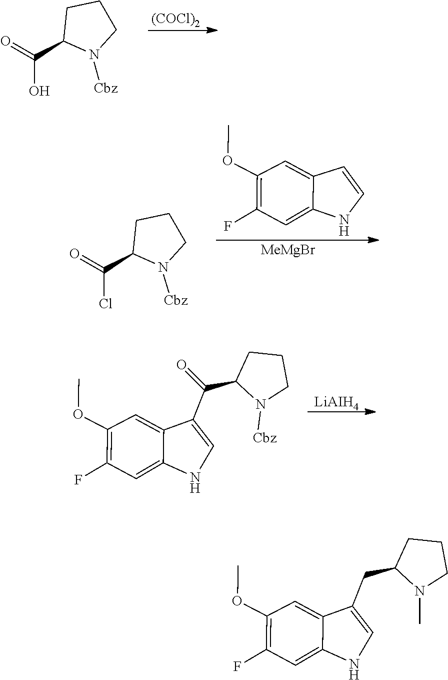

### Step 1: Benzyl (R)-2-(chlorocarbonyl)pyrrolidine-1-carboxylate

To a stirred solution of ((benzyloxy)carbonyl)-D-proline (4.00 g, 1 Eq, 16.0 mmol) in dry DCM (40 mL) under a nitrogen atmosphere at 0° C. was added oxalyl dichloride (7.25 g, 5.0 mL, 3.6 Eq, 57.1 mmol). The reaction mixture was stirred at 22° C. for 3.5 h. The reaction mixture was concentrated in vacuo, and the resulting crude was suspended in isohexane (3×20 mL) and dried in vacuo to provide the sub-title compound (4.30 g, 15 mmol, 94%) as a yellow oil.

m/z 220.6 (M−CO₂Cl+MeOH)⁺ (ES+)

### Step 2: Benzyl (R)-2-(6-fluoro-5-methoxy-1H-indole-3-carbonyl)pyrrolidine-1-carboxylate

To a stirred solution of 6-fluoro-5-methoxy-1H-indole (1.05 g, 1.8 Eq, 6.36 mmol) in dry toluene (10.0 mL) under a nitrogen atmosphere at 22° C. was added MeMgBr in Et₂O (0.82 g, 2.3 mL, 3.00 molar, 1.9 Eq, 6.9 mmol) dropwise over 2 minutes. THF (4 mL) was added. The reaction mixture was stirred at rt for 4 h. the product from step 1 above (0.97 g, 1 Eq, 3.6 mmol) in dry toluene (10.0 mL) was added dropwise and the reaction mixture was stirred at rt for 18 h. The reaction mixture was quenched with sat. aq. NaHCO₃ (500 mL) and left to stir at rt for 1 h. The resulting suspension was extracted with EtOAc (3×50 mL), and the collected organic layers were dried (Na₂SO₄) and concentrated in vacuo. The crude product was purified by chromatography on silica gel (40 g cartridge, 25-100% EtOAc/isohexane) to afford the sub-title compound (766 mg, 1.6 mmol, 45%).

m/z 397.1 (M+H)⁺ (ES+); 395.3 (M−H)⁻ (ES⁻)

¹H NMR (500 MHz, DMSO) δ 11.95 (s, 1H), 8.39 (d, J=10.7 Hz, 1H), 7.85 (dd, J=15.3, 8.7 Hz, 1H), 7.40-7.36 (m, 2H), 7.36-7.31 (m, 1H), 7.17-6.99 (m, 3H), 5.25-5.15 (m, 1H), 5.09-5.01 (m, 1H), 4.94 (q, J=13.2 Hz, 1H), 3.86 (d, J=3.7 Hz, 3H), 3.56-3.45 (m, 2H), 2.46-2.27 (m, 1H), 1.91-1.80 (m, 3H).

¹⁹F NMR (471 MHz, DMSO) δ −138.29-−138.38 (m).

### Step 3: (R)-6-Fluoro-5-methoxy-3-((1-methylpyrrolidin-2-yl)methyl)-1H-indole

To a stirred solution of the product from step 2 above (100 mg, 85% Wt, 1 Eq, 214 μmol) in 2-Me-THF (5.00 mL) at 0° C. was added LiAlH₄ in THF (0.36 g, 400 μL, 2.40 molar, 4.48 Eq, 960 μmol). The reaction mixture was heated to 80° C. and stirred for 2 h. The reaction mixture was cooled to 0° C. and quenched by slow addition of THF/H₂O (4:1, 5 mL). To the mixture was added Na₂SO₄ followed by stirring at rt for 30 min before it was filtered, washing with THF (5 mL). The filtrate was concentrated in vacuo. The crude product was purified by chromatography on silica gel (4 g cartridge, 0-100% (2% NH₄OH in 3:1 EtOAc/EtOH) in Isohexane) to afford the title compound (50.0 mg, 0.18 mmol, 84%).

m/z 263.5 (M+H)⁺ (ES+); 261.1 (M−H)⁻ (ES⁻)

¹H NMR (500 MHz, MeOD) δ 7.12 (d, J=8.3 Hz, 1H), 7.08-7.04 (m, 1H), 7.01 (s, 1H), 3.88 (s, 3H), 3.17-3.08 (m, 2H), 2.66-2.51 (m, 2H), 2.45 (s, 3H), 2.34-2.25 (m, 1H), 1.92-1.81 (m, 1H), 1.81-1.65 (m, 3H), 1.65-1.53 (m, 1H).

¹⁹F NMR (471 MHz, MeOD) δ −143.30.

ee of 41%.

### Example 2: Synthesis of (R)-6-Fluoro-5-methoxy-3-(pyrrolidin-2-ylmethyl)-1H-indole, Formic Acid and (R)-6-Fluoro-5-methoxy-3-((1-propylpyrrolidin-2-yl)methyl)-1H-indole (Compound 14A)

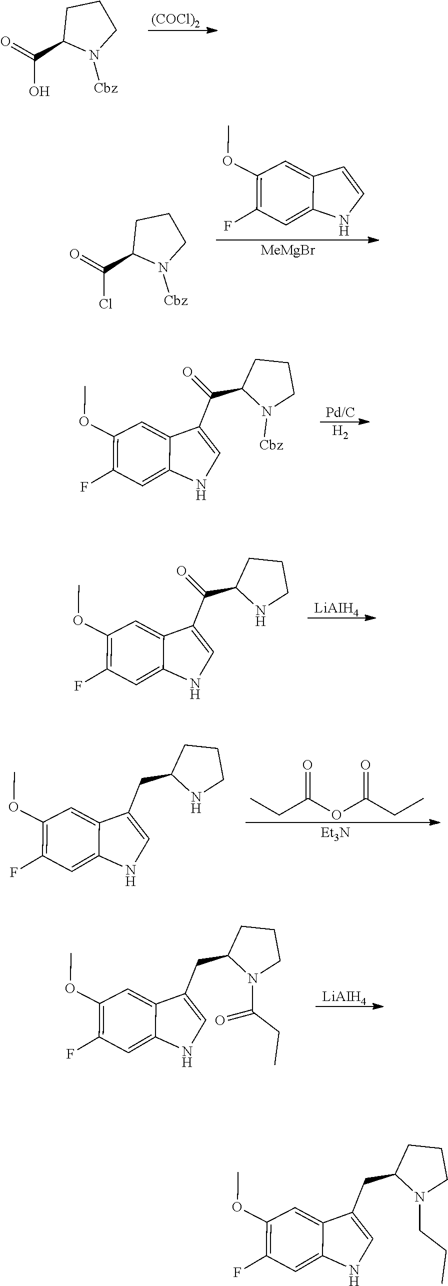

### Step 1: Benzyl (R)-2-(chlorocarbonyl)pyrrolidine-1-carboxylate

To a stirred solution of ((benzyloxy)carbonyl)-D-proline (4.00 g, 1 Eq, 16.0 mmol) in DCM (40 mL) under a nitrogen atmosphere at 0° C. was added oxalyl dichloride (7.25 g, 5.0 mL, 3.6 Eq, 57.1 mmol). The reaction mixture was stirred at 22° C. for 3.5 h. The reaction mixture was concentrated in vacuo, and the resulting crude was suspended in isohexane (3×20 mL) and dried in vacuo to provide the sub-title compound (4.30 g, 15 mmol, 94%) as a yellow oil.

m/z 220.6 (M−CO₂Cl+MeOH)⁺ (ES⁺)

### Step 2: Benzyl (R)-2-(6-fluoro-5-methoxy-1H-indole-3-carbonyl)pyrrolidine-1-carboxylate

To a stirred solution of 6-fluoro-5-methoxy-1H-indole (1.05 g, 1.8 Eq, 6.36 mmol) in toluene (10.0 mL) under a nitrogen atmosphere at 22° C. was added MeMgBr in Et₂O (0.82 g, 2.3 mL, 3.00 molar, 1.9 Eq, 6.9 mmol) dropwise over 2 minutes. THF (4 mL) was added, and the reaction mixture was stirred at rt for 4 h. The product from step 1 above (0.97 g, 1 Eq, 3.6 mmol) in dry toluene (10.0 mL) was added dropwise and the reaction mixture was stirred at rt for 18 h. The reaction mixture was quenched with sat. aq. NaHCO₃ (500 mL) and left to stir at rt for 1 h. The resulting suspension was extracted with EtOAc (3×50 mL), and the collected organic layers were dried (Na₂SO₄) and concentrated in vacuo. The crude product was purified by chromatography on silica gel (40 g cartridge, 25-100% EtOAc/isohexane) to afford the sub-title compound (766 mg, 1.6 mmol, 45%).

m/z 397.1 (M+H)+ (ES+); 395.3 (M−H)− (ES−)

1H NMR (500 MHz, DMSO) δ 11.95 (s, 1H), 8.39 (d, J=10.7 Hz, 1H), 7.85 (dd, J=15.3, 8.7 Hz, 1H), 7.40-7.36 (m, 2H), 7.36-7.31 (m, 1H), 7.17-6.99 (m, 3H), 5.25-5.15 (m, 1H), 5.09-5.01 (m, 1H), 4.94 (q, J=13.2 Hz, 1H), 3.86 (d, J=3.7 Hz, 3H), 3.56-3.45 (m, 2H), 2.46-2.27 (m, 1H), 1.91-1.80 (m, 3H).

19F NMR (471 MHz, DMSO) δ −138.29-−138.38 (m).

### Step 3: (R)-6-Fluoro-5-methoxy-3-prolyl-1H-indole

A stirred solution of the product from step 2 above (549 mg, 1 Eq, 1.38 mmol) and Pd/C (87 L) (177 mg, 5% Wt, 0.06 Eq, 83.1 μmol) in MeOH (13.0 mL) under a hydrogen atmosphere at 1 bar and 22° C. was charged with hydrogen (4.20 mg, 1.5 Eq, 2.08 mmol). The reaction mixture was stirred for 2 h. The reaction mixture was filtered through glass microfibres, rinsing with MeOH (5 mL). The solvent was removed in vacuo to afford the sub-title compound (316 mg, 1.1 mmol, 83%).

m/z 263.1 (M+H)⁺ (ES+); 260.9 (M−H)⁻ (ES−)

¹H NMR (500 MHz, DMSO) δ 11.91 (s, 1H), 8.33 (s, 1H), 7.85 (d, J=8.7 Hz, 1H), 7.32 (d, J=11.4 Hz, 1H), 4.35 (dd, J=8.6, 6.0 Hz, 1H), 3.86 (s, 3H), 3.08-3.00 (m, 1H), 2.78-2.70 (m, 1H), 2.20-2.09 (m, 1H), 1.74-1.69 (m, 1H), 1.69-1.57 (m, 3H).

¹⁹F NMR (471 MHz, DMSO) δ −138.50.

### Step 4: (R)-6-Fluoro-5-methoxy-3-(pyrrolidin-2-ylmethyl)-1H-indole

To a stirred solution of the product from step 3 above (306 mg, 1 Eq, 1.17 mmol) in THF (8.00 mL) under a nitrogen atmosphere at 0° C. was added LiAlH₄ in THF (182 mg, 2.00 mL, 2.40 molar, 4.11 Eq, 4.80 mmol). The reaction mixture was heated to 66° C. and stirred for 2 h before being cooled to 0° C. and quenched with 3:1 THF/water mixture (12 mL). The mixture was warmed to rt and stirred for 1 h before Na₂SO₄ was added and the mixture was filtered, washing with EtOAc (15 mL). The solvent was removed in vacuo to afford the sub-title compound (310 mg, 0.92 mmol, 79%, 74% Purity) as a brown gum.

A portion of the material was dissolved in DMSO (1.4 mL), filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters X-Select CSH C18 ODB prep column, 130 Å, 5 μm, 30 mm×100 mm, flow rate 40 mL min-1 eluting with a 0.1% formic acid in water-MeCN gradient over 12.5 mins using UV across all wavelengths with PDA as well as a QDA and ELS detector. At-column dilution pump gives 2 mL min-1 MeOH over the entire method, which is included in the following MeCN percentages. Gradient information: 0.0-0.5 min, 10% MeCN; 0.5-10.5 min, ramped from 10% MeCN to 25% MeCN; 10.5-10.6 min, ramped from 25% MeCN to 100% MeCN; 10.6-12.5 min, held at 100% MeCN. The clean fractions were evaporated in a Genevac to afford the first title compound (11.0 mg, 35 mol, 3.0%) as a colourless gum.

m/z 249.2 (M+H)⁺ (ES+)

¹H NMR (500 MHz, DMSO) δ 10.83 (s, 1H), 8.39 (s, 1H), 7.25 (d, J=8.4 Hz, 1H), 7.20-7.15 (m, 2H), 3.87-3.79 (m, 4H), 3.63-3.55 (m, 1H), 3.18-3.09 (m, 1H), 3.05-2.95 (m, 2H), 2.89 (dd, J=14.5, 7.3 Hz, 1H), 1.98-1.85 (m, 1H), 1.82-1.73 (m, 1H), 1.61-1.51 (m, 1H).

¹⁹F NMR (471 MHz, DMSO) δ −141.02.

### Step 5: (R)-1-(2-((6-Fluoro-5-methoxy-1H-indol-3-yl)methyl)pyrrolidin-1-yl)propan-1-one

To a stirred solution of the product from step 4 above (257 mg, 75% Wt, 1 Eq, 776 μmol) in THF (4.00 mL) was added propionic anhydride (213 mg, 210 μL, 2.11 Eq, 1.64 mmol) and Et₃N (392 mg, 540 φL, 4.99 Eq, 3.87 mmol). The reaction mixture was stirred at rt for 18 h. The mixture was diluted with EtOAc (10 m), and water (10 mL) was added before the layers were separated. The aq. layer was extracted with EtOAc (3×5 m), and the collected organic layers were dried (MgSO₄), filtered and concentrated in vacuo to afford the sub-title compound (315 mg, 0.78 mmol, 100%) as a brown gum.

m/z 304.8 (M+H)⁺ (ES+); 302.9 (M−H)⁻ (ES−)

### Step 6: (R)-6-Fluoro-5-methoxy-3-((1-propylpyrrolidin-2-yl)methyl)-1H-indole

To a stirred solution of the product from step 5 (315 mg, 1 Eq, 0.776 mmol) in 2-Me-THF (4.00 mL) under a nitrogen atmosphere at 0° C. was added 2.4 M LiAlH₄ in THF (1.30 mL, 4.02 Eq, 3.12 mmol). The reaction mixture was heated to 66° C. and stirred for 2 h. The reaction mixture was cooled to 0° C. and quenched with 3:1 THF/water (12 mL). The resulting precipitate was removed via filtration and washed with EtOAc (3×20 mL). The combined filtrate was dried (MgSO₄) and concentrated in vacuo. The crude product was purified by chromatography on silica gel (12 g cartridge, 0-100% 2% NH₄OH in 3:1 EtOAc/EtOH in isohexanes) to afford the product which was dissolved to 8.12 mg/mL in MeOH with sonication, filtered and was then separated by chiral SFC on a Waters Prep 100 with a PDA and QDa detectors, 40° C., 120 bar. The column was a Chiralpak IH, 20×250 mm, 5 μm, flow rate 65 mL/min at 10% MeOH (0.2% DEA), 90% CO₂. The clean fractions were pooled, rinsed with MeOH, and concentrated to dryness using a rotary evaporator. The residues were re-dissolved in MeOH, transferred into final vials and evaporated on a Biotage V10. The samples were then further dried in a vacuum oven at 30° C./5 mbar overnight to afford the title compound (52.0 mg, 0.17 mmol, 62%) as a brown gum.

m/z 291.2 (M+H)⁺ (ES+)

1H NMR (500 MHz, DMSO) δ 10.66 (s, 1H), 7.15-7.11 (m, 2H), 7.07 (d, J=2.3 Hz, 1H), 3.82 (s, 3H), 3.07 (ddd, J=9.6, 6.9, 3.0 Hz, 1H), 2.95 (dd, J=13.9, 3.3 Hz, 1H), 2.89-2.80 (m, 1H), 2.59-2.53 (m, 1H), 2.21-2.11 (m, 1H), 2.08 (q, J=8.6 Hz, 1H), 1.75-1.38 (m, 7H), 0.90 (t, J=7.4 Hz, 3H).

¹⁹F NMR (471 MHz, DMSO) δ −141.49.

ee=98%

(S)-6-fluoro-5-methoxy-3-((1-propylpyrrolidin-2-yl)methyl)-1H-indole (25.0 mg, 82 μmol, 30%) was also afforded as a brown gum.

m/z 291.2 (M+H)⁺ (ES+)

¹H NMR (500 MHz, DMSO) δ 10.66 (s, 1H), 7.15-7.11 (m, 2H), 7.07 (d, J=2.3 Hz, 1H), 3.83 (s, 3H), 3.11-3.04 (m, 1H), 2.95 (dd, J=14.0, 3.4 Hz, 1H), 2.85 (dt, J=11.7, 8.3 Hz, 1H), 2.60-2.54 (m, 1H), 2.20-2.12 (m, 1H), 2.09 (q, J=8.6 Hz, 1H), 1.74-1.40 (m, 7H), 0.90 (t, J=7.4 Hz, 3H).

¹⁹F NMR (471 MHz, DMSO) δ −141.48.

ee=>99%

### Example 3: Synthesis of (R)-6-Fluoro-5-methoxy-3-(pyrrolidin-2-ylmethyl)-1H-indole, Formic Acid (Compound 10A)

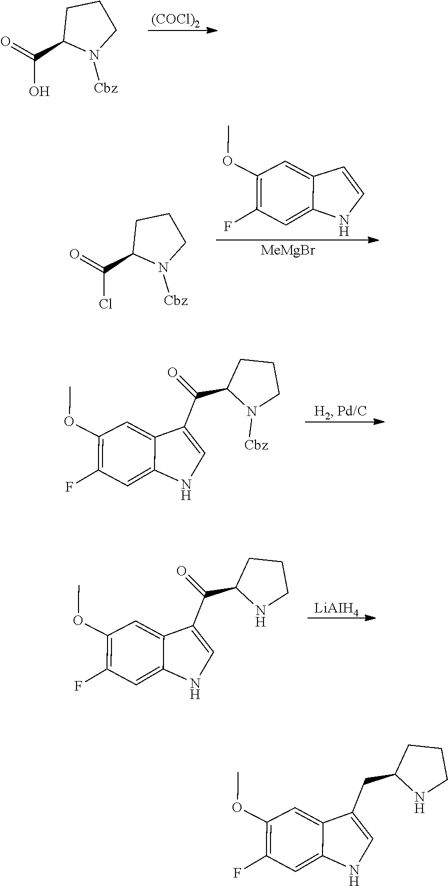

### Step 1: Benzyl (R)-2-(chlorocarbonyl)pyrrolidine-1-carboxylate

To a stirred solution of ((benzyloxy)carbonyl)-D-proline (4.00 g, 1 Eq, 16.0 mmol) in DCM (40 mL) under a nitrogen atmosphere at 0° C. was added oxalyl dichloride (7.25 g, 5.0 mL, 3.6 Eq, 57.1 mmol). The reaction mixture was stirred at 22° C. for 3.5 h. The reaction mixture was concentrated in vacuo, and the resulting crude was suspended in isohexane (3×20 mL) and dried in vacuo to provide the sub-title compound (4.30 g, 15 mmol, 94%) as a yellow oil.

m/z 220.6 (M−CO₂Cl+MeOH)⁺ (ES+)

### Step 2: Benzyl (R)-2-(6-fluoro-5-methoxy-1H-indole-3-carbonyl)pyrrolidine-1-carboxylate

To a stirred solution of the product from step 1 (1.05 g, 1.8 Eq, 6.36 mmol) in toluene (10.0 mL) under a nitrogen atmosphere at 22° C. was added MeMgBr in Et₂O (0.82 g, 2.3 mL, 3.00 molar, 1.9 Eq, 6.9 mmol) dropwise over 2 minutes. THF (4 mL) was added. The reaction mixture was stirred at rt for 4 h before the product from step 1 above (0.97 g, 1 Eq, 3.6 mmol) in toluene (10.0 mL) was added dropwise and the reaction mixture was stirred at rt for 18 h. The reaction mixture was quenched with sat. aq. NaHCO₃ (500 mL) and stirred at rt for 1 h. The resulting suspension was extracted with EtOAc (3×50 mL), and the collected organic layers were dried (Na₂SO₄) and concentrated in vacuo. The crude product was purified by chromatography on silica gel (40 g cartridge, 25-100% EtOAc/isohexane) to afford the sub-title compound (766 mg, 1.6 mmol, 45%).

m/z 397.1 (M+H)⁺ (ES+); 395.3 (M−H)⁻ (ES⁻)

¹H NMR (500 MHz, DMSO) δ 11.95 (s, 1H), 8.39 (d, J=10.7 Hz, 1H), 7.85 (dd, J=15.3, 8.7 Hz, 1H), 7.40-7.36 (m, 2H), 7.36-7.31 (m, 1H), 7.17-6.99 (m, 3H), 5.25-5.15 (m, 1H), 5.09-5.01 (m, 1H), 4.94 (q, J=13.2 Hz, 1H), 3.86 (d, J=3.7 Hz, 3H), 3.56-3.45 (m, 2H), 2.46-2.27 (m, 1H), 1.91-1.80 (m, 3H).

¹⁹F NMR (471 MHz, DMSO) δ −138.29-−138.38 (m).

### Step 3: (R)-6-Fluoro-5-methoxy-3-prolyl-1H-indole

A stirred solution of benzyl (R)-2-(6-fluoro-5-methoxy-1H-indole-3-carbonyl)pyrrolidine-1-carboxylate (549 mg, 1 Eq, 1.38 mmol) and palladium on carbon (87 L) (177 mg, 5% Wt, 0.06 Eq, 83.1 μmol) in

methanol (13.0 mL) under a hydrogen atmosphere at 1 bar and 22° C. was charged with hydrogen (4.20 mg, 1.5 Eq, 2.08 mmol). The reaction mixture was stirred for 2 hours. The reaction mixture was filtered through glass microfibres, rinsing with MeOH (5 mL). The solvent was removed in vacuo to afford the title compound (316 mg, 1.1 mmol, 83%, 95% Purity).

m/z 263.1 (M+H)⁺ (ES⁺); 260.9 (M−H)⁻ (ES⁻)

1H NMR (500 MHz, DMSO) δ 11.91 (s, 1H), 8.33 (s, 1H), 7.85 (d, J=8.7 Hz, 1H), 7.32 (d, J=11.4 Hz, 1H), 4.35 (dd, J=8.6, 6.0 Hz, 1H), 3.86 (s, 3H), 3.08-3.00 (m, 1H), 2.78-2.70 (m, 1H), 2.20-2.09 (m, 1H), 1.74-1.69 (m, 1H), 1.69-1.57 (m, 3H).

19F NMR (471 MHz, DMSO) δ −138.50.

### Step 4: (R)-6-Fluoro-5-methoxy-3-(pyrrolidin-2-ylmethyl)-1H-indole, Formic Acid

To a stirred solution of the product from step 2 (306 mg, 1 Eq, 1.17 mmol) in THF (8.00 mL) under a nitrogen atmosphere at 0° C. was added 2.4 M LiAlH₄ in THF (182 mg, 2.00 mL, 2.40 molar, 4.11 Eq, 4.80 mmol). The reaction mixture was heated to 66° C. and stirred for 2 hours before being cooled to 0° C. and quenched with 3:1 THF/water mixture (12 mL). The mixture was warmed to rt and stirred for 1 h before Na₂SO₄ was added and the mixture was filtered, washing with ethyl acetate (15 mL). The solvent was removed in vacuo before a sample was dissolved in 1.4 mL with DMSO, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters X-Select CSH C18 ODB prep column, 130 Å, 5 μm, 30 mm×100 mm, flow rate 40 mL min-1 eluting with a 0.1% Formic acid in water-MeCN gradient over 12.5 mins using UV across all wavelengths with PDA as well as a QDA and ELS detector. At-column dilution pump gives 2 mL min-1 Methanol over the entire method, which is included in the following MeCN percentages. Gradient information: 0.0-0.5 min, 10% MeCN; 0.5-10.5 min, ramped from 10% MeCN to 25% MeCN; 10.5-10.6 min, ramped from 25% MeCN to 100% MeCN; 10.6-12.5 min, held at 100% MeCN. The clean fractions were evaporated in a Genevac to afford the title compound (11.0 mg, 35 mol, 3.0%) as a colourless gum.

m/z 249.2 (M+H)⁺ (ES+); n/a (M−H)⁻ (ES⁻)

¹H NMR (500 MHz, DMSO) δ 10.83 (s, 1H), 8.39 (s, 1H), 7.25 (d, J=8.4 Hz, 1H), 7.20-7.15 (m, 2H), 3.87-3.79 (m, 4H), 3.63-3.55 (m, 1H), 3.18-3.09 (m, 1H), 3.05-2.95 (m, 2H), 2.89 (dd, J=14.5, 7.3 Hz, 1H), 1.98-1.85 (m, 1H), 1.82-1.73 (m, 1H), 1.61-1.51 (m, 1H).

¹⁹F NMR (471 MHz, DMSO) δ −141.02.

ee=43%

### Example 4: Synthesis of (R)-2-(2-((6-fluoro-5-methoxy-1H-indol-3-yl)methyl)pyrrolidin-1-yl)ethan-1-ol, Formic Acid (Compound 7B)

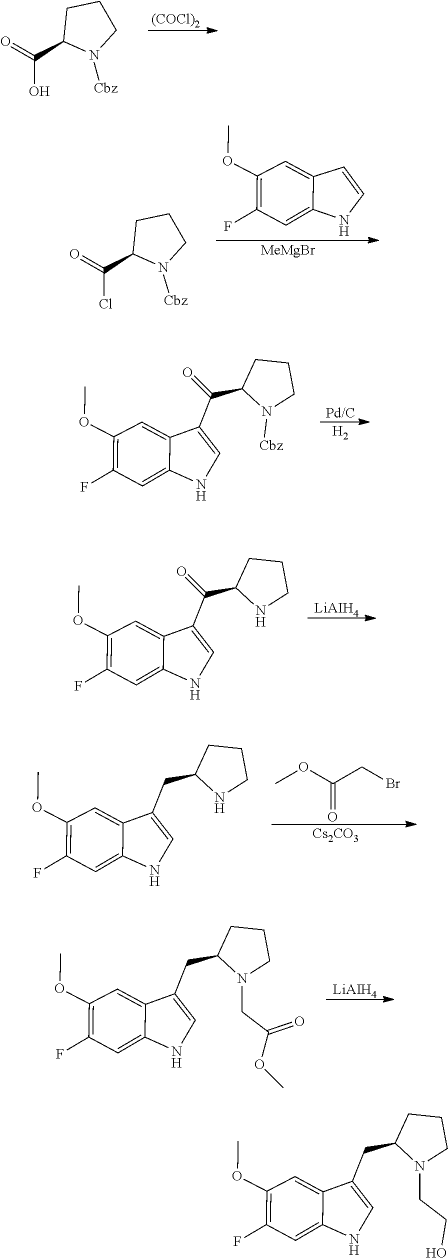

### Step 1: Benzyl (R)-2-(chlorocarbonyl)pyrrolidine-1-carboxylate

To a stirred solution of ((benzyloxy)carbonyl)-D-proline (0.90 g, 1 Eq, 3.6 mmol) in DCM (10.0 mL) under a nitrogen atmosphere at 0° C. was added oxalyl dichloride (2.3 g, 1.6 mL, 5 Eq, 18 mmol). The reaction mixture was stirred at rt for 3 h. The reaction mixture was concentrated in vacuo, and the resulting crude was suspended in iso-hexane (3×20 mL) and dried in vacuo to provide the sub-title compound (0.97 g, 3.6 mmol, 100%) as a yellow oil.

m/z 220.6 (M−CO₂Cl+MeOH)⁺ (ES⁺)

### Step 2: Benzyl (R)-2-(6-fluoro-5-methoxy-1H-indole-3-carbonyl)pyrrolidine-1-carboxylate

To a stirred solution of 6-fluoro-5-methoxy-1H-indole (1.05 g, 1.8 Eq, 6.36 mmol) in dry Toluene (10.0 mL) under a nitrogen atmosphere at rt was added MeMgBr in diethyl ether (0.82 g, 2.3 mL, 3.00 molar, 1.9 Eq, 6.9 mmol) dropwise over 2 min. THF (4 mL) was added. The reaction mixture was stirred at rt for 4 hours. A solution of product from step 1 above (0.97 g, 1 Eq, 3.6 mmol) in dry Toluene (10.0 mL) was added dropwise and the reaction mixture was stirred at room temperature for 18 h. The reaction mixture was quenched with sat. aq. NaHCO₃ (50 mL) and left to stir at rt for 1 h. The resulting suspension was extracted with EtOAc (3×50 mL), and the collected organic layers were dried over Na₂SO₄ and concentrated in vacuo. The crude product was purified by chromatography on silica gel (40 g cartridge, 25-100% EtOAc/iso-hexane) to afford sub-title compound (836 mg, 1.9 mmol, 52%).

m/z 396.9 (M+H)⁺ (ES+); 395.5 (M−H)⁻ (ES−).

¹H NMR (500 MHz, DMSO) δ 11.95 (s, 1H), 8.39 (d, J=10.7 Hz, 1H), 7.85 (dd, J=15.3, 8.7 Hz, 1H), 7.42-7.29 (m, 3H), 7.18-7.01 (m, 3H), 5.26-5.14 (m, 1H), 5.12-5.01 (m, 1H), 4.94 (q, J=13.2 Hz, 1H), 3.86 (d, J=3.8 Hz, 3H), 3.56-3.45 (m, 2H), 2.42-2.25 (m, 1H), 1.93-1.81 (m, 3H).

¹⁹F NMR (471 MHz, DMSO) δ −138.33 (d, J=25.0 Hz).

### Step 3: (R)-6-Fluoro-5-methoxy-3-prolyl-1H-indole

A stirred solution of the product from step 2 above (830 mg, 1 Eq, 1.88 mmol) and Pd/C (87 L) (240 mg, 5% Wt, 0.0598 Eq, 113 μmol) in EtOH (18.0 mL) under a nitrogen atmosphere at rt was charged with hydrogen (1 bar). The reaction mixture was stirred at rt for 2 h. The reaction mixture was filtered through glass microfibres. The solvent was removed in vacuo. The residue was dissolved in MeOH (50 mL) and Pd/C (87 L) (240 mg, 5% Wt, 0.0598 Eq, 113 μmol) was added. The mixture was subjected to a hydrogen atmosphere at 2 bar and 22° C. for 5 h. The reaction mixture was filtered through glass microfibres, rinsing with MeOH (5 mL). The solvent was removed in vacuo to afford sub-title compound (485 mg, 1.8 mmol, 93%).

m/z 263.2 (M+H)⁺ (ES+); 261.0 (M−H)⁻ (ES−)

¹H NMR (500 MHz, DMSO) δ 11.94 (s, 1H), 8.33 (d, J=1.4 Hz, 1H), 7.84 (d, J=8.7 Hz, 1H), 7.33 (d, J=11.4 Hz, 1H), 4.43-4.34 (m, 1H), 3.86 (s, 3H), 3.09-3.02 (m, 1H), 2.79-2.76 (m, 1H), 2.19-2.14 (m, 1H), 1.76-1.69 (m, 1H), 1.69-1.57 (m, 2H).

¹⁹F NMR (471 MHz, DMSO) δ −138.44 (d, J=5.7 Hz).

### Step 4: (R)-6-Fluoro-5-methoxy-3-(pyrrolidin-2-ylmethyl)-1H-indole

To a stirred solution of the product from step 4 above (485 mg, 1 Eq, 1.85 mmol) in dry Tetrahydrofuran (13.00 mL) under a nitrogen atmosphere at 0° C. was added LiAlH4 in THF (281 mg, 3.70 mL, 2.00 molar, 4 Eq, 7.40 mmol). The reaction mixture was heated to 66° C. and stirred for 2 h. The reaction mixture was cooled to 0° C. and quenched with 3:1 THF/water (16 mL). The mixture was warmed to rt and stirred for 1 h before Na₂SO₄ was added and the mixture was stirred for 18 h. The suspension was filtered, and the solvent was removed in vacuo to afford sub-title compound (457 mg, 1.6 mmol, 85%) as a brown gum.

m/z 249.2 (M+H)⁺ (ES+)

### Step 5: Methyl (R)-2-(2-((6-fluoro-5-methoxy-1H-indol-3-yl)methyl)pyrrolidin-1-yl)acetate

To a stirred solution of the product from step 4 above (75.0 mg, 1 Eq, 257 μmol) and cesium carbonate (167 mg, 2 Eq, 513 μmol) in dry THF (2.00 mL) at 22° C. was added methyl 2-bromoacetate (78.6 mg, 48.6 μL, 2 Eq, 513 μmol). The reaction mixture was stirred for 18 h. The reaction mixture was quenched with sat. aq. NaHCO₃ (10 mL). The resulting aqueous suspension was extracted with EtOAc (2×10 mL). The combined organic layers were dried over MgSO₄, filtered and concentrated in vacuo. The crude product was purified by chromatography on silica gel (12 g cartridge, 0-50% (2% NH₄OH in 3:1 EtOH/EtOAc) in iso-hexane) to afford sub-title compound (44.0 mg, 0.11 mmol, 43%) as a yellow oil.

m/z 321.2 (M+H)⁺ (ES+)

¹H NMR (500 MHz, DMSO) δ 10.66 (s, 1H), 7.17 (d, J=8.5 Hz, 1H), 7.12 (d, J=11.8 Hz, 1H), 7.07 (d, J=2.4 Hz, 1H), 3.83 (s, 3H), 3.72-3.64 (m, 2H), 3.62 (s, 3H), 3.10-3.04 (m, 1H), 2.93 (dd, J=14.0, 4.0 Hz, 1H), 2.90-2.83 (m, 1H), 2.47 (d, J=8.7 Hz, 1H), 2.41 (q, J=8.3 Hz, 1H), 1.76-1.67 (m, 1H), 1.67-1.56 (m, 2H), 1.51-1.40 (m, 1H).

¹⁹F NMR (471 MHz, DMSO) δ −141.47.

### Step 6: (R)-2-(2-((6-fluoro-5-methoxy-1H-indol-3-yl)methyl)pyrrolidin-1-yl)ethan-1-ol, Formic Acid

To a stirred solution of product from step 5 above (44.0 mg, 80% Wt, 1 Eq, 110 μmol) in 2-MeTHF (2.00 mL) under a nitrogen atmosphere at 0° C. was added LiAlH₄ in THF (12.5 mg, 137 μL, 2.4 molar, 3 Eq, 330 μmol). The reaction mixture was stirred at rt for 30 min. The reaction mixture was cooled to 0° C. and quenched by slow addition of 3:1 THF/H₂O (5 mL). The mixture was stirred for 30 min at rt. Na₂SO₄ (ca 3 g) and EtOAc (15 mL) were added to the reaction mixture and stirred vigorously for 5 min. The suspension was filtered, and the filtrate was concentrated in vacuo. The crude product was purified by chromatography on silica gel (4 g cartridge, 0-100% (2% NH₄OH in 3:1 EtOAc/EtOH) in iso-hexane). The crude product was dissolved in 1.2 mL with DMSO, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge BEH C18 ODB prep column, 130 Å, 5 μm, 30 mm×100 mm, flow rate 40 mL min⁻¹ eluting with a 0.3% Ammonia in water-MeCN gradient over 17.5 mins using UV across all wavelengths with PDA as well as a QDA and ELS detector. At-column dilution pump gives 2 mL min⁻¹ Methanol over the entire method, which is included in the following MeCN percentages. Gradient information: 0.0-0.5 min, 10% MeCN; 0.5-15.5 min, ramped from 10% MeCN to 40% MeCN; 15.5-15.6 min, ramped from 40% MeCN to 100% MeCN; 15.6-17.5 min, held at 100% MeCN. The clean fractions were evaporated in a Genevac to afford the title product (13.0 mg, 38 mol, 35%) as a colourless gum.

m/z 293.2 (M+H)⁺ (ES+)

¹H NMR (500 MHz, DMSO) δ 10.68 (s, 1H), 8.21 (s, 1H), 7.16 (d, J=8.5 Hz, 1H), 7.13 (d, J=11.8 Hz, 1H), 7.09 (d, J=2.4 Hz, 1H), 3.83 (s, 3H), 3.55 (ddt, J=10.8, 7.7, 3.7 Hz, 2H), 3.18-3.10 (m, 1H), 3.04 (dt, J=12.1, 6.9 Hz, 1H), 2.98 (dd, J=14.2, 3.7 Hz, 1H), 2.79-2.69 (m, 1H), 2.57-2.52 (m, 1H), 2.40 (dt, J=12.0, 6.0 Hz, 1H), 2.27 (q, J=8.6 Hz, 1H), 1.75-1.65 (m, 1H), 1.65-1.58 (m, 2H), 1.49-1.40 (m, 1H). 2 Exchangeable protons not observed.

19F NMR (471 MHz, DMSO) δ −141.44.

### Example 5: Synthesis of (R)-6-fluoro-5-methoxy-3-((1-(pyridin-4-ylmethyl)pyrrolidin-2-yl)methyl)-1H-indole (Compound 8B)

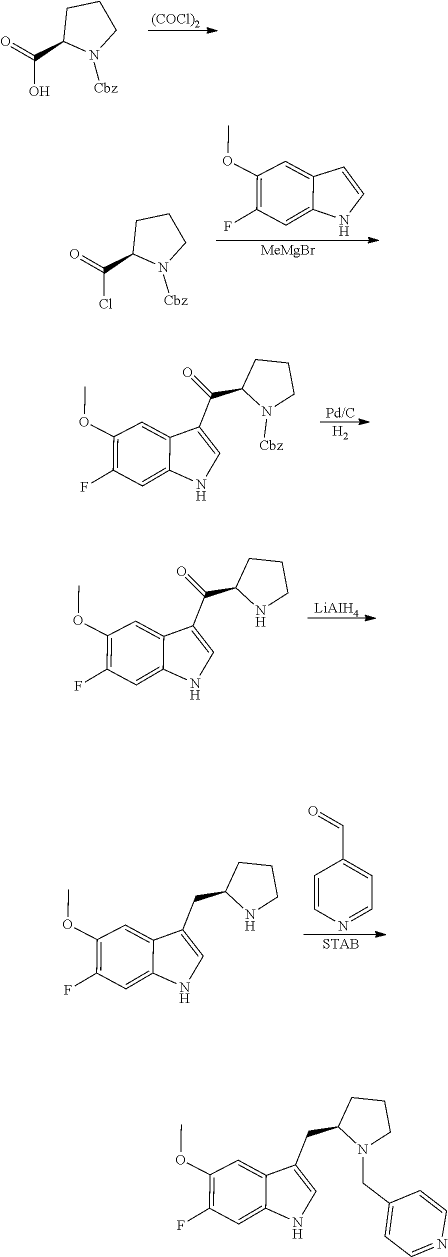

### Step 1: Benzyl (R)-2-(chlorocarbonyl)pyrrolidine-1-carboxylate

To a stirred solution of ((benzyloxy)carbonyl)-D-proline (1.32 g, 1 Eq, 5.30 mmol) in DCM (15.0 mL) under a nitrogen atmosphere at 0° C. was added oxalyl dichloride (3.33 g, 2.30 mL, 4.96 Eq, 26.3 mmol). The reaction mixture was stirred at rt for 3 h. The reaction mixture was concentrated in vacuo, and the resulting crude was suspended in iso-hexane (3×15 mL) and dried in vacuo to provide the sub-title compound (1.42 g, 5.30 mmol, 100%) as a pale-yellow oil.

m/z 264.2 (M−Cl+MeOH)⁺ (ES⁺)

### Step 2: Benzyl (R)-2-(6-fluoro-5-methoxy-1H-indole-3-carbonyl)pyrrolidine-1-carboxylate

To a stirred solution of 6-fluoro-5-methoxy-1H-indole (1.75 g, 2 Eq, 10.6 mmol) in dry Toluene (10.0 mL) under a nitrogen atmosphere at rt was added MeMgBr in diethyl ether (1.26 g, 3.54 mL, 3.00 molar, 2 Eq, 10.6 mmol) dropwise over 2 min. THF (4 mL) was added. The reaction mixture was stirred at rt for 4 h. A solution of product from step 1 above (1.42 g, 1 Eq, 5.30 mmol) in dry Toluene (15.0 mL) was added dropwise and the reaction mixture was stirred at rt for 72 h. The reaction mixture was quenched with sat. aq. NaHCO₃ (50 mL) and left to stir at rt for 1 h. The resulting suspension was extracted with EtOAc (3×50 mL), and the collected organic layers were dried over Na₂SO₄ and concentrated in vacuo. The crude product was purified by chromatography on silica gel (80 g cartridge, 25-100% EtOAc/iso-hexane) to afford sub-title compound (1.32 g, 3.2 mmol, 60%, 95% Purity).

m/z 397.1 (M+H)⁺ (ES+); 395.6 (M−H)⁻ (ES−)

¹H NMR (500 MHz, DMSO) δ 11.95 (s, 1H), 8.39 (d, J=10.6 Hz, 1H), 7.89-7.80 (m, 1H), 7.42-7.31 (m, 3H), 7.17-7.00 (m, 3H), 5.24-5.12 (m, 1H), 5.10-5.00 (m, 1H), 4.94 (q, J=13.2 Hz, 1H), 3.86 (s, 3H), 3.56-3.47 (m, 2H), 2.42-2.28 (m, 2H), 1.91-1.80 (m, 2H).

¹⁹F NMR (471 MHz, DMSO) δ −138.33 (m).

### Step 3: (R)-6-Fluoro-5-methoxy-3-prolyl-1H-indole

A stirred solution of the product from step 2 above (1.32 g, 1 Eq, 3.33 mmol) and Pd/C (87 L) (354 mg, 5% Wt, 0.05 Eq, 166 μmol) in MeOH (100.0 mL) under a nitrogen atmosphere at rt was charged with hydrogen (1 bar). The reaction mixture was stirred at rt for 18 h. The reaction mixture was filtered through glass microfibres, rinsing with MeOH (5 mL). The solvent was removed in vacuo to afford sub-title compound (840 mg, 3.0 mmol, 91%).

m/z 263.5 (M+H)⁺ (ES+); 261.0 (M−H)⁻ (ES−)

¹H NMR (500 MHz, DMSO) δ 8.34 (s, 1H), 7.84 (d, J=8.7 Hz, 1H), 7.33 (d, J=11.4 Hz, 1H), 4.40 (dd, J=8.7, 6.0 Hz, 1H), 3.86 (s, 3H), 3.06 (dt, J=11.6, 6.2 Hz, 1H), 2.78 (dt, J=10.4, 6.5 Hz, 1H), 2.22-2.11 (m, 1H), 1.77-1.69 (m, 1H), 1.69-1.59 (m, 2H). Two exchangeable protons not observed.

¹⁹F NMR (471 MHz, DMSO) δ −138.43.

### Step 4: (R)-6-Fluoro-5-methoxy-3-(pyrrolidin-2-ylmethyl)-1H-indole

To a stirred suspension of the product from step 3 above (835 mg, 1 Eq, 3.18 mmol) in dry THF (30.0 mL) under a nitrogen atmosphere at 0° C. was added LiAlH₄ in THF (6.50 mL, 2.00 molar, 4.08 Eq, 13.0 mmol). The reaction mixture was heated to 66° C. and stirred for 2 h. The reaction mixture was cooled to 0° C. and quenched with 3:1 THF/water (16 mL). The mixture was warmed to rt and stirred for 1 h before being filtered. Na₂SO₄ was added to the filtrate and the mixture was filtered and concentrated in vacuo to afford sub-title compound (750 mg, 1.9 mmol, 61%) as a brown gum.

m/z 249.5 (M+H)⁺ (ES+)

### Step 5: (R)-6-fluoro-5-methoxy-3-((1-(pyridin-4-ylmethyl)pyrrolidin-2-yl)methyl)-1H-indole

To a stirred solution of the product from step 4 above (100 mg, 1 Eq, 258 μmol) in dry THF (2.00 mL) at 22° C. was added isonicotinaldehyde (33.1 mg, 29.1 μL, 1.2 Eq, 309 μmol). The reaction mixture was stirred for 1 h. Sodium triacetoxyhydroborate (164 mg, 3 Eq, 773 μmol) was added and the reaction mixture left to stir at rt over the weekend.

The reaction mixture was quenched with sat. aq. NaHCO₃ (5 mL) and left to stir for 1 h. The aqueous suspension was diluted with water (5 mL) and extracted with EtOAc (3×15 mL), and the collected organic layers was washed with brine (30 mL), dried over Na₂SO₄, filtered and concentrated in vacuo.

The crude material was dissolved in 2.2 mL with DMSO, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters X-Select CSH C18 ODB prep column, 130 Å, 5 μm, 30 mm×100 mm, flow rate 40 mL min−1 eluting with a 0.1% Formic acid in water-MeCN gradient over 12.5 mins using UV across all wavelengths with PDA as well as a QDA and ELS detector. At-column dilution pump gives 2 mL min-1 Methanol over the entire method, which is included in the following MeCN percentages. Gradient information: 0.0-0.5 min, 10% MeCN; 0.5-10.5 min, ramped from 10% MeCN to 20% MeCN; 10.5-10.6 min, ramped from 20% MeCN to 100% MeCN; 10.6-12.5 min, held at 100% MeCN. The clean fractions were evaporated in a Genevac to afford title compound (43.0 mg, 0.12 mmol, 48%) as a pale-yellow gum.

m/z 340.3 (M+H)⁺ (ES+)

¹H NMR (500 MHz, DMSO) δ 10.68 (s, 1H), 8.54-8.46 (m, 2H), 8.15 (s, 1H), 7.40-7.34 (m, 2H), 7.13 (d, J=12.0 Hz, 1H), 7.11-7.08 (m, 1H), 4.16 (d, J=14.2 Hz, 1H), 3.80 (s, 3H), 3.35 (d, J=14.2 Hz, 1H), 3.02 (dd, J=14.1, 4.0 Hz, 1H), 2.86 (ddd, J=9.8, 7.1, 3.2 Hz, 1H), 2.82-2.75 (m, 1H), 2.60 (dd, J=14.2, 8.9 Hz, 1H), 2.16 (q, J=8.6 Hz, 1H), 1.82-1.72 (m, 1H), 1.69-1.58 (m, 2H), 1.58-1.48 (m, 1H).

The product was dissolved in MeOH to 1 mg/mL and screened by SFC (Waters UPC2, with a PDA and a QDA detector). The optimal conditions are using a Chiralpak IG column, 4.6×250 mm, 5 μm, with mobile phase 35% Methanol (28 mM NH3), 65% CO2. The flow rate was 4 mL/min. The run time was 4.0 min.

ee=50.42%

### Example 6: Synthesis of (R)-6-fluoro-5-methoxy-3-((1-((3-methoxypyridin-4-yl)methyl)pyrrolidin-2-yl)methyl)-1H-indole, Formic Acid (Compound 11A)

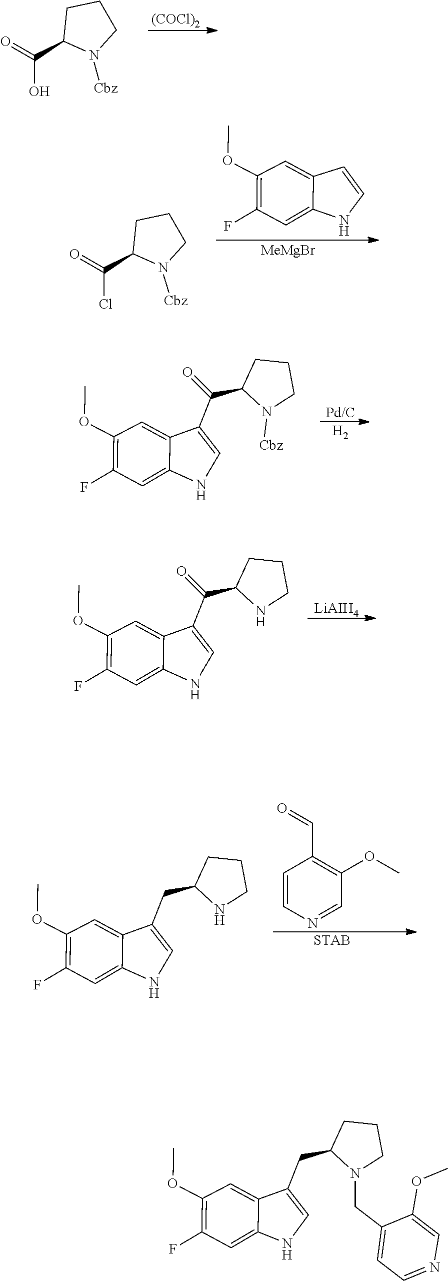

### Step 1: Benzyl (R)-2-(chlorocarbonyl)pyrrolidine-1-carboxylate

To a stirred solution of ((benzyloxy)carbonyl)-D-proline (1.32 g, 1 Eq, 5.30 mmol) in DCM (15.0 mL) under a nitrogen atmosphere at 0° C. was added oxalyl dichloride (3.33 g, 2.30 mL, 4.96 Eq, 26.3 mmol). The reaction mixture was stirred at rt for 3 h. The reaction mixture was concentrated in vacuo, and the resulting crude was suspended in iso-hexane (3×15 mL) and dried in vacuo to provide the sub-title compound (1.42 g, 5.30 mmol, 100%) as a pale-yellow oil.

m/z 264.2 (M−Cl+MeOH)⁺ (ES⁺)

### Step 2: Benzyl (R)-2-(6-fluoro-5-methoxy-1H-indole-3-carbonyl)pyrrolidine-1-carboxylate

To a stirred solution of 6-fluoro-5-methoxy-1H-indole (1.75 g, 2 Eq, 10.6 mmol) in dry Toluene (10.0 mL) under a nitrogen atmosphere at rt was added MeMgBr in diethyl ether (1.26 g, 3.54 mL, 3.00 molar, 2 Eq, 10.6 mmol) dropwise over 2 min. THF (4 mL) was added. The reaction mixture was stirred at rt for 4 h. A solution of product from step 1 above (1.42 g, 1 Eq, 5.30 mmol) in dry Toluene (15.0 mL) was added dropwise and the reaction mixture was stirred at rt for 72 h. The reaction mixture was quenched with sat. aq. NaHCO₃ (50 mL) and left to stir at rt for 1 h. The resulting suspension was extracted with EtOAc (3×50 mL), and the collected organic layers were dried over Na₂SO₄ and concentrated in vacuo. The crude product was purified by chromatography on silica gel (80 g cartridge, 25-100% EtOAc/iso-hexane) to afford sub-title compound (1.32 g, 3.2 mmol, 60%, 95% Purity).

m/z 397.1 (M+H)⁺ (ES+); 395.6 (M−H)⁻ (ES−)

¹H NMR (500 MHz, DMSO) δ 11.95 (s, 1H), 8.39 (d, J=10.6 Hz, 1H), 7.89-7.80 (m, 1H), 7.42-7.31 (m, 3H), 7.17-7.00 (m, 3H), 5.24-5.12 (m, 1H), 5.10-5.00 (m, 1H), 4.94 (q, J=13.2 Hz, 1H), 3.86 (s, 3H), 3.56-3.47 (m, 2H), 2.42-2.28 (m, 2H), 1.91-1.80 (m, 2H).

¹⁹F NMR (471 MHz, DMSO) δ −138.33 (m).

### Step 3: (R)-6-Fluoro-5-methoxy-3-prolyl-1H-indole

A stirred solution of the product from step 2 above (1.32 g, 1 Eq, 3.33 mmol) and Pd/C (87 L) (354 mg, 5% Wt, 0.05 Eq, 166 μmol) in MeOH (100.0 mL) under a nitrogen atmosphere at rt was charged with hydrogen (1 bar). The reaction mixture was stirred at rt for 18 h. The reaction mixture was filtered through glass microfibres, rinsing with MeOH (5 mL). The solvent was removed in vacuo to afford sub-title compound (840 mg, 3.0 mmol, 91%).

m/z 263.5 (M+H)⁺ (ES+); 261.0 (M−H)⁻ (ES−)

¹H NMR (500 MHz, DMSO) δ 8.34 (s, 1H), 7.84 (d, J=8.7 Hz, 1H), 7.33 (d, J=11.4 Hz, 1H), 4.40 (dd, J=8.7, 6.0 Hz, 1H), 3.86 (s, 3H), 3.06 (dt, J=11.6, 6.2 Hz, 1H), 2.78 (dt, J=10.4, 6.5 Hz, 1H), 2.22-2.11 (m, 1H), 1.77-1.69 (m, 1H), 1.69-1.59 (m, 2H). Two exchangeable protons not observed.

¹⁹F NMR (471 MHz, DMSO) δ −138.43.

### Step 4: (R)-6-Fluoro-5-methoxy-3-(pyrrolidin-2-ylmethyl)-1H-indole

To a stirred suspension of the product from step 3 above (835 mg, 1 Eq, 3.18 mmol) in dry THF (30.0 mL) under a nitrogen atmosphere at 0° C. was added LiAlH₄ in THF (6.50 mL, 2.00 molar, 4.08 Eq, 13.0 mmol). The reaction mixture was heated to 66° C. and stirred for 2 h. The reaction mixture was cooled to 0° C. and quenched with 3:1 THF/water (16 mL). The mixture was warmed to rt and stirred for 1 h before being filtered. Na₂SO₄ was added to the filtrate and the mixture was filtered and concentrated in vacuo to afford sub-title compound (750 mg, 1.9 mmol, 61%) as a brown gum.

m/z 249.5 (M+H)⁺ (ES+)

### Step 5: (R)-6-fluoro-5-methoxy-3-((1-((3-methoxypyridin-4-yl)methyl)pyrrolidin-2-yl)methyl)-1H-indole, Formic Acid

To a stirred solution of the product from step 4 above (100 mg, 1 Eq, 258 μmol) in dry THF (2.00 mL) at 22° C. was added 3-methoxyisonicotinaldehyde (42.4 mg, 1.2 Eq, 309 μmol). The reaction mixture was stirred for 1 h. Sodium triacetoxyhydroborate (164 mg, 3 Eq, 773 μmol) was added and the reaction mixture left to stir at rt over the weekend. The reaction mixture was quenched with sat. aq. NaHCO₃ (5 mL) and left to stir for 1 h. The aqueous suspension was diluted with water (5 mL) and extracted with EtOAc (3×15 mL), and the collected organic layers was washed with brine (30 mL), dried over Na₂SO₄, filtered and concentrated in vacuo. The crude material was dissolved in 2.1 mL with DMSO, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters X-Select CSH C18 ODB prep column, 130 Å, 5 μm, 30 mm×100 mm, flow rate 40 mL min−1 eluting with a 0.1% Formic acid in water-MeCN gradient over 17.5 mins using UV across all wavelengths with PDA as well as a QDA and ELS detector. At-column dilution pump gives 2 mL min−1 Methanol over the entire method, which is included in the following MeCN percentages. Gradient information: 0.0-0.5 min, 10% MeCN; 0.5-15.5 min, ramped from 10% MeCN to 15% MeCN; 15.5-15.6 min, ramped from 15% MeCN to 100% MeCN; 15.6-17.5 min, held at 100% MeCN. The clean fractions were evaporated in a Genevac to afford title compound (39.8 mg, 93 mol, 36%) as an orange-brown gum.

m/z 370.3 (M+H)⁺ (ES+)

¹H NMR (500 MHz, DMSO) δ 10.67 (s, 1H), 8.30 (s, 1H), 8.19 (d, J=4.7 Hz, 1H), 8.15 (s, 1H), 7.40 (d, J=4.7 Hz, 1H), 7.15-7.07 (m, 3H), 4.01 (d, J=15.3 Hz, 1H), 3.89 (s, 3H), 3.79 (s, 3H), 3.49 (d, J=15.2 Hz, 1H), 3.00 (dd, J=14.2, 3.9 Hz, 1H), 2.95 (ddd, J=9.9, 7.3, 3.1 Hz, 1H), 2.86-2.79 (m, 1H), 2.59 (dd, J=14.2, 8.8 Hz, 1H), 2.16 (q, J=8.6 Hz, 1H), 1.81-1.72 (m, 1H), 1.62 (p, J=8.2 Hz, 2H), 1.52 (td, J 10.9, 6.0 Hz, 1H). One exchangeable proton not observed—Formate salt.

¹⁹F NMR (471 MHz, DMSO) δ −141.45.

The product was dissolved in MeOH to 1 mg/mL and screened by SFC (Waters UPC2, with a PDA and a QDA detector). The optimal conditions are using a Chiralpak IG column, 4.6×250 mm, 5 μm, with mobile phase 35% Methanol (28 mM NH3), 65% CO2. The flow rate was 4 mL/min. The run time was 4.0 min.

ee=50.34%.

### Example 7: Synthesis of (R)-1-(2-((6-fluoro-5-methoxy-1H-indol-3-yl)methyl)pyrrolidin-1-yl)-2-methylpropan-2-ol (Compound 22B)

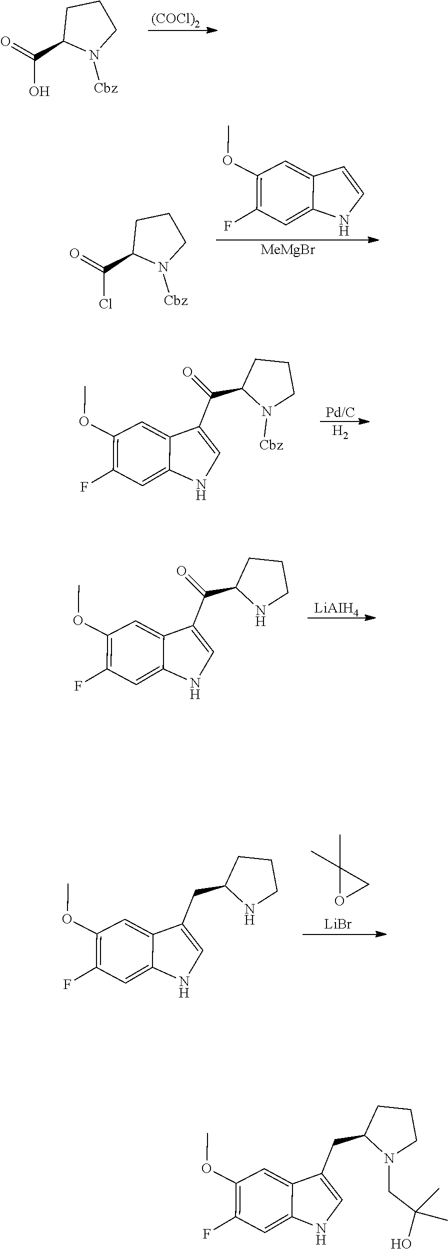

### Step 1: Benzyl (R)-2-(chlorocarbonyl)pyrrolidine-1-carboxylate

To a stirred solution of ((benzyloxy)carbonyl)-D-proline (0.90 g, 1 Eq, 3.6 mmol) in DCM (10.0 mL) under a nitrogen atmosphere at 0° C. was added oxalyl dichloride (2.3 g, 1.6 mL, 5 Eq, 18 mmol). The reaction mixture was stirred at rt for 3 h. The reaction mixture was concentrated in vacuo, and the resulting crude was suspended in iso-hexane (3×20 mL) and dried in vacuo to provide the sub-title compound (0.97 g, 3.6 mmol, 100%) as a yellow oil.

m/z 220.6 (M−CO₂Cl+MeOH)⁺ (ES⁺)

### Step 2: Benzyl (R)-2-(6-fluoro-5-methoxy-1H-indole-3-carbonyl)pyrrolidine-1-carboxylate

To a stirred solution of 6-fluoro-5-methoxy-1H-indole (1.05 g, 1.8 Eq, 6.36 mmol) in dry Toluene (10.0 mL) under a nitrogen atmosphere at rt was added MeMgBr in diethyl ether (0.82 g, 2.3 mL, 3.00 molar, 1.9 Eq, 6.9 mmol) dropwise over 2 min. THF (4 mL) was added. The reaction mixture was stirred at rt for 4 hours. A solution of product from step 1 above (0.97 g, 1 Eq, 3.6 mmol) in dry Toluene (10.0 mL) was added dropwise and the reaction mixture was stirred at room temperature for 18 h. The reaction mixture was quenched with sat. aq. NaHCO₃ (50 mL) and left to stir at rt for 1 h. The resulting suspension was extracted with EtOAc (3×50 mL), and the collected organic layers were dried over Na₂SO₄ and concentrated in vacuo. The crude product was purified by chromatography on silica gel (40 g cartridge, 25-100% EtOAc/iso-hexane) to afford sub-title compound (836 mg, 1.9 mmol, 52%).

m/z 396.9 (M+H)⁺ (ES+); 395.5 (M−H)⁻ (ES−).

¹H NMR (500 MHz, DMSO) δ 11.95 (s, 1H), 8.39 (d, J=10.7 Hz, 1H), 7.85 (dd, J=15.3, 8.7 Hz, 1H), 7.42-7.29 (m, 3H), 7.18-7.01 (m, 3H), 5.26-5.14 (m, 1H), 5.12-5.01 (m, 1H), 4.94 (q, J=13.2 Hz, 1H), 3.86 (d, J=3.8 Hz, 3H), 3.56-3.45 (m, 2H), 2.42-2.25 (m, 1H), 1.93-1.81 (m, 3H).

¹⁹F NMR (471 MHz, DMSO) δ −138.33 (d, J=25.0 Hz).

### Step 3: (R)-6-Fluoro-5-methoxy-3-prolyl-1H-indole

A stirred solution of the product from step 2 above (830 mg, 1 Eq, 1.88 mmol) and Pd/C (87 L) (240 mg, 5% Wt, 0.0598 Eq, 113 μmol) in EtOH (18.0 mL) under a nitrogen atmosphere at rt was charged with hydrogen (1 bar). The reaction mixture was stirred at rt for 2 h. The reaction mixture was filtered through glass microfibres. The solvent was removed in vacuo. The residue was dissolved in MeOH (50 mL) and Pd/C (87 L) (240 mg, 5% Wt, 0.0598 Eq, 113 μmol) was added. The mixture was subjected to a hydrogen atmosphere at 2 bar and 22° C. for 5 h. The reaction mixture was filtered through glass microfibres, rinsing with MeOH (5 mL). The solvent was removed in vacuo to afford sub-title compound (485 mg, 1.8 mmol, 93%).

m/z 263.2 (M+H)⁺ (ES+); 261.0 (M−H)⁻ (ES−)

¹H NMR (500 MHz, DMSO) δ 11.94 (s, 1H), 8.33 (d, J=1.4 Hz, 1H), 7.84 (d, J=8.7 Hz, 1H), 7.33 (d, J=11.4 Hz, 1H), 4.43-4.34 (m, 1H), 3.86 (s, 3H), 3.09-3.02 (m, 1H), 2.79-2.76 (m, 1H), 2.19-2.14 (m, 1H), 1.76-1.69 (m, 1H), 1.69-1.57 (m, 2H).

¹⁹F NMR (471 MHz, DMSO) δ −138.44 (d, J=5.7 Hz).

### Step 4: (R)-6-Fluoro-5-methoxy-3-(pyrrolidin-2-ylmethyl)-1H-indole

To a stirred solution of the product from step 4 above (485 mg, 1 Eq, 1.85 mmol) in dry Tetrahydrofuran (13.00 mL) under a nitrogen atmosphere at 0° C. was added LiAlH4 in THF (281 mg, 3.70 mL, 2.00 molar, 4 Eq, 7.40 mmol). The reaction mixture was heated to 66° C. and stirred for 2 h. The reaction mixture was cooled to 0° C. and quenched with 3:1 THF/water (16 mL). The mixture was warmed to rt and stirred for 1 h before Na₂SO₄ was added and the mixture was stirred for 18 h. The suspension was filtered, and the solvent was removed in vacuo to afford sub-title compound (457 mg, 1.6 mmol, 85%) as a brown gum.

m/z 249.2 (M+H)⁺ (ES+)

### Step 5: (R)-6-fluoro-5-methoxy-3-((1-(oxetan-3-yl)pyrrolidin-2-yl)methyl)-1H-indole

To a stirred solution of the product from step 4 above (100 mg, 1 Eq, 258 μmol) in dry MeOH (2.50 mL) under a nitrogen atmosphere at rt was added 1,2-Epoxy-isobutane (115 μL, 5 Eq, 1.29 mmol) and lithium bromide (56.0 mg, 2.5 Eq, 644 μmol). The reaction mixture was stirred for 18 h at 60° C., and then 48 h at rt. The reaction mixture was diluted with distilled water (10 mL) and extracted into EtOAc (3×10 mL). The combined organic layers were collected, dried over Na₂SO₄, filtered and concentrated in vacuo. The crude material was combined with a previous crude batch (20.0 mg, 85% Wt, 53.1 μmol). The combined crude material was dissolved in 2.3 mL with DMSO, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge BEH C18 ODB prep column, 130 Å, 5 μm, 30 mm×100 mm, flow rate 40 mL min−1 eluting with a 0.3% Ammonia in water-MeCN gradient over 17.5 mins using UV across all wavelengths with PDA as well as a QDA and ELS detector. At-column dilution pump gives 2 mL min-1 Methanol over the entire method, which is included in the following MeCN percentages. Gradient information: 0.0-0.5 min, 25% MeCN; 0.5-15.5 min, ramped from 25% MeCN to 55% MeCN; 15.5-15.6 min, ramped from 55% MeCN to 100% MeCN; 15.6-17.5 min, held at 100% MeCN. The clean fractions were evaporated in a freeze-dryer to afford title compound (56.7 mg, 0.18 mmol, 56%) as an orange gum.

m/z 321.2 (M+H)⁺

¹H NMR (500 MHz, DMSO) δ 10.66 (s, 1H), 7.14 (d, J=2.2 Hz, 1H), 7.12-7.11 (m, 1H), 7.08 (d, J=2.3 Hz, 1H), 4.03 (s, 1H), 3.83 (s, 3H), 3.30-3.24 (m, 1H), 2.96 (dd, J 14.2, 3.5 Hz, 1H), 2.77 (d, J=13.1 Hz, 1H), 2.73-2.66 (m, 1H), 2.44 (dd, J=14.1, 9.7 Hz, 1H), 2.32-2.23 (m, 2H), 1.70-1.53 (m, 3H), 1.44-1.34 (m, 1H), 1.14 (d, J=1.7 Hz, 6H).

The product was dissolved in MeOH to 1 mg/mL and screened by SFC (Waters UPC2, with a PDA and a QDA detector). The optimal conditions are using a Chiralpak IG, 4.6×250 mm, 5 μm, with mobile phase 30% MeOH (28 mM NH₃), 70% CO₂. The flow rate was 4 mL/min. The run time was 3.5 mins.

ee=50.88%

### Example 8: Synthesis of (R)-3-((1-ethylpyrrolidin-2-yl)methyl)-6-fluoro-5-methoxy-1H-indole, Formic Acid (Compound 20B)

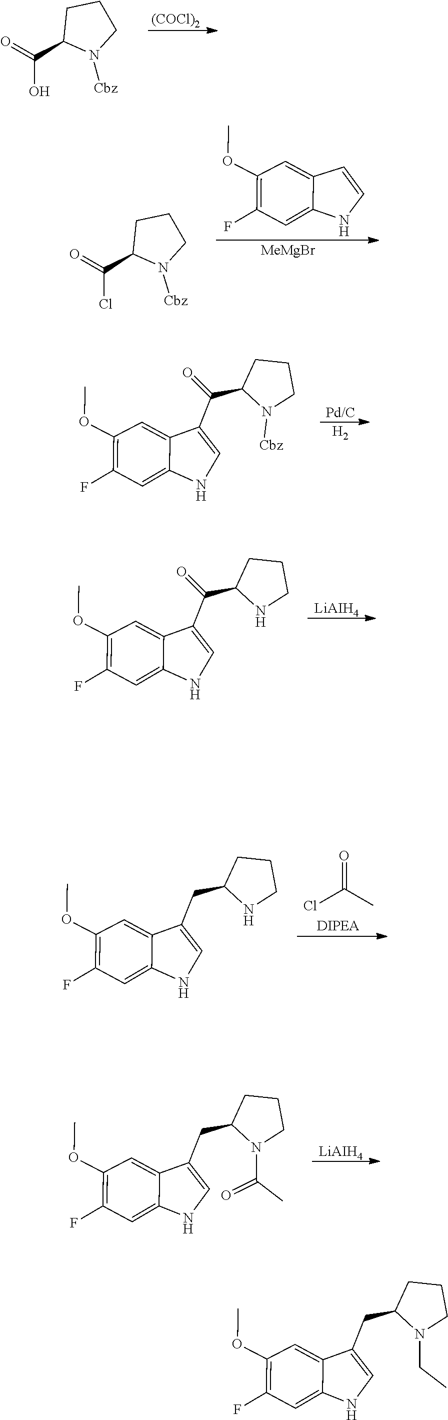

### Step 1: Benzyl (R)-2-(chlorocarbonyl)pyrrolidine-1-carboxylate

To a stirred solution of ((benzyloxy)carbonyl)-D-proline (0.90 g, 1 Eq, 3.6 mmol) in DCM (10.0 mL) under a nitrogen atmosphere at 0° C. was added oxalyl dichloride (2.3 g, 1.6 mL, 5 Eq, 18 mmol). The reaction mixture was stirred at rt for 3 h. The reaction mixture was concentrated in vacuo, and the resulting crude was suspended in iso-hexane (3×20 mL) and dried in vacuo to provide the sub-title compound (0.97 g, 3.6 mmol, 100%) as a yellow oil.

m/z 220.6 (M−CO₂Cl+MeOH)⁺ (ES⁺)

### Step 2: Benzyl (R)-2-(6-fluoro-5-methoxy-1H-indole-3-carbonyl)pyrrolidine-1-carboxylate

To a stirred solution of 6-fluoro-5-methoxy-1H-indole (1.05 g, 1.8 Eq, 6.36 mmol) in dry Toluene (10.0 mL) under a nitrogen atmosphere at rt was added MeMgBr in diethyl ether (0.82 g, 2.3 mL, 3.00 molar, 1.9 Eq, 6.9 mmol) dropwise over 2 min. THF (4 mL) was added. The reaction mixture was stirred at rt for 4 hours. A solution of product from step 1 above (0.97 g, 1 Eq, 3.6 mmol) in dry Toluene (10.0 mL) was added dropwise and the reaction mixture was stirred at room temperature for 18 h. The reaction mixture was quenched with sat. aq. NaHCO₃ (50 mL) and left to stir at rt for 1 h. The resulting suspension was extracted with EtOAc (3×50 mL), and the collected organic layers were dried over Na₂SO₄ and concentrated in vacuo. The crude product was purified by chromatography on silica gel (40 g cartridge, 25-100% EtOAc/iso-hexane) to afford sub-title compound (836 mg, 1.9 mmol, 52%).

m/z 396.9 (M+H)⁺ (ES+); 395.5 (M−H)⁻ (ES−).

¹H NMR (500 MHz, DMSO) δ 11.95 (s, 1H), 8.39 (d, J=10.7 Hz, 1H), 7.85 (dd, J=15.3, 8.7 Hz, 1H), 7.42-7.29 (m, 3H), 7.18-7.01 (m, 3H), 5.26-5.14 (m, 1H), 5.12-5.01 (m, 1H), 4.94 (q, J=13.2 Hz, 1H), 3.86 (d, J=3.8 Hz, 3H), 3.56-3.45 (m, 2H), 2.42-2.25 (m, 1H), 1.93-1.81 (m, 3H).

¹⁹F NMR (471 MHz, DMSO) δ −138.33 (d, J=25.0 Hz).

### Step 3: (R)-6-Fluoro-5-methoxy-3-prolyl-1H-indole

A stirred solution of the product from step 2 above (830 mg, 1 Eq, 1.88 mmol) and Pd/C (87 L) (240 mg, 5% Wt, 0.0598 Eq, 113 μmol) in EtOH (18.0 mL) under a nitrogen atmosphere at rt was charged with hydrogen (1 bar). The reaction mixture was stirred at rt for 2 h. The reaction mixture was filtered through glass microfibres. The solvent was removed in vacuo. The residue was dissolved in MeOH (50 mL) and Pd/C (87 L) (240 mg, 5% Wt, 0.0598 Eq, 113 μmol) was added. The mixture was subjected to a hydrogen atmosphere at 2 bar and 22° C. for 5 h. The reaction mixture was filtered through glass microfibres, rinsing with MeOH (5 mL). The solvent was removed in vacuo to afford sub-title compound (485 mg, 1.8 mmol, 93%).

m/z 263.2 (M+H)⁺ (ES+); 261.0 (M−H)⁻ (ES−)

¹H NMR (500 MHz, DMSO) δ 11.94 (s, 1H), 8.33 (d, J=1.4 Hz, 1H), 7.84 (d, J=8.7 Hz, 1H), 7.33 (d, J=11.4 Hz, 1H), 4.43-4.34 (m, 1H), 3.86 (s, 3H), 3.09-3.02 (m, 1H), 2.79-2.76 (m, 1H), 2.19-2.14 (m, 1H), 1.76-1.69 (m, 1H), 1.69-1.57 (m, 2H).

¹⁹F NMR (471 MHz, DMSO) δ −138.44 (d, J=5.7 Hz).

### Step 4: (R)-6-Fluoro-5-methoxy-3-(pyrrolidin-2-ylmethyl)-1H-indole

To a stirred solution of the product from step 4 above (485 mg, 1 Eq, 1.85 mmol) in dry Tetrahydrofuran (13.00 mL) under a nitrogen atmosphere at 0° C. was added LiAlH4 in THF (281 mg, 3.70 mL, 2.00 molar, 4 Eq, 7.40 mmol). The reaction mixture was heated to 66° C. and stirred for 2 h. The reaction mixture was cooled to 0° C. and quenched with 3:1 THF/water (16 mL). The mixture was warmed to rt and stirred for 1 h before Na₂SO₄ was added and the mixture was stirred for 18 h. The suspension was filtered, and the solvent was removed in vacuo to afford sub-title compound (457 mg, 1.6 mmol, 85%) as a brown gum.

m/z 249.2 (M+H)⁺ (ES+)

### Step 5: (R)-1-(2-((6-fluoro-5-methoxy-1H-indol-3-yl)methyl)pyrrolidin-1-yl)ethan-1-one

To a stirred solution of product from step 4 above (75.0 mg, 1 Eq, 257 μmol) in dry DCM (2.00 mL) under a nitrogen atmosphere at rt was added DIPEA (134 μL, 3 Eq, 770 μmol) and acetyl chloride (27.4 μL, 1.5 Eq, 385 μmol). The reaction mixture was stirred for 1 h at rt. The reaction mixture was concentrated in vacuo. The reaction mixture was partitioned between distilled water (10 mL) and DCM (10 mL). The aqueous layer was extracted with DCM (3×10 mL). The combined organic layers were collected, filtered through a phase separator, then concentrated in vacuo. The crude product was purified by chromatography on silica gel (12 g cartridge, 0-100% (2% NH₄OH in 3:1 EtOH/EtOAc) in iso-hexane) to afford sub-title compound (44 mg, 0.14 mmol, 53%).

m/z 291.2 (M+H)⁺ (ES+); 289.4 (M−H)⁻ (ES−)

### Step 6: (R)-3-((1-ethylpyrrolidin-2-yl)methyl)-6-fluoro-5-methoxy-1H-indole, Formic Acid

To a stirred solution of product from step 5 above (44.0 mg, 1 Eq, 136 μmol) in 2-MeTHF (1.00 mL) under a nitrogen atmosphere at rt was added LiAlH₄ in THF (170 μL, 2.4 molar, 3 Eq, 409 μmol). The reaction mixture was stirred at rt for 1.5 h. The reaction mixture was cooled to 0° C. and quenched by slow addition of THF/H2O (3:1, 4 mL). The mixture was stirred for 30 min at rt. Na₂SO₄ (ca 3 g) and EtOAc (15 mL) were added to the reaction mixture and stirred vigorously for 5 min. The suspension was filtered, and the filtrate was concentrated in vacuo. The crude material was dissolved in 1.3 mL with DMSO, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters X-Select CSH C18 ODB prep column, 130 Å, 5 μm, 30 mm×100 mm, flow rate 40 mL min-1 eluting with a 0.1% Formic acid in water-MeCN gradient over 17.5 mins using UV across all wavelengths with PDA as well as a QDA and ELS detector. At-column dilution pump gives 2 mL min-1 Methanol over the entire method, which is included in the following MeCN percentages. Gradient information: 0.0-0.5 min, 10% MeCN; 0.5-15.5 min, ramped from 10% MeCN to 20% MeCN; 15.5-15.6 min, ramped from 20% MeCN to 100% MeCN; 15.6-17.5 min, held at 100% MeCN. The clean fractions were evaporated in a freeze-dryer to afford title compound (19.7 mg, 57 mol, 42%) as a pale yellow gum.

m/z 277.3 (M+H)⁺ (ES+)

¹H NMR (500 MHz, DMSO) δ 10.70 (s, 1H), 8.25 (s, 1H), 7.15 (d, J=3.7 Hz, 1H), 7.13 (d, J=7.1 Hz, 1H), 7.09 (d, J=2.4 Hz, 1H), 3.83 (s, 3H), 3.20-3.13 (m, 1H), 3.10-2.97 (m, 2H), 2.73 (s, 1H), 2.56 (dd, J=14.2, 9.1 Hz, 1H), 2.32 (dt, J=13.7, 7.0 Hz, 1H), 2.23 (q, J=8.7 Hz, 1H), 1.79-1.69 (m, 1H), 1.68-1.60 (m, 2H), 1.54-1.45 (m, 1H), 1.10 (t, J=7.2 Hz, 3H). One exchangeable proton not observed—Formic acid salt.

The product (DMSO solution, 40 μl) was diluted with MeOH (1 mL) and screened by SFC (Waters UPC2, with a PDA and a QDA detector). The optimal conditions are using a ChiralpaK IH, 4.6×250 mm, 5 μm, with mobile phase 20% MeOH (28 mM NH3), 80% CO2. The flow rate was 4 mL/min. The run time was 3 min.

ee=55.40%.

### Example 9: Synthesis of (R)-3-((2-((6-fluoro-5-methoxy-1H-indol-3-yl)methyl)pyrrolidin-1-yl)methyl)isoxazole, Formic Acid (Compound 2B)

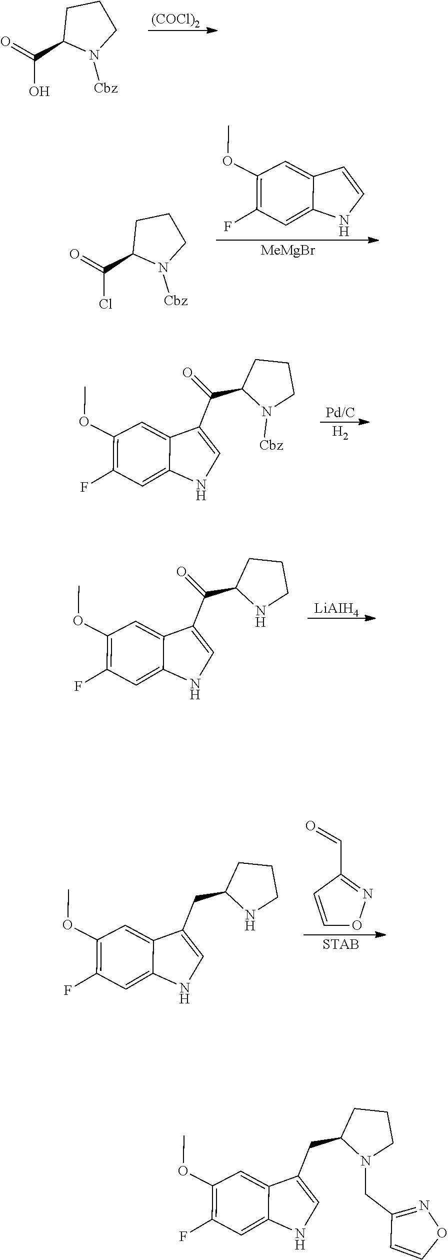

### Step 1: Benzyl (R)-2-(chlorocarbonyl)pyrrolidine-1-carboxylate

To a stirred solution of ((benzyloxy)carbonyl)-D-proline (1.32 g, 1 Eq, 5.30 mmol) in DCM (15.0 mL) under a nitrogen atmosphere at 0° C. was added oxalyl dichloride (3.33 g, 2.30 mL, 4.96 Eq, 26.3 mmol). The reaction mixture was stirred at rt for 3 h. The reaction mixture was concentrated in vacuo, and the resulting crude was suspended in iso-hexane (3×15 mL) and dried in vacuo to provide the sub-title compound (1.42 g, 5.30 mmol, 100%) as a pale-yellow oil.

m/z 264.2 (M−Cl+MeOH)⁺ (ES⁺)

### Step 2: Benzyl (R)-2-(6-fluoro-5-methoxy-1H-indole-3-carbonyl)pyrrolidine-1-carboxylate

To a stirred solution of 6-fluoro-5-methoxy-1H-indole (1.75 g, 2 Eq, 10.6 mmol) in dry Toluene (10.0 mL) under a nitrogen atmosphere at rt was added MeMgBr in diethyl ether (1.26 g, 3.54 mL, 3.00 molar, 2 Eq, 10.6 mmol) dropwise over 2 min. THF (4 mL) was added. The reaction mixture was stirred at rt for 4 h. A solution of product from step 1 above (1.42 g, 1 Eq, 5.30 mmol) in dry Toluene (15.0 mL) was added dropwise and the reaction mixture was stirred at rt for 72 h. The reaction mixture was quenched with sat. aq. NaHCO₃ (50 mL) and left to stir at rt for 1 h. The resulting suspension was extracted with EtOAc (3×50 mL), and the collected organic layers were dried over Na₂SO₄ and concentrated in vacuo. The crude product was purified by chromatography on silica gel (80 g cartridge, 25-100% EtOAc/iso-hexane) to afford sub-title compound (1.32 g, 3.2 mmol, 60%, 95% Purity).

m/z 397.1 (M+H)⁺ (ES+); 395.6 (M−H)⁻ (ES−)

¹H NMR (500 MHz, DMSO) δ 11.95 (s, 1H), 8.39 (d, J=10.6 Hz, 1H), 7.89-7.80 (m, 1H), 7.42-7.31 (m, 3H), 7.17-7.00 (m, 3H), 5.24-5.12 (m, 1H), 5.10-5.00 (m, 1H), 4.94 (q, J=13.2 Hz, 1H), 3.86 (s, 3H), 3.56-3.47 (m, 2H), 2.42-2.28 (m, 2H), 1.91-1.80 (m, 2H).

¹⁹F NMR (471 MHz, DMSO) δ −138.33 (m).

### Step 3: (R)-6-Fluoro-5-methoxy-3-prolyl-1H-indole

A stirred solution of the product from step 2 above (1.32 g, 1 Eq, 3.33 mmol) and Pd/C (87 L) (354 mg, 5% Wt, 0.05 Eq, 166 μmol) in MeOH (100.0 mL) under a nitrogen atmosphere at rt was charged with hydrogen (1 bar). The reaction mixture was stirred at rt for 18 h. The reaction mixture was filtered through glass microfibres, rinsing with MeOH (5 mL). The solvent was removed in vacuo to afford sub-title compound (840 mg, 3.0 mmol, 91%).

m/z 263.5 (M+H)⁺ (ES+); 261.0 (M−H)⁻ (ES−)

¹H NMR (500 MHz, DMSO) δ 8.34 (s, 1H), 7.84 (d, J=8.7 Hz, 1H), 7.33 (d, J=11.4 Hz, 1H), 4.40 (dd, J=8.7, 6.0 Hz, 1H), 3.86 (s, 3H), 3.06 (dt, J=11.6, 6.2 Hz, 1H), 2.78 (dt, J=10.4, 6.5 Hz, 1H), 2.22-2.11 (m, 1H), 1.77-1.69 (m, 1H), 1.69-1.59 (m, 2H). Two exchangeable protons not observed.

¹⁹F NMR (471 MHz, DMSO) δ −138.43.

### Step 4: (R)-6-Fluoro-5-methoxy-3-(pyrrolidin-2-ylmethyl)-1H-indole

To a stirred suspension of the product from step 3 above (835 mg, 1 Eq, 3.18 mmol) in dry THF (30.0 mL) under a nitrogen atmosphere at 0° C. was added LiAlH₄ in THF (6.50 mL, 2.00 molar, 4.08 Eq, 13.0 mmol). The reaction mixture was heated to 66° C. and stirred for 2 h. The reaction mixture was cooled to 0° C. and quenched with 3:1 THF/water (16 mL). The mixture was warmed to rt and stirred for 1 h before being filtered. Na₂SO₄ was added to the filtrate and the mixture was filtered and concentrated in vacuo to afford sub-title compound (750 mg, 1.9 mmol, 61%) as a brown gum.

m/z 249.5 (M+H)⁺ (ES+)

### Step 5: (R)-3-((2-((6-fluoro-5-methoxy-1H-indol-3-yl)methyl)pyrrolidin-1-yl)methyl)isoxazole, Formic Acid

To a stirred solution of the product from step 4 above (100 mg, 64% Wt, 1 Eq, 258 μmol) in dry THF (2.00 mL) at 22° C. was added isoxazole-3-carbaldehyde (37.5 mg, 1.5 Eq, 387 μmol) and sodium triacetoxyhydroborate (164 mg, 3 Eq, 773 μmol). The reaction mixture left to stir for 20 h. The reaction mixture was quenched with sat. aq. sodium bicarbonate solution (5 mL) and left to stir for 1 hour. The aq. suspension was extracted with EtOAc (3×15 mL) and the collected organic layers dried (Na₂SO₄), filtered and concentrated in vacuo. The crude was dissolved in 1.8 mL with DMSO, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters X-Select CSH C18 ODB prep column, 130 Å, 5 μm, 30 mm×100 mm, flow rate 40 mL min⁻¹ eluting with a 0.1% Formic acid in water-MeCN gradient over 17.5 mins using UV across all wavelengths with PDA as well as a QDA and ELS detector. At-column dilution pump gives 2 mL min⁻¹ Methanol over the entire method, which is included in the following MeCN percentages. Gradient information: 0.0-0.5 min, 10% MeCN; 0.5-15.5 min, ramped from 10% MeCN to 22.5% MeCN; 15.5-15.6 min, ramped from 22.5% MeCN to 100% MeCN; 15.6-17.5 min, held at 100% MeCN. The clean fractions were evaporated to obtain the title compound (30.1 mg, 78 mol, 30%) as an orange gum.

m/z 330.199 (M+H)⁺ (ES+)

¹H NMR (500 MHz, DMSO) δ 10.68-10.65 (m, 1H), 8.86 (d, J=1.6 Hz, 1H), 8.15 (s, 1H), 7.20-7.07 (m, 3H), 6.54 (d, J=1.6 Hz, 1H), 4.07 (d, J=14.0 Hz, 1H), 3.85 (s, 3H), 3.68 (d, J=14.0 Hz, 1H), 3.01 (dd, J=14.0, 3.7 Hz, 1H), 2.90 (ddd, J=9.8, 7.1, 2.8 Hz, 1H), 2.75 (ddt, J=11.2, 7.3, 3.7 Hz, 1H), 2.31 (td, J=8.9, 7.5 Hz, 1H), 1.73-1.43 (m, 3H). +1 exchangeable proton not observed. +2 protons underneath water peak.

¹⁹F NMR (471 MHz, DMSO) δ −141.45.

### Example 10: Synthesis of (R)-6-fluoro-5-methoxy-3-((1-((1-methyl-1H-1,2,4-triazol-3-yl)methyl)pyrrolidin-2-yl)methyl)-1H-indole, Formic Acid (Compound 27B)

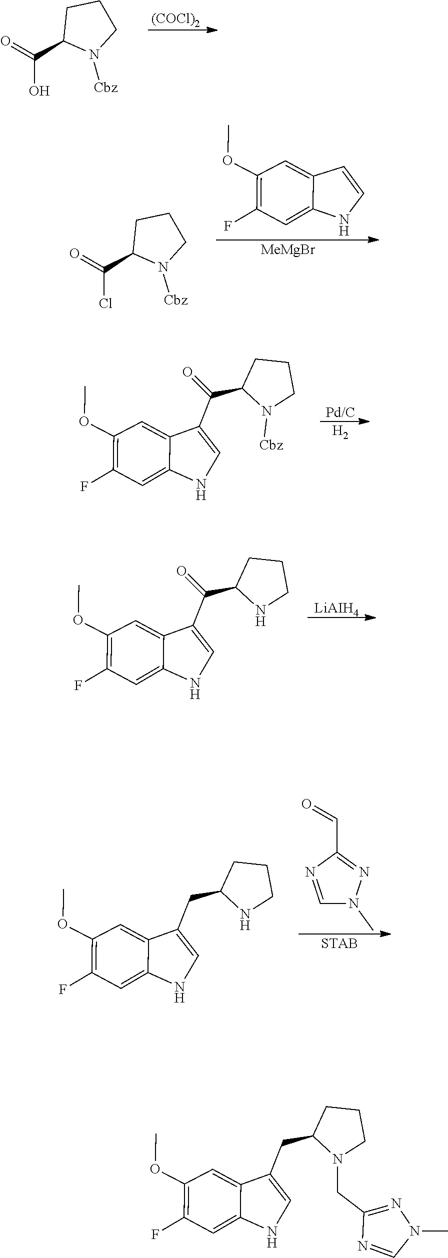

### Step 1: Benzyl (R)-2-(chlorocarbonyl)pyrrolidine-1-carboxylate

To a stirred solution of ((benzyloxy)carbonyl)-D-proline (1.32 g, 1 Eq, 5.30 mmol) in DCM (15.0 mL) under a nitrogen atmosphere at 0° C. was added oxalyl dichloride (3.33 g, 2.30 mL, 4.96 Eq, 26.3 mmol). The reaction mixture was stirred at rt for 3 h. The reaction mixture was concentrated in vacuo, and the resulting crude was suspended in iso-hexane (3×15 mL) and dried in vacuo to provide the sub-title compound (1.42 g, 5.30 mmol, 100%) as a pale-yellow oil.

m/z 264.2 (M−Cl+MeOH)⁺ (ES⁺)

### Step 2: Benzyl (R)-2-(6-fluoro-5-methoxy-1H-indole-3-carbonyl)pyrrolidine-1-carboxylate

To a stirred solution of 6-fluoro-5-methoxy-1H-indole (1.75 g, 2 Eq, 10.6 mmol) in dry Toluene (10.0 mL) under a nitrogen atmosphere at rt was added MeMgBr in diethyl ether (1.26 g, 3.54 mL, 3.00 molar, 2 Eq, 10.6 mmol) dropwise over 2 min. THF (4 mL) was added. The reaction mixture was stirred at rt for 4 h. A solution of product from step 1 above (1.42 g, 1 Eq, 5.30 mmol) in dry Toluene (15.0 mL) was added dropwise and the reaction mixture was stirred at rt for 72 h. The reaction mixture was quenched with sat. aq. NaHCO₃ (50 mL) and left to stir at rt for 1 h. The resulting suspension was extracted with EtOAc (3×50 mL), and the collected organic layers were dried over Na₂SO₄ and concentrated in vacuo. The crude product was purified by chromatography on silica gel (80 g cartridge, 25-100% EtOAc/iso-hexane) to afford sub-title compound (1.32 g, 3.2 mmol, 60%, 95% Purity).

m/z 397.1 (M+H)⁺ (ES+); 395.6 (M−H)⁻ (ES−)

¹H NMR (500 MHz, DMSO) δ 11.95 (s, 1H), 8.39 (d, J=10.6 Hz, 1H), 7.89-7.80 (m, 1H), 7.42-7.31 (m, 3H), 7.17-7.00 (m, 3H), 5.24-5.12 (m, 1H), 5.10-5.00 (m, 1H), 4.94 (q, J=13.2 Hz, 1H), 3.86 (s, 3H), 3.56-3.47 (m, 2H), 2.42-2.28 (m, 2H), 1.91-1.80 (m, 2H).

¹⁹F NMR (471 MHz, DMSO) δ −138.33 (m).

### Step 3: (R)-6-Fluoro-5-methoxy-3-prolyl-1H-indole

A stirred solution of the product from step 2 above (1.32 g, 1 Eq, 3.33 mmol) and Pd/C (87 L) (354 mg, 5% Wt, 0.05 Eq, 166 μmol) in MeOH (100.0 mL) under a nitrogen atmosphere at rt was charged with hydrogen (1 bar). The reaction mixture was stirred at rt for 18 h. The reaction mixture was filtered through glass microfibres, rinsing with MeOH (5 mL). The solvent was removed in vacuo to afford sub-title compound (840 mg, 3.0 mmol, 91%).

m/z 263.5 (M+H)⁺ (ES+); 261.0 (M−H)⁻ (ES−)

¹H NMR (500 MHz, DMSO) δ 8.34 (s, 1H), 7.84 (d, J=8.7 Hz, 1H), 7.33 (d, J=11.4 Hz, 1H), 4.40 (dd, J=8.7, 6.0 Hz, 1H), 3.86 (s, 3H), 3.06 (dt, J=11.6, 6.2 Hz, 1H), 2.78 (dt, J=10.4, 6.5 Hz, 1H), 2.22-2.11 (m, 1H), 1.77-1.69 (m, 1H), 1.69-1.59 (m, 2H). Two exchangeable protons not observed.

¹⁹F NMR (471 MHz, DMSO) δ −138.43.

### Step 4: (R)-6-Fluoro-5-methoxy-3-(pyrrolidin-2-ylmethyl)-1H-indole

To a stirred suspension of the product from step 3 above (835 mg, 1 Eq, 3.18 mmol) in dry THF (30.0 mL) under a nitrogen atmosphere at 0° C. was added LiAlH₄ in THF (6.50 mL, 2.00 molar, 4.08 Eq, 13.0 mmol). The reaction mixture was heated to 66° C. and stirred for 2 h. The reaction mixture was cooled to 0° C. and quenched with 3:1 THF/water (16 mL). The mixture was warmed to rt and stirred for 1 h before being filtered. Na₂SO₄ was added to the filtrate and the mixture was filtered and concentrated in vacuo to afford sub-title compound (750 mg, 1.9 mmol, 61%) as a brown gum.

m/z 249.5 (M+H)⁺ (ES+)

### Step 5: (R)-6-fluoro-5-methoxy-3-((1-((1-methyl-1H-1,2,4-triazol-3-yl)methyl)pyrrolidin-2-yl)methyl)-1H-indole, Formic Acid

To a stirred solution of the product from step 4 above (100 mg, 64% Wt, 1 Eq, 258 μmol) in dry THF (2.00 mL) at 22° C. was added 1-methyl-1H-1,2,4-triazole-3-carbaldehyde (43.0 mg, 1.5 Eq, 387 μmol) and sodium triacetoxyhydroborate (164 mg, 3 Eq, 773 μmol). The reaction mixture left to stir for 20 h. The reaction mixture was quenched with sat. aq. sodium bicarbonate solution (5 mL) and left to stir for 1 hour. The aq. suspension was extracted with EtOAc (3×15 mL) and the collected organic layers dried (Na₂SO₄), filtered and concentrated in vacuo. The crude was dissolved in 1.9 mL with DMSO, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters X-Select CSH C18 ODB prep column, 130 Å, 5 μm, 30 mm×100 mm, flow rate 40 mL min⁻¹ eluting with a 0.1% Formic acid in water-MeCN gradient over 17.5 mins using UV across all wavelengths with PDA as well as a QDA and ELS detector. At-column dilution pump gives 2 mL min⁻¹ Methanol over the entire method, which is included in the following MeCN percentages. Gradient information: 0.0-0.5 min, 10% MeCN; 0.5-15.5 min, ramped from 10% MeCN to 20% MeCN; 15.5-15.6 min, ramped from 20% MeCN to 100% MeCN; 15.6-17.5 min, held at 100% MeCN. The clean fractions were evaporated to obtain the title compound (27.7 mg, 70 mol, 27%) as an orange gum.

m/z 343.896 (M+H)⁺ (ES+)

¹H NMR (500 MHz, DMSO) δ 10.65 (s, 1H), 8.38 (s, 1H), 8.18 (s, 1H), 7.29 (d, J=8.5 Hz, 1H), 7.14-7.07 (m, 2H), 4.02-3.95 (m, 1H), 3.85 (d, J=9.3 Hz, 6H), 3.70 (d, J=13.7 Hz, 1H), 3.06 (dd, J=14.0, 3.5 Hz, 1H), 2.97-2.90 (m, 1H), 2.83 (s, 1H), 2.42 (ddd, J=36.2, 14.8, 8.2 Hz, 2H), 1.64-1.37 (m, 3H). +1 exchangeable proton not observed+1 Proton under water peak.

¹⁹F NMR (471 MHz, DMSO) δ −141.49.

### Example 11: Synthesis of 6-fluoro-5-methoxy-3-(((2S,4R)-4-methoxy-1-methylpyrrolidin-2-yl)methyl)-1H-indole, Formic Acid (Compound 12D)

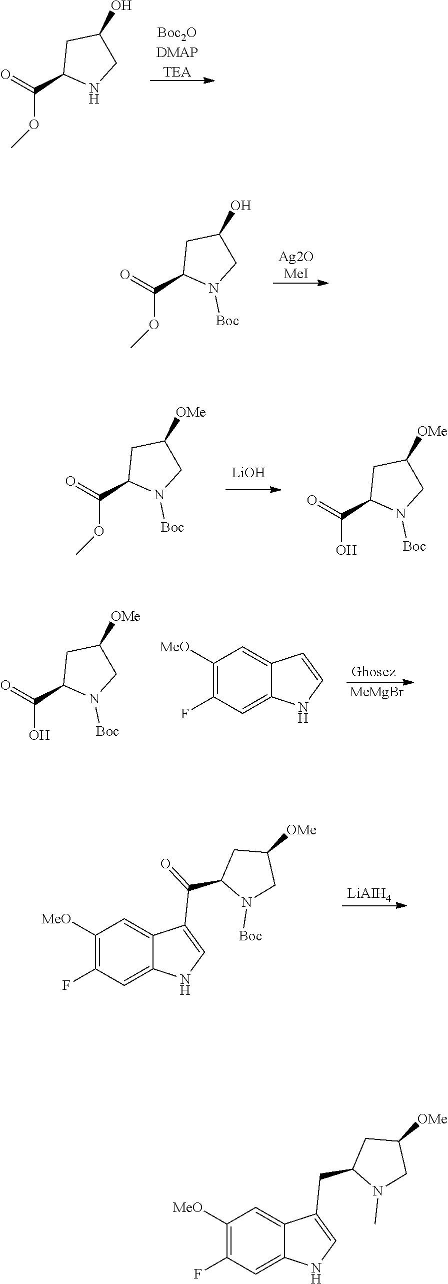

### Step 1: 1-(tert-butyl) 2-methyl (2R,4R)-4-hydroxypyrrolidine-1,2-dicarboxylate

To a stirred solution of methyl (2R,4R)-4-hydroxy-2-pyrrolidinecarboxylate hydrochloride (5.00 g, 1 Eq, 27.5 mmol) in dry DCM (50.0 mL) under a nitrogen atmosphere at rt was added triethylamine (8.71 g, 12.0 mL, 3.13 Eq, 86.1 mmol), DMAP (0.24 g, 0.071 Eq, 2.0 mmol) and boc₂o (6.31 g, 6.64 mL, 1.05 Eq, 28.9 mmol) portion wise. The reaction mixture was stirred over the weekend. The reaction mixture was diluted with Water (50 mL) and transferred into a separating funnel. The aq. layer was extracted with DCM (3×25 mL). The combined organic layers were collected, washed with brine (100 mL), dried (Na₂SO₄), filtered and concentrated in vacuo to afford the sub-title compound (6.787 g, 24 mmol, 85%).

¹H NMR (500 MHz, DMSO) δ 5.01 (s, 0.5H), 4.96 (s, 0.5H), 4.25-4.16 (m, 2H), 3.63 (s, 2H), 3.60 (s, 1H), 3.48 (ddd, J 10.5, 5.5, 3.9 Hz, 1H), 3.12 (td, J=11.0, 4.0 Hz, 1H), 2.38-2.24 (m, 1H), 1.84 (t, J=4.7 Hz, 0.5H), 1.81 (t, J=4.7 Hz, 0.5H), 1.39 (s, 4H), 1.33 (s, 5H).

### Step 2: 1-(tert-butyl) 2-methyl (2R,4R)-4-methoxypyrrolidine-1,2-dicarboxylate

To a stirred solution the product from step 1 above (1.95 g, 1 Eq, 7.95 mmol) in dry MeCN (39.8 mL) under a nitrogen atmosphere at rt was added silver oxide (7.37 g, 4 Eq, 31.8 mmol) and methyl iodide (11.3 g, 4.97 mL, 10 Eq, 79.5 mmol). The reaction mixture was stirred for 18 hours. The reaction mixture was filtered on Celite, washed with MeCN and the filtrate was concentrated in vacuo to afford the sub-title compound (1.68 g, 6.48 mmol, 81.5%) as a dark yellow oil.

m/z 160.111 (M−Boc+H)⁺ (ES+)

### Step 3: (2R,4R)-1-(tert-butoxycarbonyl)-4-methoxypyrrolidine-2-carboxylic acid

To a solution of the product from step 2 above (1.68 g, 1 Eq, 6.48 mmol) in THF (15.0 mL) was added LiOH (310 mg, 2 Eq, 13.0 mmol) and Water (1.50 mL). The reaction mixture was stirred at room temperature overnight. The mixture was concentrated in vacuo. The pH of the resulting residue was adjusted to 2 with HCl (1.0 M, ~15 mL) then the mixture was extracted with EtOAc (3×100 mL). The combined organic layers were washed with brine (250 mL), dried (MgSO₄), filtered and concentrated in vacuo to give the sub-title compound (1.08 g, 4.40 mmol, 68.0%) as a clear oil.

m/z 145.684 (M−Boc+H)⁺ (ES+)

m/z 244.390 (M−H)⁻ (ES−)

### Step 4: Tert-butyl (2R,4R)-2-(6-fluoro-5-methoxy-1H-indole-3-carbonyl)-4-methoxypyrrolidine-1-carboxylate

To a stirred solution of 6-fluoro-5-methoxy-1H-indole (606 mg, 2 Eq, 3.67 mmol) in dry Toluene (5.00 mL) and THF (5.00 mL) under a nitrogen atmosphere at 22° C. was added MeMgBr in Et₂O (438 mg, 1.22 mL, 3.00 molar, 2 Eq, 3.67 mmol) dropwise over 1 minute. In a separate vial, (the product from step 3 above (450 mg, 1 Eq, 1.83 mmol) dissolved in dry DCM (5.00 mL) under a nitrogen atmosphere was treated with 1-Chloro-NN,2-Trimethylpropenylamine (270 mg, 267 μL, 1.1 Eq, 2.02 mmol, Ghosez reagent). After 30 minutes, the acid chloride solution was added dropwise to the stirring solution of the deprotonated indole. The reaction mixture was stirred for 3 hours. The reaction mixture was quenched with sat. aq. sodium bicarbonate (50 mL) and left to stir at room temperature for 1 hour. The resulting suspension was extracted with EtOAc (3×50 mL), and the collected organic layers were dried (Na₂SO₄), filtered and concentrated in vacuo. The crude product was purified by chromatography on silica gel (80 g cartridge, 25-100% EtOAc/isohexane) to afford the sub-title compound (238 mg, 0.58 mmol, 31%).

m/z 292.918 (M−Boc+H)⁺ (ES+)

¹H NMR (500 MHz, DMSO) δ 11.90 (d, J=11.1 Hz, 1H), 8.32-8.27 (m, 1H), 7.83 (t, J=9.4 Hz, 1H), 7.33 (dd, J=11.3, 2.1 Hz, 1H), 5.08-4.96 (m, 1H), 4.05-3.94 (m, 1H), 3.86 (d, J=3.9 Hz, 3H), 3.78 (dt, J=10.8, 6.8 Hz, 1H), 3.27-3.10 (m, 4H), 2.65 (dq, J=15.5, 8.1 Hz, 1H), 1.76 (dt, J=13.0, 6.5 Hz, 1H), 1.40 (s, 4H), 1.13 (s, 5H).

¹⁹F NMR (471 MHz, DMSO) δ −138.41 (s).

### Step 5: 6-fluoro-5-methoxy-3-(((2S,4R)-4-methoxy-1-methylpyrrolidin-2-yl)methyl)-1H-indole, Formic Acid

To a stirred solution the product from step 4 above (50.0 mg, 1 Eq, 127 μmol) in dry THF (3.00 mL) under a nitrogen atmosphere at 22° C. was added LiAlH₄ (29.0 mg, 382 μL, 2.00 molar, 6 Eq, 764 μmol). The reaction mixture was heated to 66° C. and stirred for 2 hours. The mixture was cooled to 0° C. and quenched with 3:1 THF/water mixture (2.5 mL). The mixture was warmed to rt and stirred for 1 h before being filtered. Na₂SO₄ was added to the filtrate and the mixture was filtered and concentrated in vacuo. The crude was dissolved in 1.3 mL with DMSO, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge BEH C18 ODB prep column, 130 Å, 5 μm, 30 mm×100 mm, flow rate 40 mL min⁻¹ eluting with a 0.3% formic acid in water-MeCN gradient over 17.5 mins using UV across all wavelengths with PDA as well as a QDA and ELS detector. At-column dilution pump gives 2 mL min⁻¹ Methanol over the entire method, which is included in the following MeCN percentages. Gradient information: 0.0-0.5 min, 15% MeCN; 0.5-15.5 min, ramped from 15% MeCN to 45% MeCN; 15.5-15.6 min, ramped from 45% MeCN to 100% MeCN; 15.6-17.5 min, held at 100% MeCN. The clean fractions were evaporated in a Freeze Dry in order to obtain the title compound (5.13 mg, 15 mol, 12%) as a colourless gum.

m/z 293.187 (M+H)⁺ (ES+)

¹H NMR (500 MHz, DMSO) δ 10.70 (s, 1H), 8.17 (s, 1H), 7.17-7.08 (m, 3H), 3.83 (s, 3H), 3.72 (dt, J=10.2, 7.2 Hz, 1H), 3.12 (s, 3H), 3.09-3.00 (m, 2H), 2.58-2.51 (m, 1H), 2.41-2.35 (m, 1H), 2.32 (s, 3H), 2.22 (dd, J=10.7, 6.0 Hz, 1H), 2.07 (dt, J=13.3, 7.3 Hz, 1H), 1.44 (ddd, J=13.0, 8.9, 3.8 Hz, 1H). +1 exchangeable proton not observed.

¹⁹F NMR (471 MHz, DMSO) δ −141.40.

### Example 12: Synthesis of (R)-6-fluoro-5-methoxy-3-((1-(oxetan-3-yl)pyrrolidin-2-yl)methyl)-1H-indole (Compound 1B)

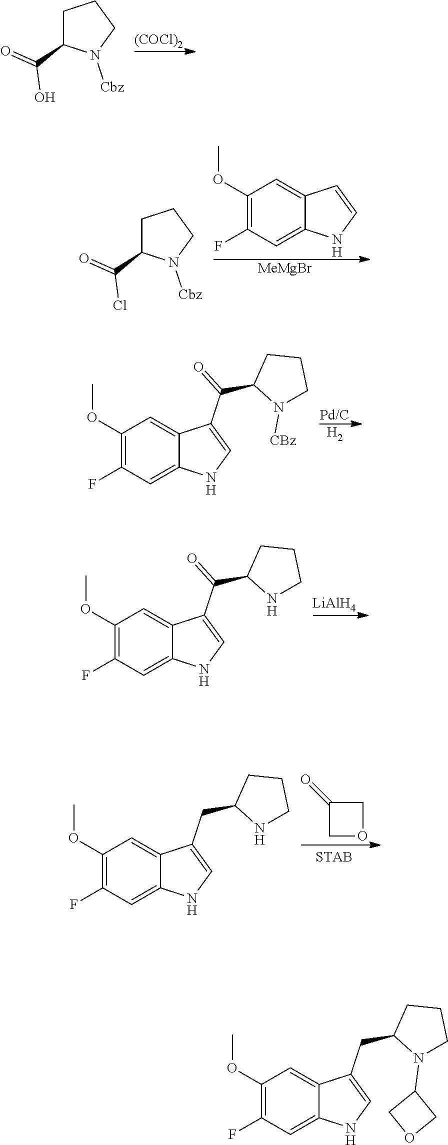

### Step 1: Benzyl (R)-2-(chlorocarbonyl)pyrrolidine-1-carboxylate

To a stirred solution of ((benzyloxy)carbonyl)-D-proline (0.90 g, 1 Eq, 3.6 mmol) in DCM (10.0 mL) under a nitrogen atmosphere at 0° C. was added oxalyl dichloride (2.3 g, 1.6 mL, 5 Eq, 18 mmol). The reaction mixture was stirred at rt for 3 h. The reaction mixture was concentrated in vacuo, and the resulting crude was suspended in iso-hexane (3×20 mL) and dried in vacuo to provide the sub-title compound (0.97 g, 3.6 mmol, 100%) as a yellow oil.

m/z 220.6 (M−CO₂Cl+MeOH)⁺ (ES⁺)

### Step 2: Benzyl (R)-2-(6-fluoro-5-methoxy-1H-indole-3-carbonyl)pyrrolidine-1-carboxylate

To a stirred solution of 6-fluoro-5-methoxy-1H-indole (1.05 g, 1.8 Eq, 6.36 mmol) in dry Toluene (10.0 mL) under a nitrogen atmosphere at rt was added MeMgBr in diethyl ether (0.82 g, 2.3 mL, 3.00 molar, 1.9 Eq, 6.9 mmol) dropwise over 2 min. THF (4 mL) was added. The reaction mixture was stirred at rt for 4 h. A solution of product from step 1 above (0.97 g, 1 Eq, 3.6 mmol) in dry Toluene (10.0 mL) was added dropwise and the reaction mixture was stirred at room temperature for 18 h. The reaction mixture was quenched with sat. aq. NaHCO₃ (50 mL) and left to stir at rt for 1 h. The resulting suspension was extracted with EtOAc (3×50 mL), and the collected organic layers were dried over Na₂SO₄ and concentrated in vacuo. The crude product was purified by chromatography on silica gel (40 g cartridge, 25-100% EtOAc/iso-hexane) to afford sub-title compound (836 mg, 1.9 mmol, 52%).

m/z 396.9 (M+H)⁺ (ES+); 395.5 (M−H)⁻ (ES−).

¹H NMR (500 MHz, DMSO) δ 11.95 (s, 1H), 8.39 (d, J=10.7 Hz, 1H), 7.85 (dd, J=15.3, 8.7 Hz, 1H), 7.42-7.29 (m, 3H), 7.18-7.01 (m, 3H), 5.26-5.14 (m, 1H), 5.12-5.01 (m, 1H), 4.94 (q, J=13.2 Hz, 1H), 3.86 (d, J=3.8 Hz, 3H), 3.56-3.45 (m, 2H), 2.42-2.25 (m, 1H), 1.93-1.81 (m, 3H).

¹⁹F NMR (471 MHz, DMSO) δ −138.33 (d, J=25.0 Hz).

### Step 3: (R)-6-Fluoro-5-methoxy-3-prolyl-1H-indole

A stirred solution of the product from step 2 above (830 mg, 1 Eq, 1.88 mmol) and Pd/C (87 L) (240 mg, 5% Wt, 0.0598 Eq, 113 μmol) in EtOH (18.0 mL) under a nitrogen atmosphere at rt was charged with hydrogen (1 bar). The reaction mixture was stirred at rt for 2 h. The reaction mixture was filtered through glass microfibres. The solvent was removed in vacuo. The residue was dissolved in MeOH (50 mL) and Pd/C (87 L) (240 mg, 5% Wt, 0.0598 Eq, 113 μmol) was added. The mixture was subjected to a hydrogen atmosphere at 2 bar and 22° C. for 5 h. The reaction mixture was filtered through glass microfibres, rinsing with MeOH (5 mL). The solvent was removed in vacuo to afford sub-title compound (485 mg, 1.8 mmol, 93%).

m/z 263.2 (M+H)⁺ (ES+); 261.0 (M−H)⁻ (ES−)

¹H NMR (500 MHz, DMSO) δ 11.94 (s, 1H), 8.33 (d, J=1.4 Hz, 1H), 7.84 (d, J=8.7 Hz, 1H), 7.33 (d, J=11.4 Hz, 1H), 4.43-4.34 (m, 1H), 3.86 (s, 3H), 3.09-3.02 (m, 1H), 2.79-2.76 (m, 1H), 2.19-2.14 (m, 1H), 1.76-1.69 (m, 1H), 1.69-1.57 (m, 2H).

¹⁹F NMR (471 MHz, DMSO) δ −138.44 (d, J=5.7 Hz).

### Step 4: (R)-6-Fluoro-5-methoxy-3-(pyrrolidin-2-ylmethyl)-1H-indole

To a stirred solution of the product from step 3 above (485 mg, 1 Eq, 1.85 mmol) in dry Tetrahydrofuran (13.00 mL) under a nitrogen atmosphere at 0° C. was added LiAlH4 in THF (281 mg, 3.70 mL, 2.00 molar, 4 Eq, 7.40 mmol). The reaction mixture was heated to 66° C. and stirred for 2 h. The reaction mixture was cooled to 0° C. and quenched with 3:1 THF/water (16 mL). The mixture was warmed to rt and stirred for 1 h before Na₂SO₄ was added and the mixture was stirred for 18 h. The suspension was filtered, and the solvent was removed in vacuo to afford sub-title compound (457 mg, 1.6 mmol, 85%) as a brown gum.

m/z 249.2 (M+H)⁺ (ES+)

### Step 5: (R)-6-fluoro-5-methoxy-3-((1-(oxetan-3-yl)pyrrolidin-2-yl)methyl)-1H-indole

To a stirred solution of product from step 4 above (75.0 mg, 1 Eq, 211 μmol) in dry THF (2.00 mL) at 22° C. was added Oxentan-3-on (20.3 μL, 1.5 Eq, 317 μmol). The reaction mixture was stirred for 1 h. Sodium triacetoxyhydroborate (134 mg, 3 Eq, 634 μmol) was added and the reaction mixture left to stir for 20 h. The reaction mixture was quenched with sat. aq NaHCO₃ (5 mL) and left to stir for 1 h. The aqueous suspension was extracted into EtOAc (3×15 mL) and the combined organic layers dried over Na₂SO₄, filtered and concentrated in vacuo. The crude product was purified by chromatography on silica gel (12 g cartridge, 0-50% (2% NH₄OH in 3:1 EtOH/EtOAc) in iso-hexane) to afford title compound (24.3 mg, 75 mol, 35%) as a dark orange gum.

m/z 305.2 (M+H)⁺ (ES+)

¹H NMR (500 MHz, DMSO) δ 10.68 (s, 1H), 7.13 (d, J=11.8 Hz, 1H), 7.11 (d, J=8.4 Hz, 1H), 7.08 (d, J=2.3 Hz, 1H), 4.64-4.59 (m, 2H), 4.59 (t, J=6.3 Hz, 1H), 4.53 (t, 1H), 3.99 (p, J=6.9 Hz, 1H), 3.83 (s, 3H), 2.96 (m, 1H), 2.77-2.69 (m, 2H), 2.49-2.44 (m, 1H), 2.41-2.34 (m, 1H), 1.73-1.56 (m, 3H), 1.52-1.43 (m, 1H).

¹⁹F NMR (471 MHz, DMSO) δ −141.42.

The product was dissolved in MeOH to 1 mg/mL and screened by SFC (Waters UPC2, with a PDA and a QDA detector). The optimal conditions are using a Lux iC5, 4.6×250 mm, 5 μm, with mobile phase 35% MeOH (28 mM NH₃), 65% CO2. The flow rate was 4 mL/min. The run time was 5 min.

ee=49.96%

### Example 13: Synthesis of 6-fluoro-5-methoxy-3-(((2S,4R)-4-methoxypyrrolidin-2-yl)methyl)-1H-indole (Compound 13C)

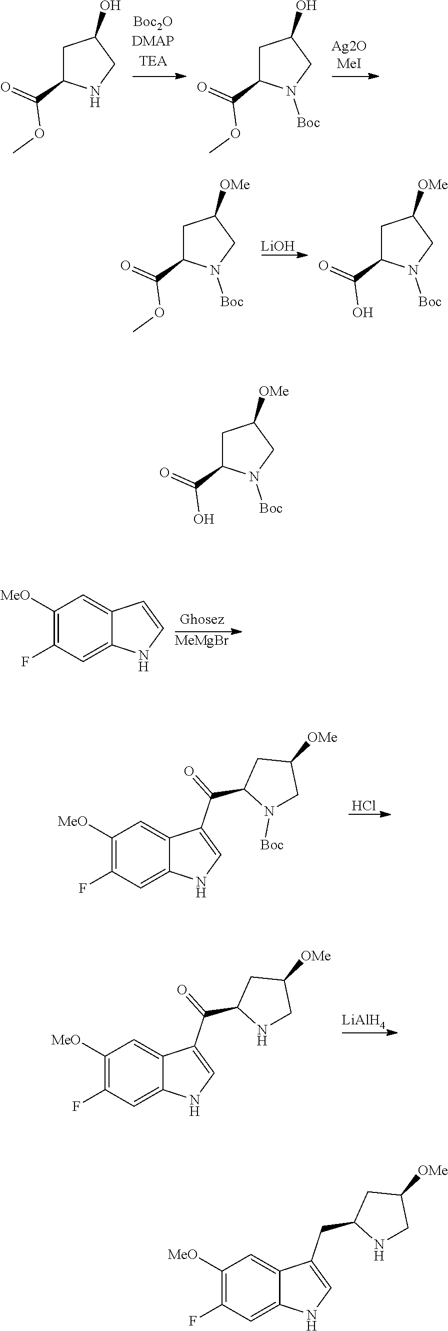

### Step 1: 1-(tert-butyl) 2-methyl (2R,4R)-4-hydroxypyrrolidine-1,2-dicarboxylate

To a stirred solution of methyl (2R,4R)-4-hydroxy-2-pyrrolidinecarboxylate hydrochloride (5.00 g, 1 Eq, 27.5 mmol) in dry DCM (50.0 mL) under a nitrogen atmosphere at rt was added triethylamine (8.71 g, 12.0 mL, 3.13 Eq, 86.1 mmol), DMAP (0.24 g, 0.071 Eq, 2.0 mmol) and boc₂o (6.31 g, 6.64 mL, 1.05 Eq, 28.9 mmol) portion wise. The reaction mixture was stirred over the weekend. The reaction mixture was diluted with Water (50 mL) and transferred into a separating funnel. The aq. layer was extracted with DCM (3×25 mL). The combined organic layers were collected, washed with brine (100 mL), dried (Na₂SO₄), filtered and concentrated in vacuo to afford the sub-title compound (6.787 g, 24 mmol, 85%).

¹H NMR (500 MHz, DMSO) δ 5.01 (s, 0.5H), 4.96 (s, 0.5H), 4.25-4.16 (m, 2H), 3.63 (s, 2H), 3.60 (s, 1H), 3.48 (ddd, J 10.5, 5.5, 3.9 Hz, 1H), 3.12 (td, J=11.0, 4.0 Hz, 1H), 2.38-2.24 (m, 1H), 1.84 (t, J=4.7 Hz, 0.5H), 1.81 (t, J=4.7 Hz, 0.5H), 1.39 (s, 4H), 1.33 (s, 5H).

### Step 2: 1-(tert-butyl) 2-methyl (2R,4R)-4-methoxypyrrolidine-1,2-dicarboxylate

To a stirred solution the product from step 1 above (1.95 g, 1 Eq, 7.95 mmol) in dry MeCN (39.8 mL) under a nitrogen atmosphere at rt was added silver oxide (7.37 g, 4 Eq, 31.8 mmol) and methyl iodide (11.3 g, 4.97 mL, 10 Eq, 79.5 mmol). The reaction mixture was stirred for 18 hours. The reaction mixture was filtered on Celite, washed with MeCN and the filtrate was concentrated in vacuo to afford the sub-title compound (1.68 g, 6.48 mmol, 81.5%) as a dark yellow oil.

m/z 160.111 (M−Boc+H)⁺ (ES+)

### Step 3: (2R,4R)-1-(tert-butoxycarbonyl)-4-methoxypyrrolidine-2-carboxylic acid

To a solution of the product from step 2 above (1.68 g, 1 Eq, 6.48 mmol) in THF (15.0 mL) was added LiOH (310 mg, 2 Eq, 13.0 mmol) and Water (1.50 mL). The reaction mixture was stirred at room temperature overnight. The mixture was concentrated in vacuo. The pH of the resulting residue was adjusted to 2 with HCl (1.0 M, ~15 mL) then the mixture was extracted with EtOAc (3×100 mL). The combined organic layers were washed with brine (250 mL), dried (MgSO₄), filtered and concentrated in vacuo to give the sub-title compound (1.08 g, 4.40 mmol, 68.0%) as a clear oil.

m/z 145.684 (M−Boc+H)⁺ (ES+)

m/z 244.390 (M−H)⁻ (ES−)

### Step 4: Tert-butyl (2R,4R)-2-(6-fluoro-5-methoxy-1H-indole-3-carbonyl)-4-methoxypyrrolidine-1-carboxylate

To a stirred solution of 6-fluoro-5-methoxy-1H-indole (606 mg, 2 Eq, 3.67 mmol) in dry Toluene (5.00 mL) and THF (5.00 mL) under a nitrogen atmosphere at 22° C. was added MeMgBr in Et₂O (438 mg, 1.22 mL, 3.00 molar, 2 Eq, 3.67 mmol) dropwise over 1 minute. In a separate vial, (the product from step 3 above (450 mg, 1 Eq, 1.83 mmol) dissolved in dry DCM (5.00 mL) under a nitrogen atmosphere was treated with 1-Chloro-NN,2-Trimethylpropenylamine (270 mg, 267 μL, 1.1 Eq, 2.02 mmol, Ghosez reagent). After 30 minutes, the acid chloride solution was added dropwise to the stirring solution of the deprotonated indole. The reaction mixture was stirred for 3 hours. The reaction mixture was quenched with sat. aq. sodium bicarbonate (50 mL) and left to stir at room temperature for 1 hour. The resulting suspension was extracted with EtOAc (3×50 mL), and the collected organic layers were dried (Na₂SO₄), filtered and concentrated in vacuo. The crude product was purified by chromatography on silica gel (80 g cartridge, 25-100% EtOAc/isohexane) to afford the sub-title compound (238 mg, 0.58 mmol, 31%).

m/z 292.918 (M−Boc+H)⁺ (ES+)

¹H NMR (500 MHz, DMSO) δ 11.90 (d, J=11.1 Hz, 1H), 8.32-8.27 (m, 1H), 7.83 (t, J=9.4 Hz, 1H), 7.33 (dd, J=11.3, 2.1 Hz, 1H), 5.08-4.96 (m, 1H), 4.05-3.94 (m, 1H), 3.86 (d, J=3.9 Hz, 3H), 3.78 (dt, J=10.8, 6.8 Hz, 1H), 3.27-3.10 (m, 4H), 2.65 (dq, J=15.5, 8.1 Hz, 1H), 1.76 (dt, J=13.0, 6.5 Hz, 1H), 1.40 (s, 4H), 1.13 (s, 5H).

¹⁹F NMR (471 MHz, DMSO) δ −138.41 (s).

### Step 5: (6-fluoro-5-methoxy-1H-indol-3-yl)((2R,4R)-4-methoxypyrrolidin-2-yl)methanone

To a solution of the product from step 4 above (190 mg, 1 Eq, 484 μmol) in DCM (10.0 mL) and MeOH (2.00 mL) at 22° C. was added HCl in isopropanol (438 mg, 2.00 mL, 6.00 molar, 24.8 Eq, 12.0 mmol) dropwise. The reaction mixture was left to stir for 18 hours. The reaction mixture was concentrated in vacuo to the sub-title compound (140 mg, 0.45 mmol, 94%) as a dark brown oil.

m/z 293.159 (M+H)⁺ (ES+)

### Step 6: 6-fluoro-5-methoxy-3-(((2S,4R)-4-methoxypyrrolidin-2-yl)methyl)-1H-indole

To a stirred solution of the product from step 5 above (140.0 mg, 1 Eq, 478.9 μmol) in dry THF (5.00 mL) under a nitrogen atmosphere at 22° C. was added LiAlH₄ (72.70 mg, 957.9 μL, 2.00 molar, 4 Eq, 1.916 mmol). The reaction mixture was stirred for 2 hours at 66° C. The reaction mixture was cooled down to 0° C. and 3:1 THF:H₂O (5 mL) was added. The white solids were filtered off, the filtrate was further diluted with EtOAc (15 mL), dried (MgSO₄), filtered and concentrated in vacuo. The crude was dissolved in 1.3 mL with DMSO, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge BEH C18 ODB prep column, 130 Å, 5 μm, 30 mm×100 mm, flow rate 40 mL min⁻¹ eluting with a 0.3% Ammonia in water-MeCN gradient over 12.5 mins using UV across all wavelengths with PDA as well as a QDA and ELS detector. At-column dilution pump gives 2 mL min⁻¹ Methanol over the entire method, which is included in the following MeCN percentages. Gradient information: 0.0-0.5 min, 17.5% MeCN; 0.5-10.5 min, ramped from 17.5% MeCN to 47.5% MeCN; 10.5-10.6 min, ramped from 47.5% MeCN to 100% MeCN; 10.6-12.5 min, held at 100% MeCN. The clean fractions were evaporated by freeze drying to obtain the title compound (5.90 mg, 21 mol, 4.4%, 99% Purity) as a clear gum.

m/z 279.662 (M+H)⁺ (ES+)

¹H NMR (500 MHz, DMSO) δ 10.68 (s, 1H), 7.18-7.10 (m, 2H), 7.09 (d, J=2.3 Hz, 1H), 3.82 (s, 4H), 3.15 (s, 4H), 2.88 (dd, J=11.6, 2.7 Hz, 1H), 2.84-2.69 (m, 3H), 1.99 (dt, J=13.5, 7.0 Hz, 1H), 1.32 (ddd, J=12.3, 7.6, 4.1 Hz, 1H). +1 exchangeable proton not observed.

¹⁹F NMR (471 MHz, DMSO) δ −141.48.

### Example 14: Synthesis of (R)-4-(2-((6-fluoro-5-methoxy-1H-indol-3-yl)methyl)pyrrolidin-1-yl)tetrahydro-2H-thiopyran 1,1-dioxide (Compound 3A)

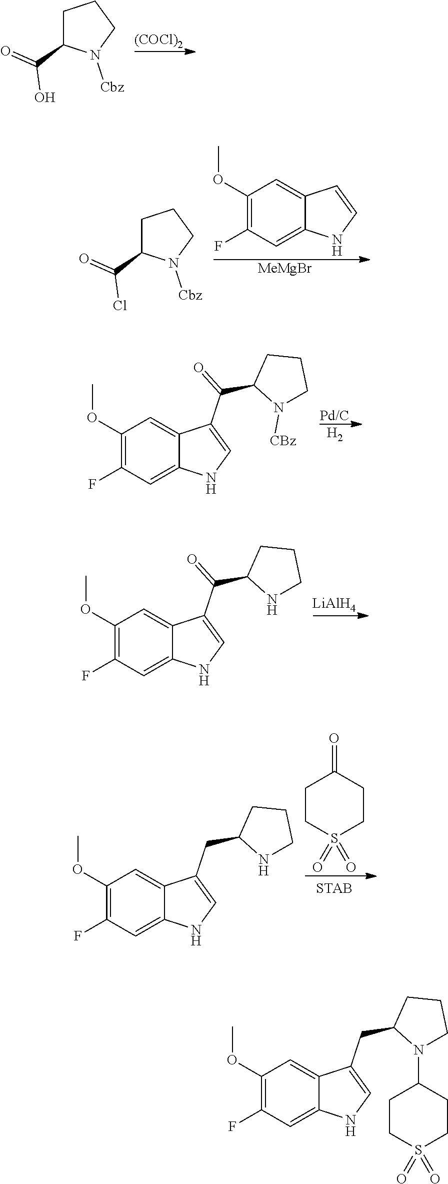

### Step 1: Benzyl (R)-2-(chlorocarbonyl)pyrrolidine-1-carboxylate

To a stirred solution of ((benzyloxy)carbonyl)-D-proline (550 mg, 1 Eq, 2.21 mmol) in DCM (5.50 mL) under a nitrogen atmosphere at 0° C. was added oxalyl dichloride (966 μL, 5 Eq, 11.0 mmol). The reaction mixture was stirred at rt for 4 h. The reaction mixture was concentrated in vacuo, and the resulting crude was suspended in iso-hexane (3×15 mL) and dried in vacuo to provide the sub-title compound (577 mg, 1.9 mmol, 86%) as a pale-yellow oil.

m/z 264.2 (M−Cl+MeOH)⁺ (ES⁺)

### Step 2: Benzyl (R)-2-(6-fluoro-5-methoxy-1H-indole-3-carbonyl)pyrrolidine-1-carboxylate

To a stirred solution of 6-fluoro-5-methoxy-1H-indole (600 mg, 2 Eq, 3.63 mmol) in dry Toluene (10.0 mL) and THF (10.0 mL) under a nitrogen atmosphere at rt was added MeMgBr in diethyl ether (1.21 mL, 3.00 molar, 2 Eq, 3.63 mmol) dropwise over 2 min. The reaction mixture was stirred at rt for 4 h. A solution of product from step 1 above (486 mg, 1 Eq, 1.82 mmol) in dry Toluene (5.0 mL) was added dropwise and the reaction mixture was stirred at rt for 19 h. The reaction mixture was quenched with sat. aq. NaHCO₃ (30 mL) and left to stir at rt for 1 h. The resulting suspension was extracted with EtOAc (3×50 mL), and the collected organic layers were dried over Na₂SO₄ and concentrated in vacuo. The crude product was purified by chromatography on silica gel (80 g cartridge, 25-100% EtOAc/iso-hexane) to afford sub-title compound (1.32 g, 3.2 mmol, 60%, 95% Purity).

m/z 396.6 (M+H)⁺ (ES+); 395.6 (M−H)⁻ (ES−)

### Step 3: (R)-6-Fluoro-5-methoxy-3-prolyl-1H-indole

A stirred solution of the product from step 2 above (233 mg, 1 Eq, 588 μmol) and Pd/C (87 L) (62.5 mg, 5% Wt, 0.05 Eq, 29.4 μmol) in EtOH (5.0 mL) under a nitrogen atmosphere at rt was charged with hydrogen (1 bar). The reaction mixture was stirred at rt for 18 h. The reaction mixture was filtered through glass microfibres, rinsing with MeOH (5 mL). The solvent was removed in vacuo to afford sub-title compound 97.8 mg, 0.32 mmol, 54%) as a dark green oil. Material used without purification.

m/z 262.9 (M+H)⁺ (ES+); 260.9 (M−H)⁻ (ES−)

### Step 4: (R)-6-Fluoro-5-methoxy-3-(pyrrolidin-2-ylmethyl)-1H-indole

To a stirred suspension of the product from step 3 above, combined with previous batch (4235-62), (180 mg, 1 Eq, 686 μmol) in dry THF (5.0 mL) under a nitrogen atmosphere at 0° C. was added LiAlH₄ in THF (1.14 mL, 2.40 molar, 4.0 Eq, 2.75 mmol). The reaction mixture was heated to 60° C. and stirred for 2 h. The reaction mixture was cooled to 0° C. and quenched with 3:1 THF/water (2.5 mL). The mixture was warmed to rt and stirred for 1 h before being filtered. Na₂SO₄ was added to the filtrate and the mixture was filtered and concentrated in vacuo to afford sub-title compound (120 mg, 0.31 mmol, 45%) as a brown gum.

m/z 249.7 (M+H)⁺ (ES+)

### Step 5: (R)-6-fluoro-5-methoxy-3-((1-((3-methoxypyridin-4-yl)methyl)pyrrolidin-2-yl)methyl)-1H-indole, Formic Acid

To a stirred solution of the product from step 4 above (80.0 mg, 1 Eq, 209 μmol) in dry THF (2.00 mL) at 22° C. was added tetrahydro-4H-thiopyran-4-one 1,1-dioxide (46.5 mg, 1.5 Eq, 314 μmol). The reaction mixture was stirred for 1 h. Sodium triacetoxyhydroborate (133 mg, 3 Eq, 628 μmol) was added and the reaction mixture left to stir at rt over the weekend. The reaction mixture was quenched with sat. aq. NaHCO₃ (5 mL) and left to stir for 1 h. The aqueous suspension was diluted with water (5 mL) and extracted with EtOAc (3×10 mL), and the collected organic layers were dried over Na₂SO₄, filtered and concentrated in vacuo. The crude material was dissolved in 1.8 mL with DMSO, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge BEH C18 ODB prep column, 130 Å, 5 μm, 30 mm×100 mm, flow rate 40 mL min-1 eluting with a 0.3% Ammonia in water-MeCN gradient over 17.5 mins using UV across all wavelengths with PDA as well as a QDA and ELS detector. At-column dilution pump gives 2 mL min-1 Methanol over the entire method, which is included in the following MeCN percentages. Gradient information: 0.0-0.5 min, 17.5% MeCN; 0.5-15.5 min, ramped from 17.5% MeCN to 47.5% MeCN; 15.5-15.6 min, ramped from 47.5% MeCN to 100% MeCN; 15.6-17.5 min, held at 100% MeCN. The clean fractions were evaporated in a freeze dryer to afford title compound (10.7 mg, 28 mol, 13%).

m/z 381.2 (M+H)⁺ (ES+); 379.3 (M−H)⁻ (ES−)

¹H NMR (500 MHz, DMSO) δ 10.69 (s, 1H), 7.17-7.07 (m, 3H), 3.83 (s, 3H), 3.27-3.13 (m, 2H), 3.13-3.00 (m, 4H), 2.89-2.78 (m, 2H), 2.59-2.43 (m, 2H), 2.17-2.03 (m, 3H), 1.98 (t, J=10.9 Hz, 1H), 1.71-1.57 (m, 3H), 1.54-1.44 (m, 1H).

¹⁹F NMR (471 MHz, DMSO) δ −141.43.

4235-065 was dissolved in MeOH to 1 mg/mL and screened by SFC (Waters UPC2, with a PDA and a QDA detector). The optimal conditions are using a Lux iC5, 4.6×250 mm, 5 μm, with mobile phase 45% MeOH (28 mM NH3), 55% CO₂. The flow rate was 4 mL/min. The run time was 6 min.

ee=84.10%

### Example 15: Synthesis of 5-methoxy-6-methyl-3-(((R)-pyrrolidin-2-yl)methyl)-3H-indole, formate (Compound 16A)

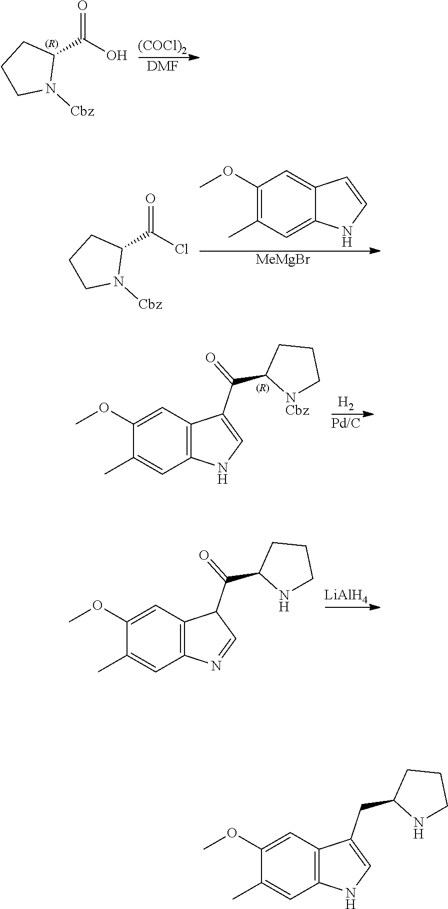

### Step 1: Benzyl (R)-2-(chlorocarbonyl)pyrrolidine-1-carboxylate

To a stirred solution of ((benzyloxy)carbonyl)-D-proline (650 mg, 1 Eq, 2.61 mmol) in dry DCM (8.00 mL) under a nitrogen atmosphere at 0° C. was added oxalyl dichloride (993 mg, 685 L, 3 Eq, 7.82 mmol), followed by 1 drop of DMF. The reaction mixture was stirred for 4 hours at room temperature. The reaction mixture was then concentrated in vacuo and the crude resuspended in toluene (2×15 mL) and dried in vacuo to afford benzyl (R)-2-(chlorocarbonyl)pyrrolidine-1-carboxylate (698 mg, 2.61 mmol, 100%) as a yellow oil.

m/z 264.2 (M−Cl+MeOH)⁺ (ES+)

### Step 2: (R)-2-(5-methoxy-6-methyl-1H-indole-3-carbonyl)pyrrolidine-1-carboxylate

To a stirred solution of 5-methoxy-6-methyl-1H-indole (650 mg, 1.55 Eq, 4.03 mmol) in dry Toluene (12.0 mL) under a nitrogen atmosphere at 22° C. was added methylmagnesium bromide in diethyl ether (483 mg, 1.35 mL, 3.00 molar, 1.55 Eq, 4.05 mmol) dropwise slowly over 5 minutes. THF (1.00 mL) was added to aid solubility.² The reaction mixture was stirred for 4 hours. benzyl (R)-2-(chlorocarbonyl)pyrrolidine-1-carboxylate (698 mg, 1 Eq, 2.61 mmol) in dry Toluene (12.0 mL) was added dropwise and the reaction mixture, THF (1.00 mL) was again added to aid solubility, before the reaction was left to stir at room temperature for 20 hour. The reaction mixture was quenched with sat. sodium bicarbonate (70 mL) and left to stir at room temperature for 30 min. The resulting suspension was extracted with EtOAc (3×60 mL), and the collected organic layers were dried over sodium sulfate and concentrated in vacuo. The crude product was purified by chromatography on silica gel (80 g cartridge, 0-100% EtOAc/isohexane). The columned material was then triturated with EtOAc (4×10 ml) to give a white solid, which was further dried under vacuum to give benzyl (R)-2-(5-methoxy-6-methyl-1H-indole-3-carbonyl)pyrrolidine-1-carboxylate (144 mg, 0.35 mmol, 13%).

m/z 393.2 (M+H)⁺ (ES+)

¹H NMR (500 MHz, DMSO) δ 11.78 (s, 1H), 8.29 (d, J=7.1 Hz, 1H), 7.66 (d, J=20.5 Hz, 1H), 7.38 (d, J=4.6 Hz, 2H), 7.24 (s, 1H), 7.16-7.07 (m, 2H), 7.04 (t, J=7.6 Hz, 1H), 5.06 (q, J=12.9 Hz, 1H), 5.01-4.89 (m, 1H), 4.09 (q, J=5.2 Hz, 1H), 3.81 (d, J=5.2 Hz, 3H), 3.58-3.45 (m, 2H), 2.54 (s, 1H), 2.25 (d, J=4.2 Hz, 3H), 1.87 (dd, J=11.4, 6.8 Hz, 3H).

### Step 3: (R)-5-methoxy-6-methyl-3-prolyl-1H-indole

A stirred suspension of benzyl (R)-2-(5-methoxy-6-methyl-1H-indole-3-carbonyl)pyrrolidine-1-carboxylate (144 mg, 1 Eq, 367 μmol) and palladium on carbon (87 L) (39.0 mg, 5% Wt, 0.05 Eq, 18.3 μmol) in Methanol (10.0 mL) under a hydrogen atmosphere at 1 bar and 22° C. was charged with hydrogen (1.11 mg, 1.5 Eq, 550 μmol). The reaction mixture was stirred at rt overnight. After purging the reaction vessel with N2, the reaction mixture was filtered through glass microfibres, rinsing with MeOH (5 mL). The solvent was removed in vacuo to afford (R)-5-methoxy-6-methyl-3-prolyl-1H-indole (88.1 mg, 0.32 mmol, 86%).

m/z 258.9 (M+H)⁺ (ES+)

¹H NMR (500 MHz, DMSO) δ 11.77 (s, 1H), 8.22 (s, 1H), 7.66 (s, 1H), 7.23 (d, J=1.1 Hz, 1H), 4.35 (dd, J=8.6, 6.1 Hz, 1H), 3.81 (s, 3H), 3.09-3.00 (m, 1H), 2.74 (dt, J=10.4, 6.6 Hz, 1H), 2.24 (s, 3H), 2.15 (td, J=8.3, 6.0 Hz, 1H), 1.72 (t, J=7.0 Hz, 1H), 1.67-1.56 (m, 2H). Missing 1 exchangeable proton.

### Step 3: (R)-5-methoxy-6-methyl-3-(pyrrolidin-2-ylmethyl)-1H-indole, Formate

To a stirred solution of (R)-5-methoxy-6-methyl-3-prolyl-1H-indole (88.0 mg, 1 Eq, 341 μmol) in dry 2-Methyltetrahydrofuran (10.0 mL) under a nitrogen atmosphere at 0° C. was added LiAlH₄ in THF (50.9 mg, 670 μL, 2.00 molar, 3.93 Eq, 1.34 mmol). The reaction mixture was heated to 66° C. for 3 hours. The heating was stopped and the reaction stirred at room temperature overnight. The reaction mixture was cooled to 0° C. and quenched using a 1:1 mixture of Glauber's salt (Na₂SO₄·10H₂O) and Celite added portion wise. The mixture was then filtered and the solid washed with TBME (2×20 ml). The filtrate was dried over MgSO₄ and concentrated in vacuo to give a clear orange viscous liquid. The crude product was purified by chromatography on RP Flash C18 (24 g cartridge, 0-100% (0.1% Formic acid in MeCN)/(0.1% Formic Acid in Water)) to afford the desired product as a sticky orange oil. The sticky oil was triturated with MeCN afford (R)-5-methoxy-6-methyl-3-(pyrrolidin-2-ylmethyl)-1H-indole, Formate (45.2 mg, 0.15 mmol, 45%).

m/z 245.2 (M+H)⁺ (ES+)

¹H NMR (500 MHz, DMSO) δ 10.59 (s, 1H), 8.42 (s, 1H), 7.09 (d, J=5.8 Hz, 2H), 7.00 (d, J=3.2 Hz, 1H), 3.79 (s, 3H), 3.57 (s, 1H), 3.11 (s, 1H), 2.98 (s, 2H), 2.85 (dd, J=13.9, 7.3 Hz, 1H), 2.22 (s, 3H), 1.90 (d, J=12.5 Hz, 2H), 1.81-1.72 (m, 1H), 1.54 (s, 1H). Missing NH proton.

The material was screened by SFC (Waters UPC², with a PDA and a QDA detector). The optimal conditions are using a Chiralpak IH column, 4.6×250 mm, 5 μm, with mobile phase 20% Methanol (28 mM NH₃), 80% CO₂. The flow rate was 4 mL/min. The run time was 4.5 mins. EE was 91.82%.

### Example 16: Synthesis of benzyl (2R)-2-((5-methoxy-6-methyl-3H-indol-3-yl)methyl)pyrrolidine-1-carboxylate, formate (Compound 6B)

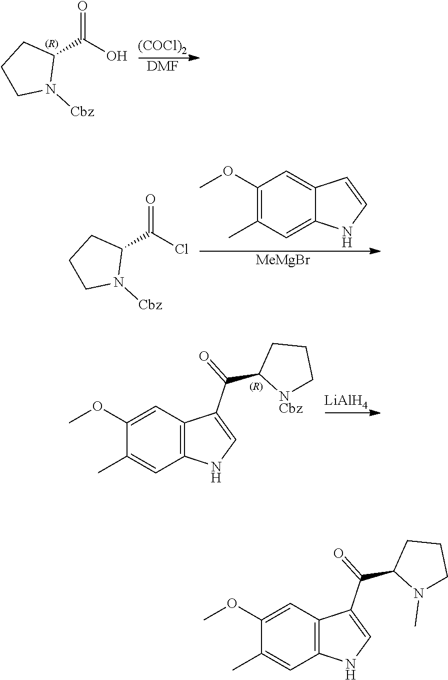

### Step 1: Benzyl (R)-2-(chlorocarbonyl)pyrrolidine-1-carboxylate

To a stirred solution of ((benzyloxy)carbonyl)-D-proline (650 mg, 1 Eq, 2.61 mmol) in dry DCM (8.00 mL) under a nitrogen atmosphere at 0° C. was added oxalyl dichloride (993 mg, 685 L, 3 Eq, 7.82 mmol), followed by 1 drop of DMF. The reaction mixture was stirred for 4 hours at room temperature. The reaction mixture was then concentrated in vacuo and the crude resuspended in toluene (2×15 mL) and dried in vacuo to afford benzyl (R)-2-(chlorocarbonyl)pyrrolidine-1-carboxylate (698 mg, 2.61 mmol, 100%) as a yellow oil.

m/z 264.2 (M−Cl+MeOH)⁺ (ES+)

### Step 2: Benzyl 2-(3-(((benzyloxy)carbonyl)-D-prolyl)-5-methoxy-6-methyl-1H-indole-1-carbonyl)pyrrolidine-1-carboxylate

To a stirred solution of 5-methoxy-6-methyl-1H-indole (650 mg, 1.55 Eq, 4.03 mmol) in dry Toluene (12.0 mL) under a nitrogen atmosphere at 22° C. was added methylmagnesium bromide in diethyl ether (483 mg, 1.35 mL, 3.00 molar, 1.55 Eq, 4.05 mmol) dropwise slowly over 5 minutes. THF (1.00 mL) was added to aid solubility.² The reaction mixture was stirred for 4 hours. benzyl (R)-2-(chlorocarbonyl)pyrrolidine-1-carboxylate (698 mg, 1 Eq, 2.61 mmol) in dry Toluene (12.0 mL) was added dropwise and the reaction mixture, THF (1.00 mL) was again added to aid solubility, before the reaction was left to stir at room temperature for 20 hour. The reaction mixture was quenched with sat. sodium bicarbonate (70 mL) and left to stir at room temperature for 30 min. The resulting suspension was extracted with EtOAc (3×60 mL), and the collected organic layers were dried over sodium sulfate and concentrated in vacuo. The crude product was purified by chromatography on silica gel (80 g cartridge, 0-100% EtOAc/isohexane). The column material was then triturated with EtOAc (4×10 ml). The liquor from the trituration was then combined and concentrated in vacuum to give benzyl 2-(3-(((benzyloxy)carbonyl)-D-prolyl)-5-methoxy-6-methyl-1H-indole-1-carbonyl)pyrrolidine-1-carboxylate (723.4 mg, 1.0 mmol, 40%).

m/z 580.3 (M+H)⁺ (ES+)

### Step 3: benzyl (2R)-2-((5-methoxy-6-methyl-3H-indol-3-yl)methyl)pyrrolidine-1-carboxylate

To a stirred solution of benzyl 3-(((benzyloxy)carbonyl)-D-prolyl)-5-methoxy-6-methyl-1H-indole-1-carboxylate (100 mg, 1 Eq, 190 μmol) in dry 2-Methyltetrahydrofuran (6.00 mL) under a nitrogen atmosphere at 0° C. was added LiAlH₄ in THF (43.2 mg, 570 μL, 2.00 molar, 6 Eq, 1.14 mmol). The reaction mixture was heated to 66° C. for 3 hours. The heating was stopped and the reaction stirred at room temperature overnight. The reaction mixture was cooled to 0° C. and quenched using a 1:1 mixture of Glauber's salt (Na₂SO₄·10H₂O) and Celite added portion wise. The mixture was then filtered and the solid washed with TBME (2×20 ml). The filtrate was dried over MgSO₄ and concentrated in vacuo to give a pale brown gum. The crude product was purified by chromatography on RP Flash C18 (24 g cartridge, 0-100% (0.1% Formic acid in MeCN)/(0.1% Formic Acid in Water)) to afford (R)-5-methoxy-6-methyl-3-((1-methylpyrrolidin-2-yl)methyl)-1H-indole, Formic Acid (36.6 mg, 0.11 mmol, 60%).

m/z 259.3 (M+H)⁺ (ES+); 259.2 (M−H)⁻ (ES−)

¹H NMR (500 MHz, DMSO) δ 10.49 (s, 1H), 8.23 (s, 1H), 7.08 (d, J=1.0 Hz, 1H), 7.00 (d, J=2.3 Hz, 1H), 6.93 (s, 1H), 3.78 (s, 3H), 3.12-3.06 (m, 1H), 3.03 (dd, J=13.3, 3.1 Hz, 1H), 2.62-2.51 (m, 2H), 2.43 (s, 3H), 2.29 (q, J=9.0 Hz, 1H), 2.21 (d, J=0.8 Hz, 3H), 1.77 (ddd, J=16.6, 12.5, 8.2 Hz, 1H), 1.72-1.57 (m, 2H), 1.57-1.46 (m, 1H).

The material was screened by SFC (Waters UPC², with a PDA and a QDA detector). The optimal conditions are using a Chiralpak IG column, 4.6×250 mm, 5 μm, with mobile phase 30% Methanol (28 mM NH₃), 70% CO₂. The flow rate was 4 mL/min. The run time was 4.5 mins.

ee was 95.23%.

### Example 17: Synthesis of (R)-7-((1-methylpyrrolidin-2-yl)methyl)-5H-[1,3]dioxolo[4,5-f]indole, Formic Acid (Compound 15A)

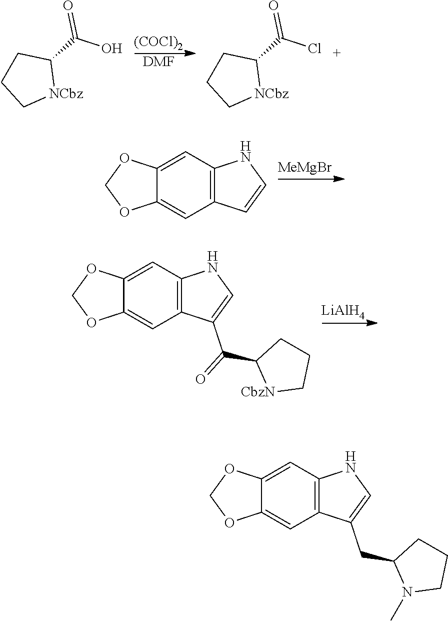

### Step 1: Benzyl (R)-2-(chlorocarbonyl)pyrrolidine-1-carboxylate

To a stirred solution of ((benzyloxy)carbonyl)-D-proline (2.59 g, 10.4 mmol) in dry CH₂Cl₂ (35.0 mL) under a nitrogen atmosphere at 0° C. was added oxalyl dichloride (4.50 mL, 51.4 mmol) and two drops of DMF dropwise. The reaction mixture was stirred at 22° C. for 3 hours. The reaction mixture was concentrated in vacuo resuspended in isohexane (5×50 mL) and dried in vacuo to afford benzyl (R)-2-(chlorocarbonyl)pyrrolidine-1-carboxylate (2.78 g, 10 mmol, 99%) as a dark yellow oil.

The product was used without analysis.

### Step 2: Benzyl (R)-2-(5H-[1,3]dioxolo[4,5-f]indole-7-carbonyl)pyrrolidine-1-carboxylate

To a stirred solution of 5H-[1,3]dioxolo[4,5-f]indole (3.00 g, 18.6 mmol) in dry PhMe (60.0 mL) under a nitrogen atmosphere at 22° C. was added MeMgBr in Et₂O (6.30 mL, 3.00 molar, 18.9 mmol). The reaction mixture was stirred for 3 hours. A solution of benzyl (R)-2-(chlorocarbonyl)pyrrolidine-1-carboxylate (2.49 g, 9.31 mmol) in dry PhMe (60.0 mL) was added dropwise at 22° C. and the reaction mixture was left to stir for 18 hours. The reaction mixture was quenched with water (100 mL) diluted with brine (100 mL) and the resulting biphasic solution was extracted with EtOAc (2×250 mL). The collected organic layers were dried over magnesium sulfate and concentrated in vacuo.

The crude product was purified by chromatography on silica gel (120 g cartridge, 0-100% EtOAc/isohexane) to afford benzyl (R)-2-(5H-[1,3]dioxolo[4,5-f]indole-7-carbonyl)pyrrolidine-1-carboxylate (2.50 g, 5.7 mmol, 62%).

m/z 393.6 (M+H)⁺ (ES+)

¹H NMR (500 MHz, DMSO) δ 11.86-11.80 (m, 1H), 8.25 (dd, J=12.7, 3.1 Hz, 1H), 7.59 (d, J=21.2 Hz, 1H), 7.42-7.28 (m, 3H), 7.17-6.95 (m, 3H), 6.00 (dd, J=4.9, 4.9 Hz, 2H), 5.21-5.11 (m, 1H), 5.11-5.02 (m, 1H), 5.00-4.89 (m, 1H), 3.61-3.44 (m, 2H), 2.42-2.24 (m, 1H), 1.93-1.77 (m, 3H).

### Step 3: (R)-7-((1-methylpyrrolidin-2-yl)methyl)-5H-[1,3]dioxolo[4,5-f]indole, Formic Acid

To a solution of benzyl (R)-2-(5H-[1,3]dioxolo[4,5-f]indole-7-carbonyl)pyrrolidine-1-carboxylate (434 mg, 995 μmol) in dry 2-MeTHF (5.00 mL) under a nitrogen atmosphere at 0° C. was added LiAlH₄ in THF (2.00 mL, 2.00 molar, 4.00 mmol) dropwise. The reaction mixture was heated to 65° C. and left to stir for 18 hours. The reaction mixture was cooled to 0° C. and quenched with 3:1 THF/water (20 mL). The resulting solids were removed via filtration and washed with THF (3×10 mL). The filtrate was dried over magnesium sulfate and concentrated in vacuo.

The crude product was purified by chromatography on RP Flash C18 (24 g cartridge, 0-100% (0.1% Formic acid in MeCN)/(0.1% Formic Acid in Water)) to afford (R)-7-((1-methylpyrrolidin-2-yl)methyl)-5H-[1,3]dioxolo[4,5-f]indole, Formic Acid (191.5 mg, 0.60 mmol, 60%).

m/z 259.6 (M+H)⁺ (ES+)

¹H NMR (500 MHz, DMSO) δ 10.66 (s, 1H), 8.29 (s, 1H), 7.02-6.98 (m, 2H), 6.86 (s, 1H), 5.91 (d, J=5.7 Hz, 2H), 3.27-3.19 (m, 1H), 3.07 (dd, J=14.0, 4.2 Hz, 1H), 2.90-2.81 (m, 1H), 2.60 (dd, J=14.1, 9.5 Hz, 1H), 2.55-2.50 (m, 4H), 1.86-1.66 (m, 3H), 1.61-1.50 (m, 1H).

1 proton not observed due to exchange.

ee 99%.

### Example 18: Synthesis of (R)-6-fluoro-5-methoxy-3-((1-((1-methyl-1H-pyrazol-3-yl)methyl)pyrrolidin-2-yl)methyl)-1H-indole, Formate (Compound 5B)

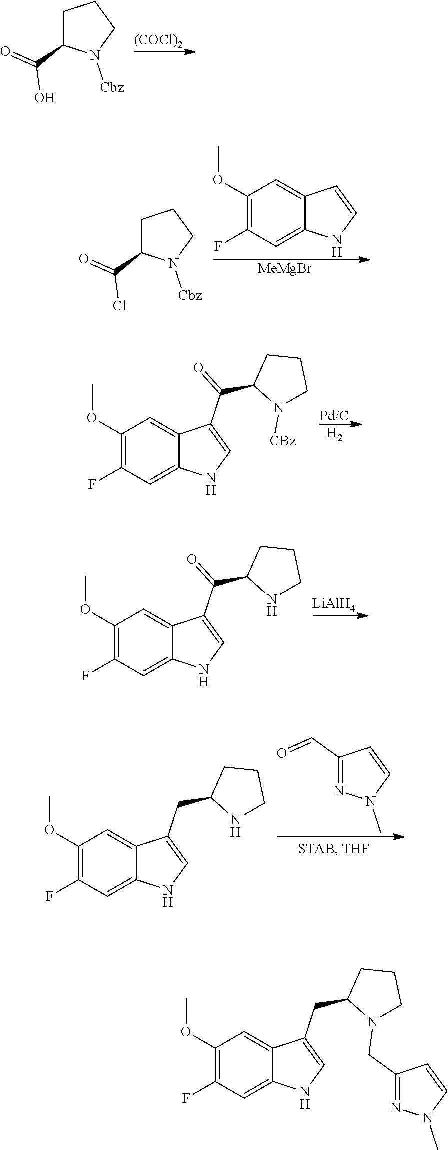

### Step 1: Benzyl (R)-2-(chlorocarbonyl)pyrrolidine-1-carboxylate

To a stirred solution of ((benzyloxy)carbonyl)-D-proline (1.32 g, 1 Eq, 5.30 mmol) in DCM (15.0 mL) under a nitrogen atmosphere at 0° C. was added oxalyl dichloride (3.33 g, 2.30 mL, 4.96 Eq, 26.3 mmol). The reaction mixture was stirred at rt for 3 h. The reaction mixture was concentrated in vacuo, and the resulting crude was suspended in iso-hexane (3×15 mL) and dried in vacuo to provide the sub-title compound (1.42 g, 5.30 mmol, 100%) as a pale-yellow oil.

m/z 264.2 (M−Cl+MeOH)⁺ (ES⁺)

### Step 2: Benzyl (R)-2-(6-fluoro-5-methoxy-1H-indole-3-carbonyl)pyrrolidine-1-carboxylate

To a stirred solution of 6-fluoro-5-methoxy-1H-indole (1.75 g, 2 Eq, 10.6 mmol) in dry Toluene (10.0 mL) under a nitrogen atmosphere at rt was added MeMgBr in diethyl ether (1.26 g, 3.54 mL, 3.00 molar, 2 Eq, 10.6 mmol) dropwise over 2 min. THF (4 mL) was added. The reaction mixture was stirred at rt for 4 h. A solution of product from step 1 above (1.42 g, 1 Eq, 5.30 mmol) in dry Toluene (15.0 mL) was added dropwise and the reaction mixture was stirred at rt for 72 h. The reaction mixture was quenched with sat. aq. NaHCO₃ (50 mL) and left to stir at rt for 1 h. The resulting suspension was extracted with EtOAc (3×50 mL), and the collected organic layers were dried over Na₂SO₄ and concentrated in vacuo. The crude product was purified by chromatography on silica gel (80 g cartridge, 25-100% EtOAc/iso-hexane) to afford sub-title compound (1.32 g, 3.2 mmol, 60%, 95% Purity).

m/z 397.1 (M+H)⁺ (ES+); 395.6 (M−H)⁻ (ES−)

¹H NMR (500 MHz, DMSO) δ 11.95 (s, 1H), 8.39 (d, J=10.6 Hz, 1H), 7.89-7.80 (m, 1H), 7.42-7.31 (m, 3H), 7.17-7.00 (m, 3H), 5.24-5.12 (m, 1H), 5.10-5.00 (m, 1H), 4.94 (q, J=13.2 Hz, 1H), 3.86 (s, 3H), 3.56-3.47 (m, 2H), 2.42-2.28 (m, 2H), 1.91-1.80 (m, 2H).

¹⁹F NMR (471 MHz, DMSO) δ −138.33 (m).

### Step 3: (R)-6-Fluoro-5-methoxy-3-prolyl-1H-indole

A stirred solution of the product from step 2 above (1.32 g, 1 Eq, 3.33 mmol) and Pd/C (87 L) (354 mg, 5% Wt, 0.05 Eq, 166 μmol) in MeOH (100.0 mL) under a nitrogen atmosphere at rt was charged with hydrogen (1 bar). The reaction mixture was stirred at rt for 18 h. The reaction mixture was filtered through glass microfibres, rinsing with MeOH (5 mL). The solvent was removed in vacuo to afford sub-title compound (840 mg, 3.0 mmol, 91%).

m/z 263.5 (M+H)⁺ (ES+); 261.0 (M−H)⁻ (ES−)

¹H NMR (500 MHz, DMSO) δ 8.34 (s, 1H), 7.84 (d, J=8.7 Hz, 1H), 7.33 (d, J=11.4 Hz, 1H), 4.40 (dd, J=8.7, 6.0 Hz, 1H), 3.86 (s, 3H), 3.06 (dt, J=11.6, 6.2 Hz, 1H), 2.78 (dt, J=10.4, 6.5 Hz, 1H), 2.22-2.11 (m, 1H), 1.77-1.69 (m, 1H), 1.69-1.59 (m, 2H). Two exchangeable protons not observed.

¹⁹F NMR (471 MHz, DMSO) δ −138.43.

### Step 4: (R)-6-Fluoro-5-methoxy-3-(pyrrolidin-2-ylmethyl)-1H-indole

To a stirred suspension of the product from step 3 above (835 mg, 1 Eq, 3.18 mmol) in dry THF (30.0 mL) under a nitrogen atmosphere at 0° C. was added LiAlH₄ in THF (6.50 mL, 2.00 molar, 4.08 Eq, 13.0 mmol). The reaction mixture was heated to 66° C. and stirred for 2 h. The reaction mixture was cooled to 0° C. and quenched with 3:1 THF/water (16 mL). The mixture was warmed to rt and stirred for 1 h before being filtered. Na₂SO₄ was added to the filtrate and the mixture was filtered and concentrated in vacuo to afford sub-title compound (750 mg, 1.9 mmol, 61%) as a brown gum.

m/z 249.5 (M+H)⁺ (ES+)

### Step 5: (R)-6-fluoro-5-methoxy-3-((1-((1-methyl-1H-pyrazol-3-yl)methyl)pyrrolidin-2-yl)methyl)-1H-indole

To a stirred solution of (R)-6-fluoro-5-methoxy-3-(pyrrolidin-2-ylmethyl)-1H-indole (106.7 mg, 64% Wt, 1 Eq, 275.0 μmol) in dry Tetrahydrofuran (3.00 mL) at 22° C. was added 1-methyl-1H-pyrazole-3-carbaldehyde (60.0 mg, 1.98 Eq, 545 μmol). The reaction mixture was stirred for 10 min. Sodium triacetoxyhydroborate (230 mg, 3.95 Eq, 1.09 mmol) was then added and the reaction mixture left to stir for 18 h. The reaction mixture was quenched with sat. sodium bicarbonate solution (10 mL) and left to stir for 15 min. The aqueous suspension was then extracted with EtOAc (3×15 mL), and the combined organic layers were washed with brine (50 mL), dried over sodium sulfate and concentrated in vacuo. The crude product was initially purified by chromatography on RP Flash C18 (24 g cartridge, 5-20% (0.1% Formic acid in MeCN)/(0.1% Formic Acid in Water)). The material was dissolved in 1.7 mL with DMSO, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge BEH C18 ODB prep column, 130 Å, 5 μm, 30 mm×100 mm, flow rate 40 mL min⁻¹ eluting with a 0.3% Ammonia in water-MeCN gradient over 17.5 mins using UV across all wavelengths with PDA as well as a QDA and ELS detector. At-column dilution pump gives 2 mL min⁻¹ Methanol over the entire method, which is included in the following MeCN percentages. Gradient information: 0.0-0.5 min, 20% MeCN; 0.5-15.5 min, ramped from 20% MeCN to 50% MeCN; 15.5-15.6 min, ramped from 50% MeCN to 100% MeCN; 15.6-17.5 min, held at 100% MeCN. The clean fractions were evaporated in a Genevac. The dried fractions were then combined using EtOAc, before being concentrated and dried in a desiccator for 1 hour to give (R)-6-fluoro-5-methoxy-3-((1-((1-methyl-1H-pyrazol-3-yl)methyl)pyrrolidin-2-yl)methyl)-1H-indole (38.8 mg, 0.11 mmol).

m/z 343.2 (M+H)⁺ (ES+)

¹H NMR (500 MHz, DMSO) δ 10.66 (s, 1H), 7.58 (d, J=2.1 Hz, 1H), 7.17 (d, J=8.5 Hz, 1H), 7.12 (d, J=11.8 Hz, 1H), 7.08 (d, J=2.3 Hz, 1H), 6.13 (d, J=2.2 Hz, 1H), 3.95 (d, J=13.4 Hz, 1H), 3.83 (s, 3H), 3.79 (s, 3H), 3.45 (d, J=13.4 Hz, 1H), 3.02 (dd, J=13.9, 3.6 Hz, 1H), 2.89 (ddd, J=9.7, 7.1, 2.7 Hz, 1H), 2.76-2.65 (m, 1H), 2.26 (td, J=9.1, 7.6 Hz, 1H), 1.65 (ddt, J=17.0, 13.3, 6.6 Hz, 1H), 1.60-1.49 (m, 2H), 1.49-1.39 (m, 1H). Missing one proton, which is hidden beneath DMSO peak. Confirmed by HSQC.

¹⁹F NMR (471 MHz, DMSO) δ −141.43 (dd, J=11.9, 8.5 Hz). (no decoupling)

The material was screened by SFC (Waters UPC², with a PDA and a QDA detector). The optimal conditions are using a Lux A1 column, 4.6×250 mm, 5 μm, with mobile phase 20% Methanol (28 mM NH₃), 80% CO₂. The flow rate was 4 mL/min. The run time was 5.0 mins. EE 49.76%.

### Example 19: Synthesis of (R)-7-((1-isopropylpyrrolidin-2-yl)methyl)-5H-dioxolo[4,5-f]indole, Formic Acid (Compound 17A)

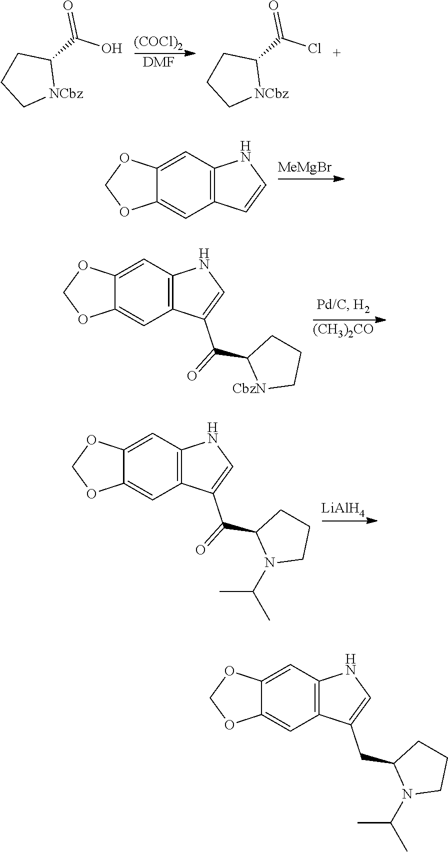

### Step 1: Benzyl (R)-2-(chlorocarbonyl)pyrrolidine-1-carboxylate

To a stirred solution of ((benzyloxy)carbonyl)-D-proline (2.59 g, 10.4 mmol) in dry CH₂Cl₂ (35.0 mL) under a nitrogen atmosphere at 0° C. was added oxalyl dichloride (4.50 mL, 51.4 mmol) and two drops of DMF dropwise. The reaction mixture was stirred at 22° C. for 3 hours. The reaction mixture was concentrated in vacuo resuspended in isohexane (5×50 mL) and dried in vacuo to afford benzyl (R)-2-(chlorocarbonyl)pyrrolidine-1-carboxylate (2.78 g, 10 mmol, 99%) as a dark yellow oil.

The product was used without analysis.

### Step 2: Benzyl (R)-2-(6-chloro-5-methoxy-1H-indole-3-carbonyl)pyrrolidine-1-carboxylate

To a stirred solution of 5H-[1,3]dioxolo[4,5-f]indole (3.00 g, 18.6 mmol) in dry PhMe (60.0 mL) under a nitrogen atmosphere at 22° C. was added MeMgBr in Et₂O (6.30 mL, 3.00 molar, 18.9 mmol). The reaction mixture was stirred for 3 hours. A solution of benzyl (R)-2-(chlorocarbonyl)pyrrolidine-1-carboxylate (2.49 g, 9.31 mmol) in dry PhMe (60.0 mL) was added dropwise at 22° C. and the reaction mixture was left to stir for 18 hours. The reaction mixture was quenched with water (100 mL) diluted with brine (100 mL) and the resulting biphasic solution was extracted with EtOAc (2×250 mL). The collected organic layers were dried over magnesium sulfate and concentrated in vacuo.

The crude product was purified by chromatography on silica gel (120 g cartridge, 0-100% EtOAc/isohexane) to afford benzyl (R)-2-(5H-[1,3]dioxolo[4,5-f]indole-7-carbonyl)pyrrolidine-1-carboxylate (2.50 g, 5.7 mmol, 62%).

m/z 393.6 (M+H)⁺ (ES+)

¹H NMR (500 MHz, DMSO) δ 11.86-11.80 (m, 1H), 8.25 (dd, J=12.7, 3.1 Hz, 1H), 7.59 (d, J=21.2 Hz, 1H), 7.42-7.28 (m, 3H), 7.17-6.95 (m, 3H), 6.00 (dd, J=4.9, 4.9 Hz, 2H), 5.21-5.11 (m, 1H), 5.11-5.02 (m, 1H), 5.00-4.89 (m, 1H), 3.61-3.44 (m, 2H), 2.42-2.24 (m, 1H), 1.93-1.77 (m, 3H).

### Step 3: 7-(isopropyl-D-prolyl)-5H-[1,3]dioxolo[4,5-f]indole

A solution of benzyl (R)-2-(5H-[1,3]dioxolo[4,5-f]indole-7-carbonyl)pyrrolidine-1-carboxylate (2.00 g, 5.10 mmol), acetone (1.00 mL, 13.60 mmol) and palladium on carbon (600 mg, 10% Wt, 564 μmol) in MeOH (40.0 mL) under a nitrogen atmosphere at 22° C. was charged with hydrogen gas to a pressure of 2 bar. The reaction mixture was left to stir for 3 hours. The reaction mixture was filtered through glass microfibres and concentrated in vacuo to afford 7-(isopropyl-D-prolyl)-5H-[1,3]dioxolo[4,5-f]indole (1.78 g, 5.0 mmol, 99%).

m/z 301.5 (M+H)⁺ (ES+)

¹H NMR (500 MHz, DMSO) δ 11.71 (s, 1H), 8.39 (s, 1H), 7.61 (s, 1H), 6.99 (s, 1H), 5.98 (s, 2H), 3.94 (dd, J=9.4, 5.4 Hz, 1H), 3.15-3.07 (m, 1H), 2.82 (hept, J=6.4 Hz, 1H), 2.64-2.56 (m, 1H), 2.12 (dq, J=12.2, 8.7 Hz, 1H), 1.88-1.66 (m, 3H), 0.97 (dd, J=6.4, 4.0 Hz, 6H).

### Step 4: (R)-7-((1-isopropylpyrrolidin-2-yl)methyl)-5H-[1,3]dioxolo[4,5-f]indole, Formic Acid

To a solution of 7-(isopropyl-D-prolyl)-5H-[1,3]dioxolo[4,5-f]indole (300 mg, 999 μmol) in dry 2-MeTHF (5.00 mL) under a nitrogen atmosphere at 0° C. was added LiAlH₄ in THF (2.00 mL, 2.00 molar, 4.00 mmol) dropwise. The reaction mixture was heated to 65° C. and left to stir for 4 hours. The reaction mixture was cooled to 0° C. and quenched with 3:1 THF/water (20 mL). The solution was dried over magnesium sulfate, filtered and concentrated in vacuo.

The crude product was purified by chromatography on RP Flash C18 (24 g cartridge, 0-100% (0.1% Formic acid in MeCN)/(0.1% Formic Acid in Water)) to afford (R)-7-((1-isopropylpyrrolidin-2-yl)methyl)-5H-[1,3]dioxolo[4,5-f]indole, Formic Acid (248 mg, 0.73 mmol, 73%) as a dark brown oil.

m/z 287.2 (M+H)⁺ (ES+)

¹H NMR (500 MHz, DMSO) δ 10.63 (s, 1H), 8.25 (s, 1H), 7.00 (d, J=2.3 Hz, 1H), 6.93 (s, 1H), 6.85 (s, 1H), 5.91 (dd, J=6.5, 0.9 Hz, 2H), 3.21 (dt, J=13.3, 6.6 Hz, 2H), 2.98 (d, J=8.9 Hz, 1H), 2.90 (dd, J=14.1, 3.9 Hz, 1H), 2.69 (d, J=8.7 Hz, 1H), 2.58-2.52 (m, 1H), 1.78-1.60 (m, 3H), 1.54 (dd, J=9.2, 4.0 Hz, 1H), 1.25-0.98 (m, 6H).

1 proton not observed due to exchange.

### Example 20: Synthesis of (R)-7-(pyrrolidin-2-ylmethyl)-5H-[1,3]dioxolo[4,5-f]indole, formate (Compound 18A)

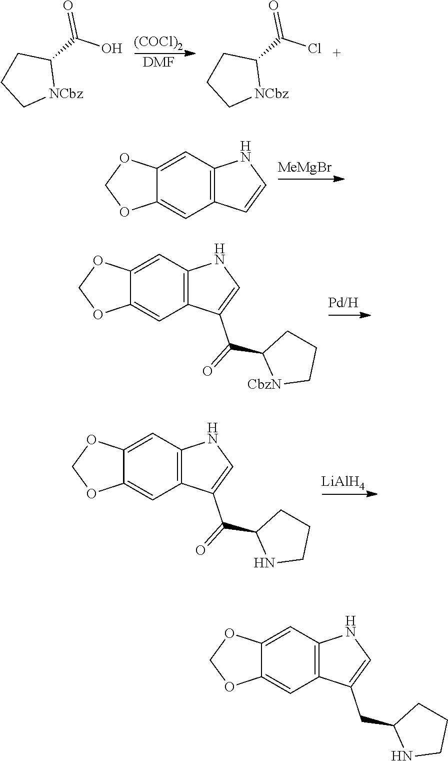

### Step 1: Benzyl (R)-2-(chlorocarbonyl)pyrrolidine-1-carboxylate

To a stirred solution of ((benzyloxy)carbonyl)-D-proline (2.59 g, 10.4 mmol) in dry CH₂Cl₂ (35.0 mL) under a nitrogen atmosphere at 0° C. was added oxalyl dichloride (4.50 mL, 51.4 mmol) and two drops of DMF dropwise. The reaction mixture was stirred at 22° C. for 3 hours. The reaction mixture was concentrated in vacuo resuspended in isohexane (5×50 mL) and dried in vacuo to afford benzyl (R)-2-(chlorocarbonyl)pyrrolidine-1-carboxylate (2.78 g, 10 mmol, 99%) as a dark yellow oil.

The product was used without analysis.

### Step 2: Benzyl (R)-2-(5H-[1,3]dioxolo[4,5-f]indole-7-carbonyl)pyrrolidine-1-carboxylate

To a stirred solution of 5H-[1,3]dioxolo[4,5-f]indole (3.00 g, 18.6 mmol) in dry PhMe (60.0 mL) under a nitrogen atmosphere at 22° C. was added MeMgBr in Et₂O (6.30 mL, 3.00 molar, 18.9 mmol). The reaction mixture was stirred for 3 hours. A solution of benzyl (R)-2-(chlorocarbonyl)pyrrolidine-1-carboxylate (2.49 g, 9.31 mmol) in dry PhMe (60.0 mL) was added dropwise at 22° C. and the reaction mixture was left to stir for 18 hours. The reaction mixture was quenched with water (100 mL) diluted with brine (100 mL) and the resulting biphasic solution was extracted with EtOAc (2×250 mL). The collected organic layers were dried over magnesium sulfate and concentrated in vacuo.

The crude product was purified by chromatography on silica gel (120 g cartridge, 0-100% EtOAc/isohexane) to afford benzyl (R)-2-(5H-[1,3]dioxolo[4,5-f]indole-7-carbonyl)pyrrolidine-1-carboxylate (2.50 g, 5.7 mmol, 62%).

m/z 393.6 (M+H)⁺ (ES+)

¹H NMR (500 MHz, DMSO) δ 11.86-11.80 (m, 1H), 8.25 (dd, J=12.7, 3.1 Hz, 1H), 7.59 (d, J=21.2 Hz, 1H), 7.42-7.28 (m, 3H), 7.17-6.95 (m, 3H), 6.00 (dd, J=4.9, 4.9 Hz, 2H), 5.21-5.11 (m, 1H), 5.11-5.02 (m, 1H), 5.00-4.89 (m, 1H), 3.61-3.44 (m, 2H), 2.42-2.24 (m, 1H), 1.93-1.77 (m, 3H).

### Step 3: (R)-7-prolyl-5H-[1,3]dioxolo[4,5-f]indole

A solution of benzyl (R)-2-(5H-[1,3]dioxolo[4,5-f]indole-7-carbonyl)pyrrolidine-1-carboxylate (585 mg, 1 Eq, 1.49 mmol) and palladium on carbon (159 mg, 10% Wt, 0.1 Eq, 149 mol) in Methanol (10.0 mL) under a nitrogen atmosphere at 22° C. was charged with hydrogen (3.01 mg, 1 Eq, 1.49 mmol) gas to a pressure of 2 bar. The reaction mixture was left to stir for 18 hours. The reaction mixture was filtered through glass microfibres and concentrated in vacuo to afford (R)-7-prolyl-5H-[1,3]dioxolo[4,5-f]indole (385 mg, 1.2 mmol, 82%).

m/z 259.3 (M+H)⁺ (ES+)

1H NMR (500 MHz, DMSO) δ 11.85 (s, 1H), 8.20 (d, J=1.4 Hz, 1H), 7.58 (s, 1H), 7.00 (s, 1H), 5.99 (s, 2H), 4.38 (ddd, J=8.6, 6.2, 2.4 Hz, 1H), 3.69-3.61 (m, 1H), 3.11-3.01 (m, 1H), 2.81-2.72 (m, 1H), 2.22-2.10 (m, 1H), 1.72 (q, J=7.3 Hz, 1H), 1.68-1.55 (m, 2H).

### Step 4: (R)-7-(pyrrolidin-2-ylmethyl)-5H-[1,3]dioxolo[4,5-f]indole, Formate

To a stirred solution of (R)-7-prolyl-5H-[1,3]dioxolo[4,5-f]indole (385 mg, 1 Eq, 1.49 mmol) in dry 2-Methyltetrahydrofuran (6.00 mL) under a nitrogen atmosphere at 0° C. was added LiAlH4 in THF (226 mg, 2.98 mL, 2.00 molar, 4 Eq, 5.96 mmol). The reaction mixture was heated to 66° C. for 4 hours.¹ The heating was stopped and the reaction stirred at room temperature overnight. The reaction mixture was cooled to 0° C. and quenched using a 1:1 mixture of Glauber's salt (Na2SO4·10H2O) and Celite added portion wise. The mixture was then filtered and the solid washed with TBME (2×20 ml). The filtrate was dried over MgSO4 and concentrated in vacuo to give a pale brown gum. The crude product was purified by chromatography on RP Flash C18 (24 g cartridge, 0-100% (0.1% Formic acid in MeCN)/(0.1% Formic Acid in Water)) to afford the desired product as an off white solid, to give (R)-7-(pyrrolidin-2-ylmethyl)-5H-[1,3]dioxolo[4,5-f]indole, Formate (124.5 mg, 0.39 mmol, 26%).

m/z 245.2 (M+H)⁺ (ES+)

¹H NMR (500 MHz, DMSO) δ 10.72 (s, 1H), 8.40 (s, 1H), 7.06 (d, J=2.4 Hz, 1H), 7.04 (s, 1H), 6.87 (s, 1H), 5.91 (s, 2H), 3.55 (q, J=7.5 Hz, 1H), 3.18-3.08 (m, 1H), 3.04-2.91 (m, 2H), 2.83 (dd, J=14.4, 7.5 Hz, 1H), 1.90 (tdd, J=13.3, 7.9, 5.0 Hz, 2H), 1.83-1.71 (m, 1H), 1.59-1.49 (m, 1H). Formate salt. Missing one exchangeable proton.

The material was screened by SFC (Waters UPC2, with a PDA and a QDA detector). The optimal conditions are using a Lux A1 column, 4.6×250 mm, 5 μm, with mobile phase 25% methanol (27 mM NH3), 75% CO₂. The flow rate was 4 mL/min. The run time was 5.0 mins. EE of 90.7%.

### Example 21: Synthesis of (R)-3-((1-((1H-pyrazol-5-yl)methyl)pyrrolidin-2-yl)methyl)-6-fluoro-5-methoxy-1H-indole (Compound 19B)

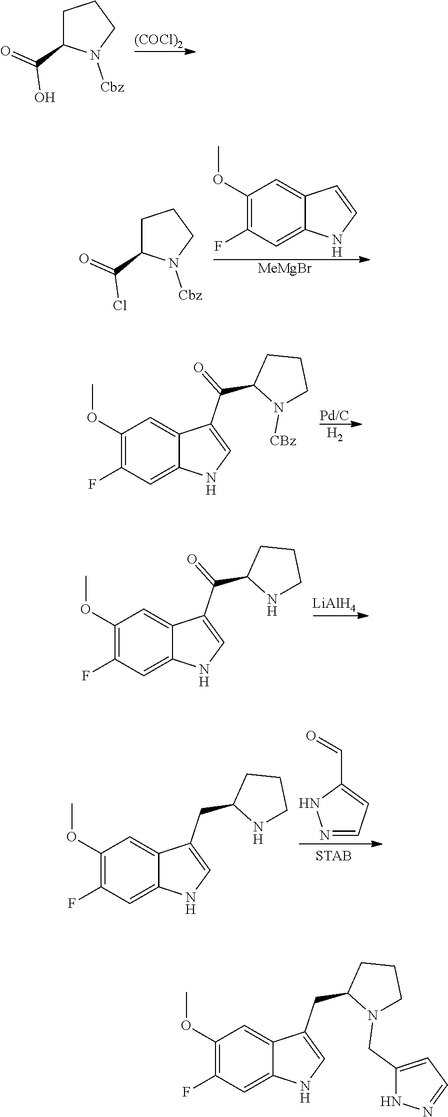

### Step 1: Benzyl (R)-2-(chlorocarbonyl)pyrrolidine-1-carboxylate

To a stirred solution of ((benzyloxy)carbonyl)-D-proline (1.32 g, 1 Eq, 5.30 mmol) in DCM (15.0 mL) under a nitrogen atmosphere at 0° C. was added oxalyl dichloride (3.33 g, 2.30 mL, 4.96 Eq, 26.3 mmol). The reaction mixture was stirred at rt for 3 h. The reaction mixture was concentrated in vacuo, and the resulting crude was suspended in iso-hexane (3×15 mL) and dried in vacuo to provide the sub-title compound (1.42 g, 5.30 mmol, 100%) as a pale-yellow oil.

m/z 264.2 (M−Cl+MeOH)⁺ (ES+)

### Step 2: Benzyl (R)-2-(6-fluoro-5-methoxy-1H-indole-3-carbonyl)pyrrolidine-1-carboxylate

To a stirred solution of 6-fluoro-5-methoxy-1H-indole (1.75 g, 2 Eq, 10.6 mmol) in dry Toluene (10.0 mL) under a nitrogen atmosphere at rt was added MeMgBr in diethyl ether (1.26 g, 3.54 mL, 3.00 molar, 2 Eq, 10.6 mmol) dropwise over 2 min. THF (4 mL) was added. The reaction mixture was stirred at rt for 4 h. A solution of product from step 1 above (1.42 g, 1 Eq, 5.30 mmol) in dry Toluene (15.0 mL) was added dropwise and the reaction mixture was stirred at rt for 72 h. The reaction mixture was quenched with sat. aq. NaHCO₃ (50 mL) and left to stir at rt for 1 h. The resulting suspension was extracted with EtOAc (3×50 mL), and the collected organic layers were dried over Na₂SO₄ and concentrated in vacuo. The crude product was purified by chromatography on silica gel (80 g cartridge, 25-100% EtOAc/iso-hexane) to afford sub-title compound (1.32 g, 3.2 mmol, 60%, 95% Purity).

m/z 397.1 (M+H)⁺ (ES+); 395.6 (M−H)⁻ (ES−)

¹H NMR (500 MHz, DMSO) δ 11.95 (s, 1H), 8.39 (d, J=10.6 Hz, 1H), 7.89-7.80 (m, 1H), 7.42-7.31 (m, 3H), 7.17-7.00 (m, 3H), 5.24-5.12 (m, 1H), 5.10-5.00 (m, 1H), 4.94 (q, J=13.2 Hz, 1H), 3.86 (s, 3H), 3.56-3.47 (m, 2H), 2.42-2.28 (m, 2H), 1.91-1.80 (m, 2H).

¹⁹F NMR (471 MHz, DMSO) δ −138.33 (m).

### Step 3: (R)-6-Fluoro-5-methoxy-3-prolyl-1H-indole

A stirred solution of the product from step 2 above (1.32 g, 1 Eq, 3.33 mmol) and Pd/C (87 L) (354 mg, 5% Wt, 0.05 Eq, 166 μmol) in MeOH (100.0 mL) under a nitrogen atmosphere at rt was charged with hydrogen (1 bar). The reaction mixture was stirred at rt for 18 h. The reaction mixture was filtered through glass microfibres, rinsing with MeOH (5 mL). The solvent was removed in vacuo to afford sub-title compound (840 mg, 3.0 mmol, 91%).

m/z 263.5 (M+H)⁺ (ES+); 261.0 (M−H)⁻ (ES−)

¹H NMR (500 MHz, DMSO) δ 8.34 (s, 1H), 7.84 (d, J=8.7 Hz, 1H), 7.33 (d, J=11.4 Hz, 1H), 4.40 (dd, J=8.7, 6.0 Hz, 1H), 3.86 (s, 3H), 3.06 (dt, J=11.6, 6.2 Hz, 1H), 2.78 (dt, J=10.4, 6.5 Hz, 1H), 2.22-2.11 (m, 1H), 1.77-1.69 (m, 1H), 1.69-1.59 (m, 2H). Two exchangeable protons not observed.

¹⁹F NMR (471 MHz, DMSO) δ −138.43.

### Step 4: (R)-6-Fluoro-5-methoxy-3-(pyrrolidin-2-ylmethyl)-1H-indole

To a stirred suspension of the product from step 3 above (835 mg, 1 Eq, 3.18 mmol) in dry THF (30.0 mL) under a nitrogen atmosphere at 0° C. was added LiAlH₄ in THF (6.50 mL, 2.00 molar, 4.08 Eq, 13.0 mmol). The reaction mixture was heated to 66° C. and stirred for 2 h. The reaction mixture was cooled to 0° C. and quenched with 3:1 THF/water (16 mL). The mixture was warmed to rt and stirred for 1 h before being filtered. Na₂SO₄ was added to the filtrate and the mixture was filtered and concentrated in vacuo to afford sub-title compound (750 mg, 1.9 mmol, 61%) as a brown gum.

m/z 249.5 (M+H)⁺ (ES+)

### Step 5: (R)-3-((1-((1H-pyrazol-5-yl)methyl)pyrrolidin-2-yl)methyl)-6-fluoro-5-methoxy-1H-indole

To a stirred solution of the product from step 4 above (85.0 mg, 1 Eq, 342 μmol) in dry THF (3.00 mL) at 22° C. was added 1H-pyrazole-5-carbaldehyde (37.0 mg, 1.12 Eq, 385 μmol). The reaction mixture was stirred for 10 min. Sodium triacetoxyhydroborate (210 mg, 2.89 Eq, 991 μmol) was added and the reaction mixture left to stir at rt for 18 h. The reaction mixture was quenched with sat. aq. NaHCO₃ (10 mL) and left to stir for 15 min. The aqueous suspension was extracted with EtOAc (10 mL), and the collected organic layers were washed with brine (10 mL), dried over Na₂SO₄, filtered and concentrated in vacuo. The crude material was dissolved in DMSO, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge BEH C18 ODB prep column, 130 Å, 5 μm, 30 mm×100 mm, flow rate 40 mL min⁻¹ eluting with a 0.3% Ammonia in water-MeCN gradient over 12.5 mins using UV across all wavelengths with PDA as well as a QDA and ELS detector. At-column dilution pump gives 2 mL min⁻¹ Methanol over the entire method, which is included in the following MeCN percentages. Gradient information: 0.0-0.5 min, 20% MeCN; 0.5-10.5 min, ramped from 20% MeCN to 50% MeCN; 10.5-10.6 min, ramped from 50% MeCN to 100% MeCN; 10.6-12.5 min, held at 100% MeCN. The clean fractions were evaporated in a Freeze-dryer. The dried fractions were reanalyzed revealing the purity of <90%. Additional screening was done using achiral SFC and suitable method was found. The impure material was dissolved in MeOH and was then separated by achiral SFC on a Waters Prep 100 with a PDA and QDa detectors, 40° C., 120 bar. The column was a Torus Diol, 19×100 mm, 5 μm, flow rate 65 mL/min eluting with a C02-MeOH gradient over 7 mins using UV across all wavelengths. Gradient information: 0.0-5.0 min, ramped from 10% MeOH to 30% MeOH; 5.0-5.1 min, ramped from 30% MeOH to 50% MeOH; 5.1-7.0 min, held at 50% MeOH. The clean fractions were pooled and concentrated to dryness using a rotary evaporator (at 30 C). The residues were re-dissolved in MeOH, transferred into final vials and evaporated on a Biotage V10 (36 C, 30 mbar) to afford title compound (30.0 mg, 87 mol, 25%).

m/z 329.2 (M+H)⁺ (ES+); 327.2 (M−H)⁻ (ES−)

¹H NMR (500 MHz, DMSO) δ 12.37 (s, 1H), 10.45 (s, 1H), 7.49 (s, 1H), 7.16-7.08 (m, 2H), 7.06 (s, 1H), 6.15 (d, J=2.0 Hz, 1H), 3.99 (d, J 13.7 Hz, 1H), 3.84 (s, 3H), 3.60 (d, J 13.8 Hz, 1H), 3.00 (ddd, J=14.3, 4.0, 1.0 Hz, 1H), 2.94 (ddd, J=8.9, 7.2, 3.1 Hz, 1H), 2.82-2.72 (m, 1H), 2.59-2.51 (m, 1H), 2.35 (td, J=8.9, 7.5 Hz, 1H), 1.78-1.44 (m, 4H).

¹⁹F NMR (471 MHz, DMSO) δ −141.25.

The product was dissolved in MeOH to 1 mg/mL and screened by SFC (Waters UPC2, with a PDA and a QDA detector). The optimal conditions are using a Lux A1 column, 4.6×250 mm, 5 μm, with mobile phase 30% Ethanol (28 mM NH3), 70% CO2. The flow rate was 4 mL/min. The run time was 4.5 min.

ee=50.82%.

### Example 22: Synthesis of (R)-3-((1-cyclopropylpyrrolidin-2-yl)methyl)-6-fluoro-5-methoxy-1H-indole, Formic Acid (Compound 16B)

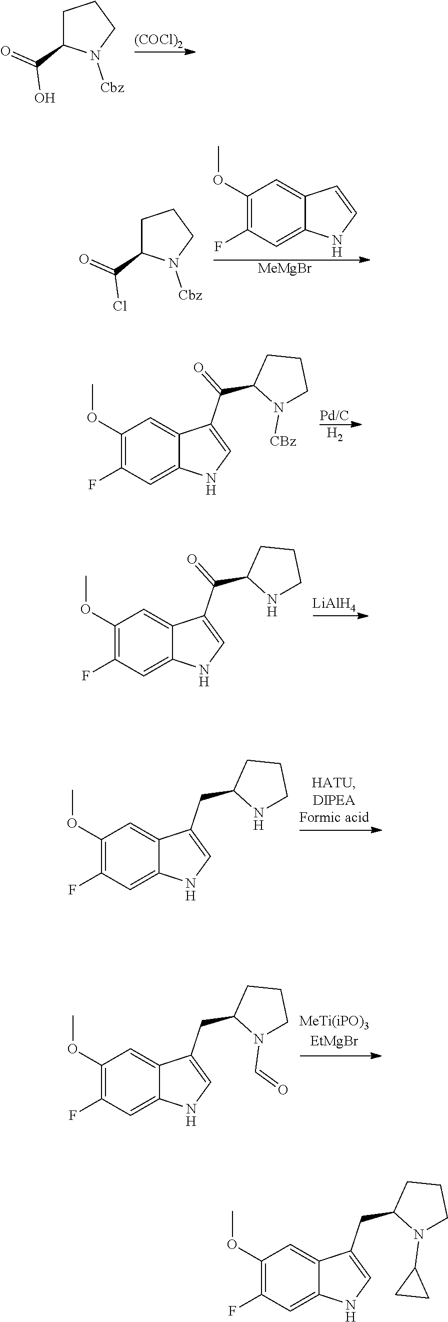

### Step 1: Benzyl (R)-2-(chlorocarbonyl)pyrrolidine-1-carboxylate

To a stirred solution of ((benzyloxy)carbonyl)-D-proline (1.32 g, 1 Eq, 5.30 mmol) in DCM (15.0 mL) under a nitrogen atmosphere at 0° C. was added oxalyl dichloride (3.33 g, 2.30 mL, 4.96 Eq, 26.3 mmol). The reaction mixture was stirred at rt for 3 h. The reaction mixture was concentrated in vacuo, and the resulting crude was suspended in iso-hexane (3×15 mL) and dried in vacuo to provide the sub-title compound (1.42 g, 5.30 mmol, 100%) as a pale-yellow oil.

m/z 264.2 (M−Cl+MeOH)⁺ (ES⁺)

### Step 2: Benzyl (R)-2-(6-fluoro-5-methoxy-1H-indole-3-carbonyl)pyrrolidine-1-carboxylate

To a stirred solution of 6-fluoro-5-methoxy-1H-indole (1.75 g, 2 Eq, 10.6 mmol) in dry Toluene (10.0 mL) under a nitrogen atmosphere at rt was added MeMgBr in diethyl ether (1.26 g, 3.54 mL, 3.00 molar, 2 Eq, 10.6 mmol) dropwise over 2 min. THF (4 mL) was added. The reaction mixture was stirred at rt for 4 h. A solution of product from step 1 above (1.42 g, 1 Eq, 5.30 mmol) in dry Toluene (15.0 mL) was added dropwise and the reaction mixture was stirred at rt for 72 h. The reaction mixture was quenched with sat. aq. NaHCO₃ (50 mL) and left to stir at rt for 1 h. The resulting suspension was extracted with EtOAc (3×50 mL), and the collected organic layers were dried over Na₂SO₄ and concentrated in vacuo. The crude product was purified by chromatography on silica gel (80 g cartridge, 25-100% EtOAc/iso-hexane) to afford sub-title compound (1.32 g, 3.2 mmol, 60%, 95% Purity).

m/z 397.1 (M+H)⁺ (ES+); 395.6 (M−H)⁻ (ES−)

¹H NMR (500 MHz, DMSO) δ 11.95 (s, 1H), 8.39 (d, J=10.6 Hz, 1H), 7.89-7.80 (m, 1H), 7.42-7.31 (m, 3H), 7.17-7.00 (m, 3H), 5.24-5.12 (m, 1H), 5.10-5.00 (m, 1H), 4.94 (q, J=13.2 Hz, 1H), 3.86 (s, 3H), 3.56-3.47 (m, 2H), 2.42-2.28 (m, 2H), 1.91-1.80 (m, 2H).

¹⁹F NMR (471 MHz, DMSO) δ −138.33 (m).

### Step 3: (R)-6-Fluoro-5-methoxy-3-prolyl-1H-indole

A stirred solution of the product from step 2 above (1.32 g, 1 Eq, 3.33 mmol) and Pd/C (87 L) (354 mg, 5% Wt, 0.05 Eq, 166 μmol) in MeOH (100.0 mL) under a nitrogen atmosphere at rt was charged with hydrogen (1 bar). The reaction mixture was stirred at rt for 18 h. The reaction mixture was filtered through glass microfibres, rinsing with MeOH (5 mL). The solvent was removed in vacuo to afford sub-title compound (840 mg, 3.0 mmol, 91%).

m/z 263.5 (M+H)⁺ (ES+); 261.0 (M−H)⁻ (ES−)

¹H NMR (500 MHz, DMSO) δ 8.34 (s, 1H), 7.84 (d, J=8.7 Hz, 1H), 7.33 (d, J=11.4 Hz, 1H), 4.40 (dd, J=8.7, 6.0 Hz, 1H), 3.86 (s, 3H), 3.06 (dt, J=11.6, 6.2 Hz, 1H), 2.78 (dt, J=10.4, 6.5 Hz, 1H), 2.22-2.11 (m, 1H), 1.77-1.69 (m, 1H), 1.69-1.59 (m, 2H). Two exchangeable protons not observed.

¹⁹F NMR (471 MHz, DMSO) δ −138.43.

### Step 4: (R)-6-Fluoro-5-methoxy-3-(pyrrolidin-2-ylmethyl)-1H-indole

To a stirred suspension of the product from step 3 above (835 mg, 1 Eq, 3.18 mmol) in dry THF (30.0 mL) under a nitrogen atmosphere at 0° C. was added LiAlH₄ in THF (6.50 mL, 2.00 molar, 4.08 Eq, 13.0 mmol). The reaction mixture was heated to 66° C. and stirred for 2 h. The reaction mixture was cooled to 0° C. and quenched with 3:1 THF/water (16 mL). The mixture was warmed to rt and stirred for 1 h before being filtered. Na₂SO₄ was added to the filtrate and the mixture was filtered and concentrated in vacuo to afford sub-title compound (750 mg, 1.9 mmol, 61%) as a brown gum.

m/z 249.5 (M+H)⁺ (ES+)

### Step 5: (R)-2-((6-fluoro-5-methoxy-1H-indol-3-yl)methyl)pyrrolidine-1-carbaldehyde

To a stirred solution of the product from step 4 above (200 mg, 70% Wt, 1 Eq, 564 μmol), formic acid (104 mg, 85.1 μL, 4 Eq, 2.26 mmol) and DIPEA (292 mg, 393 μL, 4 Eq, 2.26 mmol) in dry DMF (1.50 mL) at 22° C. was added HATU (429 mg, 2 Eq, 1.13 mmol). The reaction mixture was stirred for 18 hours. The reaction mixture was diluted with EtOAc (10 mL) and partitioned with sat. aq. sodium bicarbonate (20 mL). The aqueous layer was extracted with EtOAc (3×10 mL), and the collected organic layers washed with 1:1 Water:brine (3×20 mL), dried (Na₂SO₄), filtered and concentrated in vacuo. The crude product was purified by chromatography on silica gel (12 g cartridge, 50-100% EtOAc/isohexane) to afford the sub-title compound (28.0 mg, 94 μmol, 17%).

m/z 277.592 (M+H)⁺ (ES+)

### Step 6: (R)-3-((1-cyclopropylpyrrolidin-2-yl)methyl)-6-fluoro-5-methoxy-1H-indole, Formic Acid

To a stirred solution the product from step 5 above (28.0 mg, 93% Wt, 1 Eq, 94.2 μmol) and triisopropoxy(methyl)titanium (27.2 mg, 113 μL, 1.00 molar, 1.2 Eq, 113 μmol) in dry Tetrahydrofuran (2.50 mL) under a nitrogen atmosphere at 22° C. was added EtMgBr in THF (18.8 mg, 141 μL, 1.00 molar, 1.5 Eq, 141 μmol). The reaction mixture was stirred for 1 hours after which triisopropoxy(methyl)titanium (27.2 mg, 113 μL, 1.00 molar, 1.2 Eq, 113 μmol) and EtMgBr in THF (18.8 mg, 141 μL, 1.00 molar, 1.5 Eq, 141 μmol) were added. The reaction mixture was stirred for 2 hours. The reaction mixture was quenched with sat. aq. sodium bicarbonate (7 mL) and left to stir for 1 h. The resulting white precipitate was removed via filtration and washed with EtOAc (3×10 mL). The filtrate was partitioned and the aqueous layer washed with EtOAc (3×15 mL). The collected organic layers were dried (Na₂SO₄), filtered and concentrated in vacuo. The crude product was purified by chromatography on RP Flash C18 (12 g cartridge, 0-50% (0.1% Formic acid in MeCN)/(0.1% Formic Acid in Water)) to afford the title compound (7.00 mg, 19 mol, 20%) as an orange gum.

m/z 289.200 (M+H)⁺ (ES+)

¹H NMR (500 MHz, DMSO) δ 10.67 (s, 1H), 8.22 (s, 1H), 7.15-7.11 (m, 2H), 7.07 (d, J=2.3 Hz, 1H), 3.83 (d, J=2.7 Hz, 3H), 3.23-3.15 (m, 1H), 3.00 (ddd, J=9.5, 6.7, 3.5 Hz, 1H), 2.83 (ddt, J=10.7, 7.3, 3.6 Hz, 1H), 2.48-2.40 (m, 2H), 1.77 (tt, J=6.8, 3.7 Hz, 1H), 1.72-1.48 (m, 3H), 1.48-1.37 (m, 1H), 0.56 (dddd, J=16.1, 10.1, 7.1, 4.2 Hz, 2H), 0.45-0.36 (m, 1H), 0.29 (ddt, J=10.2, 6.5, 4.0 Hz, 1H). +1 exchangeable proton not observed.

¹⁹F NMR (471 MHz, DMSO) δ −141.47.

### Example 23: Synthesis of (R)-6-chloro-5-methoxy-3-((1-methylpyrrolidin-2-yl)methyl)-1H-indole, formate (Compound 24B)

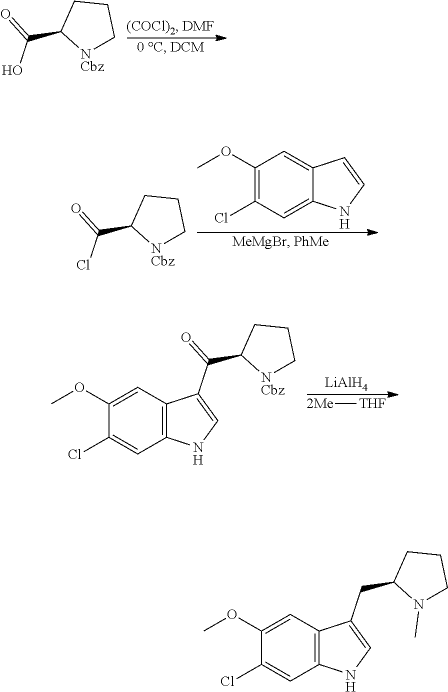

### Step 1: Benzyl (R)-2-(chlorocarbonyl)pyrrolidine-1-carboxylate

To a solution of ((benzyloxy)carbonyl)-D-proline (690 mg, 1 Eq, 2.77 mmol) in dry Dichloromethane (10.0 mL) at 0° C. under a nitrogen atmosphere was added oxalyl chloride (1.74 g, 1.20 mL, 4.95 Eq, 13.7 mmol) and 2 drops of N,N-dimethylformamide (20.2 mg, 21.4 μL, 0.1 Eq, 277 μmol). The reaction mixture was left to stir at 22° C. for 1 hour. The reaction mixture was concentrated in vacuo resuspended in isohexane (3×10 mL) and dried in vacuo to afford benzyl (R)-2-(chlorocarbonyl)pyrrolidine-1-carboxylate (741 mg, 2.7 mmol, 99%) as a dark yellow oil.

Product used without analysis.

### Step 2: Benzyl (R)-2-(6-chloro-5-methoxy-1H-indole-3-carbonyl)pyrrolidine-1-carboxylate

To a solution of 6-chloro-5-methoxy-1H-indole (900 mg, 2 Eq, 4.96 mmol) in dry Toluene (15.0 mL) under a nitrogen atmosphere at 22° C. was added methylmagnesium bromide in diethyl ether (591 mg, 1.65 mL, 3.00 molar, 2 Eq, 4.96 mmol). The reaction mixture was stirred for 2 hours. A solution of benzyl (R)-2-(chlorocarbonyl)pyrrolidine-1-carboxylate (663 mg, 1 Eq, 2.48 mmol) in dry Toluene (15.0 mL) was added dropwise and the reaction mixture was left to stir for 18 hours. The reaction mixture was quenched with water (100 mL) and the resulting suspension was extracted with EtOAc (3×100 mL). The collected organic layers were dried over magnesium sulfate and concentrated in vacuo. The resulting crude material was triturated with EtOAc/hexane and dried in vacuo to afford benzyl (R)-2-(6-chloro-5-methoxy-1H-indole-3-carbonyl)pyrrolidine-1-carboxylate (587 mg, 1.2 mmol, 48%).

m/z 413.3 (M+H)⁺ (ES+)

1H NMR (500 MHz, CDCl3) δ 10.24 (s, 1H), 7.51 (d, J=3.2 Hz, 1H), 7.47-7.39 (m, 4H), 7.39-7.31 (m, 1H), 7.22 (s, 1H), 7.03 (s, 1H), 5.31-5.19 (m, 2H), 5.03 (dd, J=8.9, 2.9 Hz, 1H), 3.87-3.77 (m, 4H), 3.68-3.58 (m, 1H), 2.31-2.20 (m, 1H), 2.10-1.87 (m, 3H).

### Step 3: (R)-6-chloro-5-methoxy-3-((1-methylpyrrolidin-2-yl)methyl)-1H-indole, Formate

To a stirred solution of benzyl (R)-2-(6-chloro-5-methoxy-1H-indole-3-carbonyl)pyrrolidine-1-carboxylate (200 mg, 1 Eq, 484 μmol) in dry 2-Methyltetrahydrofuran (4.00 mL) under a nitrogen atmosphere at 0° C. was added LiAlH₄ in THF (73.5 mg, 969 μL, 2.00 molar, 4 Eq, 1.94 mmol). The reaction mixture was heated to 66° C. for 4 hours. 1 The heating was stopped and the reaction stirred at room temperature overnight. The reaction mixture was cooled to 0° C. and quenched using a 1:1 mixture of Glauber's salt (Na₂SO₄·10H₂O) and Celite added portion wise. The mixture was then filtered and the solid washed with TBME (2×20 ml). The filtrate was dried over MgSO₄ and concentrated in vacuo to give a clear viscous liquid. The crude product was purified by chromatography on RP Flash C18 (24 g cartridge, 0-100% (0.1% Formic acid in MeCN)/(0.1% Formic Acid in Water)) to afford the desired product as a colourless sticky gum. Material was dissolved in the minimum amount of water (10% MeCN). The material was then freeze dried overnight to give (R)-6-chloro-5-methoxy-3-((1-methylpyrrolidin-2-yl)methyl)-1H-indole, Formate (87.9 mg, 0.27 mmol, 55%) as a colourless gummy foam.

m/z 278.9 (M+H)⁺ (ES+)

1H NMR (500 MHz, DMSO) δ 10.76 (s, 1H), 8.21 (s, 1H), 7.37 (s, 1H), 7.16 (s, 2H), 3.84 (s, 3H), 3.07 (s, 2H), 2.56 (d, J=9.5 Hz, 2H), 2.42 (s, 3H), 2.27 (q, J=8.8 Hz, 1H), 1.76 (dt, J=15.5, 5.9 Hz, 1H), 1.62 (t, J=8.2 Hz, 2H), 1.54-1.44 (m, 1H).

The product was screened by SFC (Waters UPC2, with a PDA and a QDA detector). The optimal conditions are using a Lux iC5 column, 4.6×250 mm, 5 μm, with mobile phase 35% methanol (27 mM NH₃), 65% CO₂. The flow rate was 4 mL/min. The run time was 4.5 mins. ee of >99.9%.

### Example 24: Synthesis of (R)-6-chloro-5-methoxy-3-(pyrrolidin-2-ylmethyl)-1H-indole, Formic Acid & (R)-3-((1-benzylpyrrolidin-2-yl)methyl)-6-chloro-5-methoxy-1H-indole (Compound 22B)

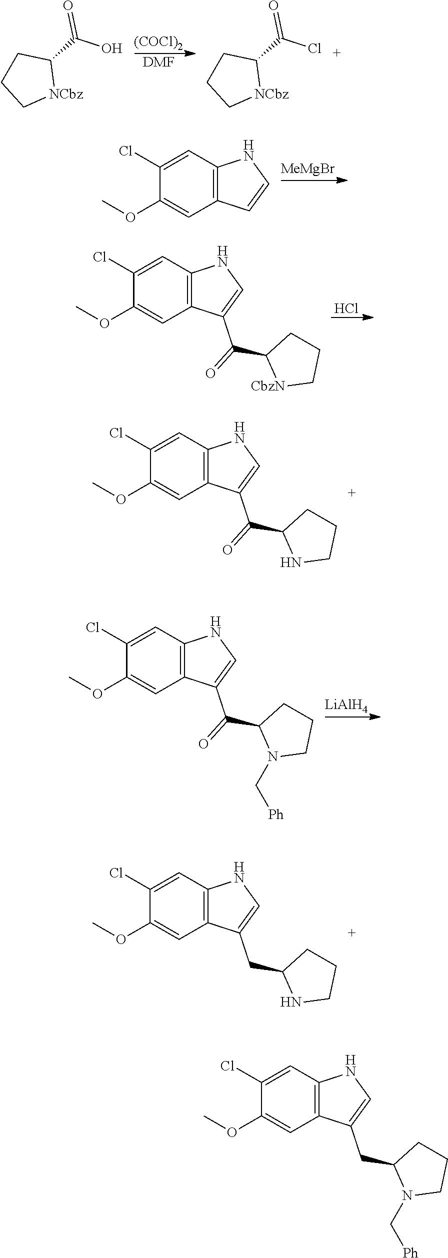

### Step 1: Benzyl (R)-2-(chlorocarbonyl)pyrrolidine-1-carboxylate

To a solution of ((benzyloxy)carbonyl)-D-proline (690 mg, 2.77 mmol) in dry CH₂Cl₂ (10.0 mL) at 0° C. under a nitrogen atmosphere was added oxalyl chloride (1.20 mL, 13.7 mmol) and 2 drops of DMF (21.0 μL, 277 μmol) dropwise. The reaction mixture was left to stir at 22° C. for 1 hour. The reaction mixture was concentrated in vacuo resuspended in isohexane (3×10 mL) and dried in vacuo to afford benzyl (R)-2-(chlorocarbonyl)pyrrolidine-1-carboxylate (741 mg, 2.7 mmol, 99%) as a dark yellow oil.

The product was used without analysis.

### Step 2: Benzyl (R)-2-(6-chloro-5-methoxy-1H-indole-3-carbonyl)pyrrolidine-1-carboxylate

To a solution of 6-chloro-5-methoxy-1H-indole (900 mg, 4.96 mmol) in dry PhMe (15.0 mL) under a nitrogen atmosphere at 22° C. was added MeMgBr in Et₂O (1.65 mL, 3.00 molar, 4.96 mmol). The reaction mixture was stirred for 2 hours. A solution of benzyl (R)-2-(chlorocarbonyl)pyrrolidine-1-carboxylate (663 mg, 2.48 mmol) in dry PhMe (15.0 mL) was added dropwise and the reaction mixture was left to stir for 18 hours. The reaction mixture was quenched with water (100 mL) and the resulting suspension was extracted with EtOAc (3×100 mL). The collected organic layers were dried over magnesium sulfate and concentrated in vacuo. The resulting crude material was triturated with EtOAc/hexane and dried in vacuo to afford benzyl (R)-2-(6-chloro-5-methoxy-1H-indole-3-carbonyl)pyrrolidine-1-carboxylate (587 mg, 1.2 mmol, 48%).

m/z 413.3 (M+H)⁺ (ES+)

¹H NMR (500 MHz, CDCl3) δ 10.24 (s, 1H), 7.51 (d, J=3.2 Hz, 1H), 7.47-7.39 (m, 4H), 7.39-7.31 (m, 1H), 7.22 (s, 1H), 7.03 (s, 1H), 5.31-5.19 (m, 2H), 5.03 (dd, J=8.9, 2.9 Hz, 1H), 3.87-3.77 (m, 4H), 3.68-3.58 (m, 1H), 2.31-2.20 (m, 1H), 2.10-1.87 (m, 3H).

### Step 3: (R)-6-chloro-5-methoxy-3-prolyl-1H-indole & 3-(benzyl-D-prolyl)-6-chloro-5-methoxy-1H-indole

To a solution of benzyl (R)-2-(6-chloro-5-methoxy-1H-indole-3-carbonyl)pyrrolidine-1-carboxylate (172 mg, 417 μmol) in 2-MeTHF (2.50 mL) at 22° C. was added HCl in iPrOH (2.50 mL, 5.00 molar, 12.5 mmol). The reaction mixture was left to stir for 2 hours. The reaction mixture was heated to 75° C. and left to stir for 2 days. The reaction mixture was diluted with water (10 mL) quenched with sat. sodium bicarbonate (40 mL) and extracted with 3:1 CHCl₃/iPrOH (3×40 mL). The collected organic layers were dried over magnesium sulfate and concentrated in vacuo to afford (R)-6-chloro-5-methoxy-3-prolyl-1H-indole and 3-(benzyl-D-prolyl)-6-chloro-5-methoxy-1H-indole (116 mg, 0.23 mmol, 56%).

m/z 279.2 (M+H)⁺ (ES+)

### Step 4: (R)-6-chloro-5-methoxy-3-(pyrrolidin-2-ylmethyl)-1H-indole, Formic Acid & (R)-3-((1-benzylpyrrolidin-2-yl)methyl)-6-chloro-5-methoxy-1H-indole

To a mixture of (R)-6-chloro-5-methoxy-3-prolyl-1H-indole and 3-(benzyl-D-prolyl)-6-chloro-5-methoxy-1H-indole (116 mg, 233 μmol) in dry 2-MeTHF (2.00 mL) under a nitrogen atmosphere at 0° C. was added LiAlH₄ in THF (1.00 mL, 2.00 molar, 2.00 mmol) dropwise. The reaction mixture was heated to 65° C. and left to stir for 2 hours. The reaction mixture was cooled to 0° C. and quenched with sodium sulfate decahydrate. The resulting solids were removed via filtration and washed with EtOAc (3×20 mL). The collected filtrate was dried over magnesium sulfate and concentrated in vacuo.

The crude product was purified by chromatography on RP Flash C18 (24 g cartridge, 0-100% (0.1% Formic acid in MeCN)/(0.1% Formic Acid in Water)) to afford (R)-6-chloro-5-methoxy-3-(pyrrolidin-2-ylmethyl)-1H-indole, Formic Acid (37.0 mg, 0.12 mmol, 50%, 97% Purity).

m/z 265.2 (M+H)⁺ (ES+)

¹H NMR (400 MHz, DMSO) δ 10.90 (s, 1H), 8.39 (s, 1H), 7.40 (s, 1H), 7.27 (s, 2H), 3.86 (s, 3H), 3.66 (s, 1H), 3.23-2.90 (m, 4H), 2.02-1.74 (m, 3H), 1.61-1.57 (m, 1H).

2 protons not observed due to exchange.

The remaining crude material was purified by chromatography on silica gel (24 g cartridge, 0-100% (2% NH₄OH in 3:1 EtOAc/EtOH) in isohexane) to afford (R)-3-((1-benzylpyrrolidin-2-yl)methyl)-6-chloro-5-methoxy-1H-indole (6.00 mg, 15 mol, 6.5%, 90% Purity) as a pale brown gum.

m/z 355.3 (M+H)⁺ (ES+)

¹H NMR (500 MHz, DMSO) δ 10.73 (s, 1H), 7.42-7.29 (m, 5H), 7.26-7.21 (m, 1H), 7.16 (d, J=2.3 Hz, 1H), 7.12 (s, 1H), 4.14 (d, J=13.1 Hz, 1H), 3.81 (s, 3H), 3.31 (d, J=13.2 Hz, 1H), 3.04 (dd, J=14.2, 3.8 Hz, 1H), 2.82 (ddd, J=9.7, 6.7, 3.4 Hz, 1H), 2.79-2.71 (m, 1H), 2.60 (dd, J=14.2, 8.9 Hz, 1H), 2.15 (dd, J=8.6, 8.5 Hz, 1H), 1.79-1.68 (m, 1H), 1.65-1.45 (m, 3H).

### Example 25: (R)-5,6-difluoro-3-((1-((3-methoxypyridin-4-yl)methyl)pyrrolidin-2-yl)methyl)-1H-indole (Compound 30A)

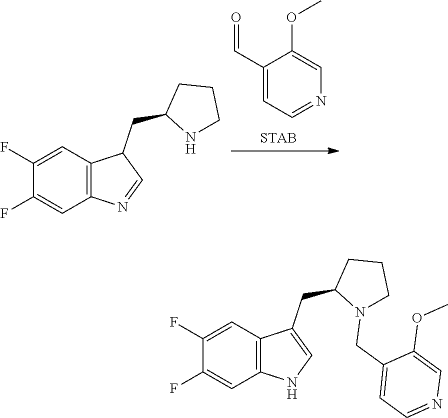

### Step 1: (R)-5,6-difluoro-3-((1-((3-methoxypyridin-4-yl)methyl)pyrrolidin-2-yl)methyl)-1H-indole (4235-93/4144-198)

To a stirred solution of (R)-5,6-difluoro-3-(pyrrolidin-2-ylmethyl)-1H-indole (see CMP-00001270) (100 mg, 1 Eq, 254 μmol) in dry THF (3.00 mL) at 22° C. was added 3-methoxyisonicotinaldehyde (52.2 mg, 1.5 Eq, 381 μmol). The reaction mixture was stirred for 35 min. Sodium triacetoxyhydroborate (161 mg, 3 Eq, 762 μmol) was added and the reaction mixture left to stir for 17 h at rt. The reaction mixture was quenched with sat. aq. NaHCO₃ (5 mL) and left to stir for 30 min. The aq. suspension was diluted with water (5 mL) and extracted with EtOAc (3×10 mL). The combined organic layers were dried over Na₂SO₄, filtered and concentrated in vacuo. The crude product was purified by chromatography on silica gel (4 g Redisep Gold cartridge, 0-50% (2% NH₄OH in 3:1 EtOAc/EtOH) in iso-hexane) to afford the product as a yellow solid. The solid was triturated from MTBE (2 mL), filtered and dried in a vacuum desiccator at 45° C. for 3 days to afford title compound (12.0 mg, 32 mol, 13%) as a white solid.

m/z 358.2 (M+H)⁺ (ES+); 356.2 (M−H)⁻ (ES−)

¹H NMR (500 MHz, DMSO) δ 10.94 (s, 1H), 8.30 (s, 1H), 8.18 (d, J=4.6 Hz, 1H), 7.46 (dd, J=11.5, 8.0 Hz, 1H), 7.39 (d, J=4.7 Hz, 1H), 7.30 (dd, J=11.3, 7.0 Hz, 1H), 7.22 (d, J=2.3 Hz, 1H), 4.01 (d, J 15.1 Hz, 1H), 3.90 (s, 3H), 3.48 (d, J 15.2 Hz, 1H), 3.02-2.93 (m, 2H), 2.82-2.73 (m, 1H), 2.57 (dd, J=14.2, 8.7 Hz, 1H), 2.14 (q, J=8.5 Hz, 1H), 1.78-1.67 (m, 1H), 1.63-1.56 (m, 2H), 1.54-1.47 (m, 1H).

¹⁹F NMR (471 MHz, DMSO) δ −146.31 (d, J=22.1 Hz), −149.65 (d, J=22.1 Hz).

The product was dissolved in MeOH to 1 mg/mL and screened by SFC (Waters UPC2, with a PDA and a QDA detector). The optimal conditions are using a Chiralpak IG column, 4.6×250 mm, 5 μm, with mobile phase 25% Methanol (28 mM NH3), 75% CO2. The flow rate was 4 mL/min. The run time was 3.5 min.

ee=38.30%

### Example 26: (R)-5,6-difluoro-3-((1-((1-methyl-1H-pyrazol-4-yl)methyl)pyrrolidin-2-yl)methyl)-1H-indole (Compound 31A)

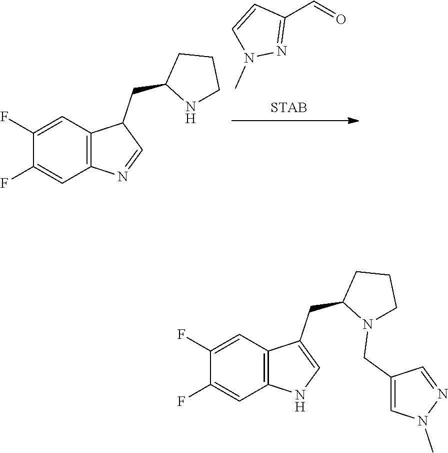

### Step 1: (R)-5,6-difluoro-3-((1-((1-methyl-1H-pyrazol-4-yl)methyl)pyrrolidin-2-yl)methyl)-1H-indole (4235-92/4144-197)

To a stirred suspension of (R)-5,6-difluoro-3-(pyrrolidin-2-ylmethyl)-1H-indole (see CMP-00001270) (100 mg, 60% Wt, 1 Eq, 254 μmol) in dry Tetrahydrofuran (3.00 mL) at 22° C. was added 1-methyl-1H-pyrazole-3-carbaldehyde (44.2 mg, 1.5 Eq, 381 μmol). The reaction mixture was stirred for 35 min. Sodium triacetoxyhydroborate (161 mg, 3 Eq, 762 μmol) was added and the reaction mixture left to stir for 17 h at rt. The reaction mixture was quenched with sat. aq. NaHCO₃ (5 mL) and left to stir for 30 min. The aq. suspension was diluted with water (5 mL) and extracted with EtOAc (3×10 mL). The combined organic layers were dried over Na₂SO₄, filtered and concentrated in vacuo. The crude product was purified by chromatography on silica gel (4 g Redisep Gold cartridge, 0-50%, (2% NH₄OH in 3:1 EtOAc/EtOH) in iso-hexane) to afford the product as a yellow gum. The crude product was purified by chromatography on silica gel (12 g cartridge, 20-60% MeCN/(0.1% Ammonium Hydroxide in Water)) to afford title compound (4.00 mg, 12 mol, 4.5%) as a white solid.

m/z 331.2 (M+H)⁺ (ES+); 329.2 (M−H)⁻ (ES−)

¹H NMR (500 MHz, DMSO) δ 10.91 (s, 1H), 7.60 (d, J=2.1 Hz, 1H), 7.52 (dd, J=11.6, 8.1 Hz, 1H), 7.29 (dd, J=11.3, 7.0 Hz, 1H), 7.20 (d, J=2.3 Hz, 1H), 6.13 (d, J=2.2 Hz, 1H), 3.90 (d, J=13.6 Hz, 1H), 3.82 (s, 3H), 3.54 (d, J=13.6 Hz, 1H), 3.01 (dd, J=14.0, 3.5 Hz, 1H), 2.89 (t, J=8.2 Hz, 1H), 2.68-2.63 (m, 1H), 2.45 (dd, J=13.9, 9.3 Hz, 1H), 2.29 (q, J=8.4 Hz, 1H), 1.66-1.36 (m, 4H).

¹⁹F NMR (471 MHz, DMSO) δ −146.35 (d, J=22.1 Hz), −149.76 (d, J=22.1 Hz).

The product (in 0.4 mL DMSO) was further diluted with MeOH to 1 mg/mL and screened by SFC (Waters UPC2, with a PDA and a QDA detector). The optimal conditions are using a Lux C3 column, 4.6×250 mm, 5 μm, with mobile phase 15% Methanol (28 mM NH₃), 85% CO2. The flow rate was 4 mL/min. The run time was 2.5 mins.

ee=40.26%

### Example 27: Functional Assays at 5-HT_{2A}, 5-HT_{2C}, and 5-HT_{1A} Receptors

Agonist activity at 5-HT_{2A}, 5-HT_{2C}, and 5-HT_{1A} receptors was assessed using Revvity's HTRF assay kits (IP-One kit for 5-HT_{2A} and 5-HT_{2C}; cAMP kit for 5-HT_{1A}). Test compounds, dissolved in DMSO, were dispensed into 384-well plates using an ECHO acoustic dispenser to generate a ten-point half-log concentration-response curve. CHO-K1 cells stably expressing the respective serotonin receptors (ValiScreen GPCR Cell Lines ES-315-C and ES-313-C from PerkinElmer, or cAMP Hunter 95-0132C₂ from DiscoverX) were added using a Multidrop dispenser and incubated at 37° C. with 5% CO₂.

For assays using 5-HT_{1A}-expressing cells, test compounds were co-incubated with 2 μM forskolin to elevate basal cAMP levels and 10 nM SB 224289 to block endogenous 5-HT_{1B} receptor activity.

Following incubation, cells were lysed by adding the kit's lysis buffer supplemented with donor (cryptate-conjugated) and acceptor (d2-labeled) antibodies specific for IP₁ (for 5-HT_{2A} and 5-HT_{2C}) or cAMP (for 5-HT_{1A}). After room-temperature incubation to allow antibody binding and HTRF signal stabilization, fluorescence emissions at 620 nm and 665 nm were measured using a PHERAstar plate reader.

A standard curve for IP₁ or cAMP was prepared in parallel and treated with the same detection reagents. The HTRF signal (665/620 nm ratio) was used to interpolate analyte concentrations from the standard curve, enabling the calculation of compound potency (pEC₅₀). The results are shown in Table 1.

TABLE 1
Assay Results

| Compound | | | | |
|---|---|---|---|---|
| No. | Structure | 2A pEC50 | 1A pEC50 | 2C pEC50 |
| 15A | 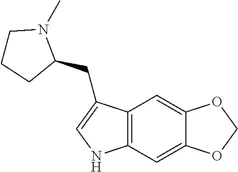 [SMILES: CN1CCC[C@@H]1Cc1c[nH]c2cc3c(cc12)OCO3] | 5.89 | 6.898 | 7.017 |
| 16B | 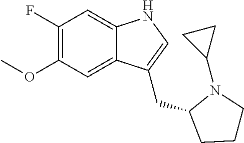 [SMILES: COc1cc2c(C[C@H]3CCCN3C3CC3)c[nH]c2cc1F] | 8.295 | 6.477 | 8.051 |
| 23A | 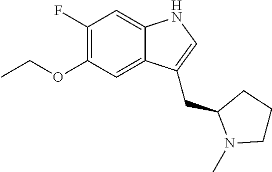 [SMILES: CCOc1cc2c(C[C@H]3CCCN3C)c[nH]c2cc1F] | 7.371 | 6.419 | — |
| 15A | 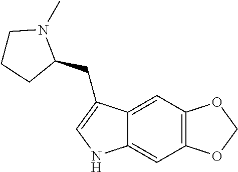 [SMILES: CN1CCC[C@@H]1Cc1c[nH]c2cc3c(cc12)OCO3] | 5.099 | 6.123 | 5.648 |
| 18A | 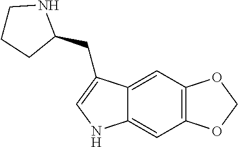 [SMILES: c1[nH]c2cc3c(cc2c1C[C@H]1CCCN1)OCO3] | 6.021 | 6.116 | 7.24 |
| 24A | 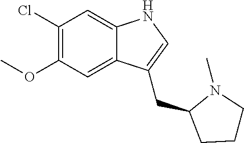 [SMILES: COc1cc2c(C[C@@H]3CCCN3C)c[nH]c2cc1Cl] | 7.372 | 5.924 | 8.657 |
| 19B | 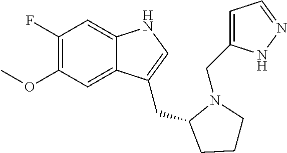 [SMILES: COc1cc2c(C[C@H]3CCCN3Cc3ccn[nH]3)c[nH]c2cc1F] | 7.511 | 5.887 | 7.784 |
| 20A | 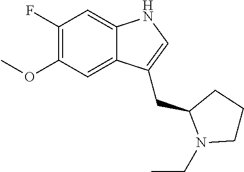 [SMILES: CCN1CCC[C@@H]1Cc1c[nH]c2cc(F)c(OC)cc12] | 7.649 | 5.837 | 7.427 |
| 21B | 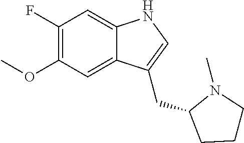 [SMILES: COc1cc2c(C[C@H]3CCCN3C)c[nH]c2cc1F] | 7.874 | 5.798 | 8.245 |
| 1B | 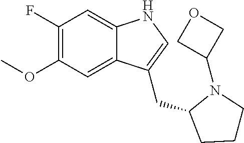 [SMILES: COc1cc2c(C[C@H]3CCCN3C3COC3)c[nH]c2cc1F] | 7.043 | 5.779 | 6.885 |
| 2B | 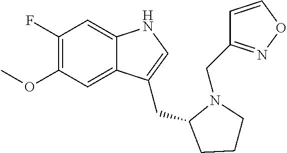 [SMILES: COc1cc2c(C[C@H]3CCCN3Cc3ccon3)c[nH]c2cc1F] | 7.505 | 5.669 | 7.272 |
| 3A | 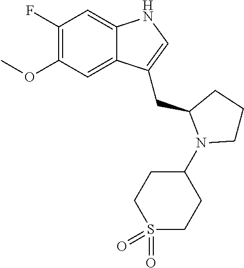 [SMILES: COc1cc2c(C[C@H]3CCCN3C3CCS(=O)(=O)CC3)c[nH]c2cc1F] | 6.558 | 5.615 | 6.627 |
| 4A | 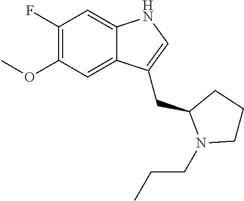 [SMILES: CCCN1CCC[C@@H]1Cc1c[nH]c2cc(F)c(OC)cc12] | 7.494 | 5.248 | 7.175 |
| 5A | 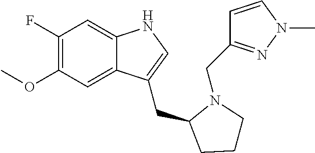 | 7.132 | 5.152 | 7.257 |
| 6B | 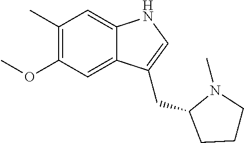 [SMILES: COc1cc2c(C[C@H]3CCCN3C)c[nH]c2cc1C] | 6.737 | 5.124 | 7.585 |
| 13C | 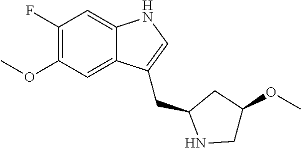 | 7.421 | 5.064 | 7.962 |
| 22B | 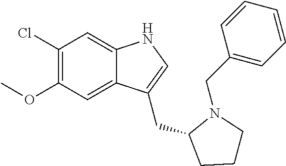 [SMILES: COc1cc2c(C[C@H]3CCCN3Cc3ccccc3)c[nH]c2cc1Cl] | −0.158 | 5 | — |
| 25A | 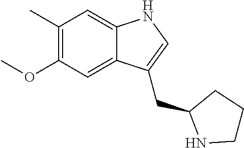 [SMILES: COc1cc2c(C[C@H]3CCCN3)c[nH]c2cc1C] | 7.23 | 5 | 7.948 |
| 26B | 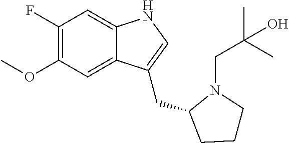 [SMILES: COc1cc2c(C[C@H]3CCCN3CC(C)(C)O)c[nH]c2cc1F] | 5.998 | 5 | 5.549 |
| 27B | 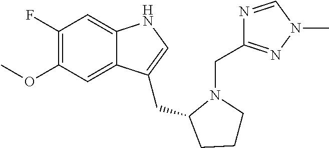 | 6.854 | 4.981 | 7.344 |
| 4A | 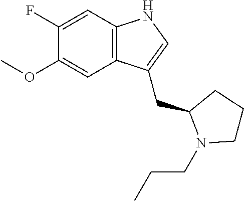 [SMILES: CCCN1CCC[C@@H]1Cc1c[nH]c2cc(F)c(OC)cc12] | 7.692 | 4.896 | 7.157 |
| 7A | 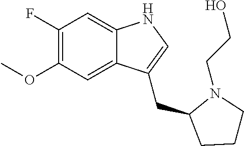 [SMILES: COc1cc2c(C[C@@H]3CCCN3CCO)c[nH]c2cc1F] | 7.445 | 4.855 | 7.552 |
| 8B | 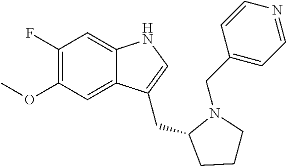 [SMILES: COc1cc2c(C[C@H]3CCCN3Cc3ccncc3)c[nH]c2cc1F] | 7.457 | 4.806 | 6.989 |
| 9B | 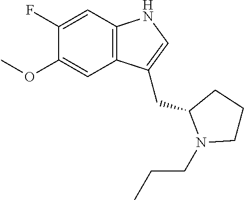 [SMILES: CCCN1CCC[C@H]1Cc1c[nH]c2cc(F)c(OC)cc12] | 5.836 | 4.787 | 5.609 |
| 10A | 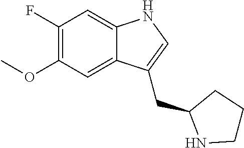 [SMILES: COc1cc2c(C[C@H]3CCCN3)c[nH]c2cc1F] | 7.697 | 4.764 | 7.647 |
| 11A | 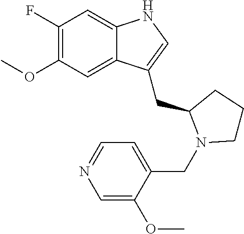 [SMILES: COc1cc2c(C[C@H]3CCCN3Cc3ccncc3OC)c[nH]c2cc1F] | 8.272 | 4.664 | 6.922 |
| 12C | 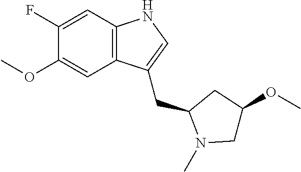 | 7.216 | 4.411 | 7.699 |
| 30A | 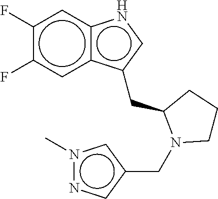 | 7.318 | 5.408 | — |
| 31A | 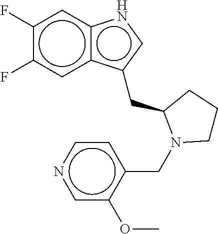 | 7.433 | 5 | 6.655 |

The publications discussed herein are provided solely for their disclosure prior to the filing date of the present application. Nothing herein is to be construed as an admission that the present invention is not entitled to antedate such publication by virtue of prior invention.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A compound of Formula (I): 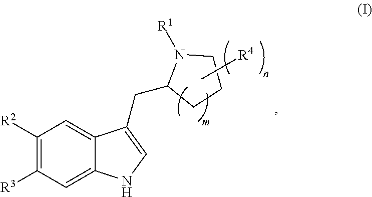
or a pharmaceutically acceptable salt, deuterated form or stereoisomer thereof
wherein:
R¹ is —H, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl; wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more -halo, oxo, —OH, —NH₂, —CN, —O—C₁₋₆ alkyl, or —C₁₋₆ alkyl;
R² is -halo, —O—C₁₋₆ alkyl, —S—C₁₋₆ alkyl, —S(O)₂—C₁₋₆ alkyl, —SH, —NH—C₁₋₆ alkyl, —N(C₁₋₆ alkyl)₂, —NH₂, —CN, —O(C═O)—C₁₋₆ alkyl, or —NH(C═O)—C₁₋₆ alkyl;
R³ is -halo, —O—C₁₋₆ alkyl, —OH, —S—C₁₋₆ alkyl, —S(O)₂—C₁₋₆ alkyl, —SH, —NH—C₁₋₆ alkyl, —N(C₁₋₆ alkyl)₂, —NH₂, —CN, —O(C═O)—C₁₋₆ alkyl, or —NH(C═O)—C₁₋₆ alkyl; or
R² and R³ together with the carbons to which they are attached form a heterocyclyl;
R⁴ is —C₁₋₆ alkyl, -aryl, -heteroaryl, —C₁₋₆ alkylene-aryl, —C₁₋₆ alkylene-heteroaryl, —C₁₋₆ alkylene-OH, —C₁₋₆ alkylene-O—C₁₋₆ alkyl, or —O—C₁₋₆ alkyl;
n is 0, 1, 2, or 3; and
m is 0 or 1,
and wherein, the compound of Formula (I) is not:

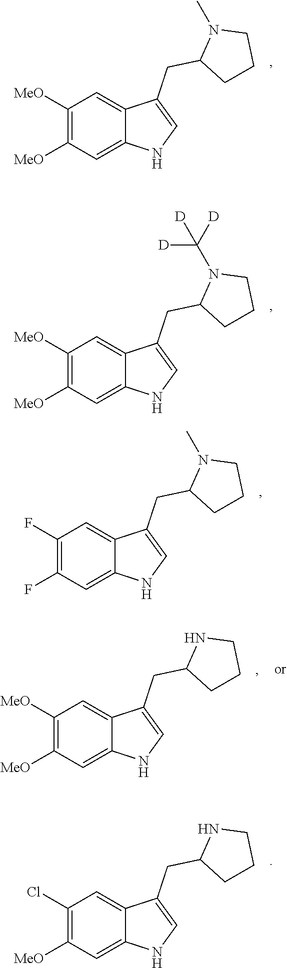

2. The compound of claim 1, wherein R² and R³ are not the same.

3. The compound of claim 1, wherein R¹ is —H, C₁₋₆ alkyl, C₁₋₆ alkylene-aryl, C₁₋₆ alkylene-heteroaryl, heterocyclyl, cycloalkyl, or C₁₋₆ alkylene-OH.

4. The compound of claim 3, wherein R¹ is C₁₋₆ alkylene-heteroaryl.

5. The compound of claim 4, wherein R¹ is

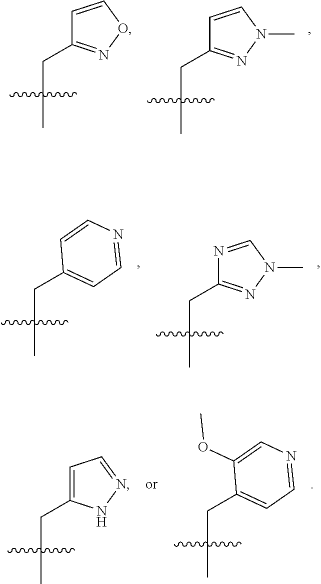

6. The compound of claim 3, wherein R¹ is an alkylene-aryl.

7. The compound of claim 6, wherein R¹ is

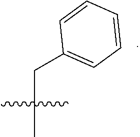 [SMILES: *Cc1ccccc1]

8. The compound of claim 3, wherein R¹ is a heterocyclyl, optionally substituted with one or more oxo.

9. The compound of claim 8, wherein R¹ is

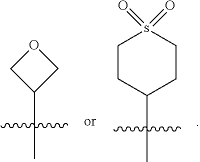

10. The compound of claim 1, wherein R² is —O—C₁₋₆ alkyl.

11. The compound of claim 1, wherein R³ is -halo or —O—C₁₋₆ alky.

12. The compound of claim 1, wherein R² and R³ together with the carbons to which they are attached from a heterocyclyl.

13. The compound of claim 12, wherein the heterocyclyl is

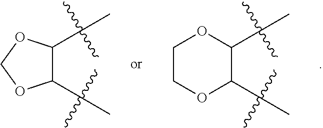 [SMILES: *C1OCOC1* ; *C1OCCOC1*]

14. The compound of claim 13, wherein the heterocyclyl is

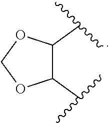 [SMILES: *C1OCOC1*]

15. The compound of claim 1, wherein n is 0.

16. The compound of claim 1, wherein n is 1.

17. The compound of claim 1, wherein m is 0.

18. The compound of claim 1, wherein m is 1.

19. The compound of claim 16, wherein R⁴ is —O—C₁₋₆ alkyl.

20. The compound of claim 1, wherein
R¹ is —H, —C₁₋₆ alkyl, aryl, —C₁₋₆ alkylene-heteroaryl, -heterocyclyl, or -cycloalkyl;
R² is —O—C₁₋₆ alkyl or —S—C₁₋₆ alkyl; and
R³ is -halo or —O—C₁₋₆ alkyl.

21. The compound of claim 1, wherein
R¹ is —H or —C₁₋₆ alkyl; and
R² and R³ together with the carbon to which they are attached form

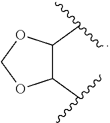 [SMILES: *C1OCOC1*]

22. The compound of claim 1, wherein R² is —O—C₁₋₆ alkyl or —S—C₁₋₆ alkyl and R³ is -halo.

23. The compound of claim 1, wherein R² is —O—C₁₋₆ alkyl or —S—C₁₋₆ alkyl and R³ is —F.

24. The compound of claim 1, wherein the compound is:

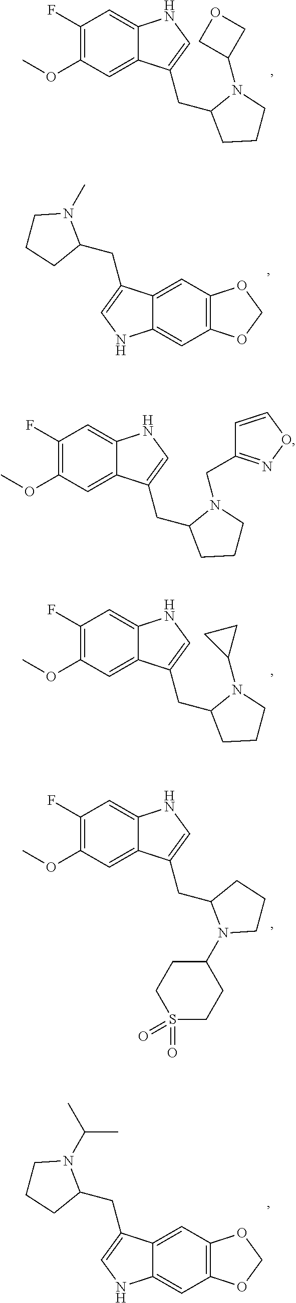 [SMILES: COc1cc2c(CC3CCCN3C3COC3)c[nH]c2cc1F ; CN1CCCC1Cc1c[nH]c2cc3c(cc12)OCO3 ; COc1cc2c(CC3CCCN3Cc3ccon3)c[nH]c2cc1F ; COc1cc2c(CC3CCCN3C3CC3)c[nH]c2cc1F ; COc1cc2c(CC3CCCN3C3CCS(=O)(=O)CC3)c[nH]c2cc1F ; CC(C)N1CCCC1Cc1c[nH]c2cc3c(cc12)OCO3]

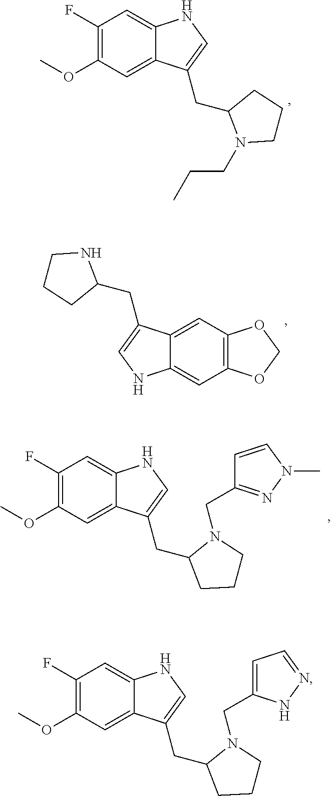

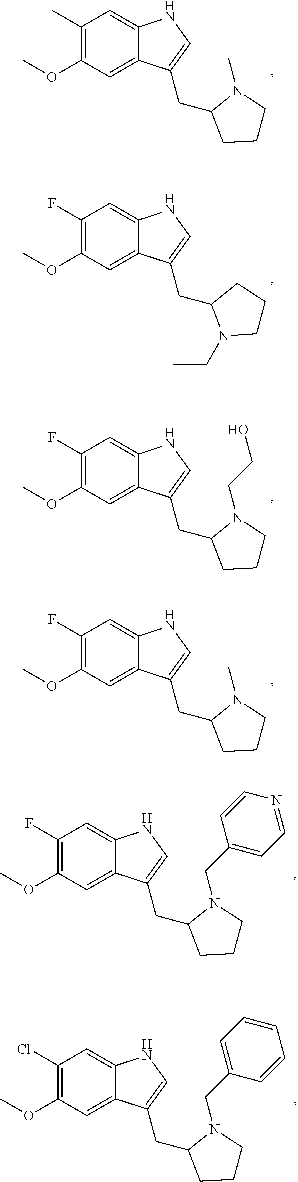

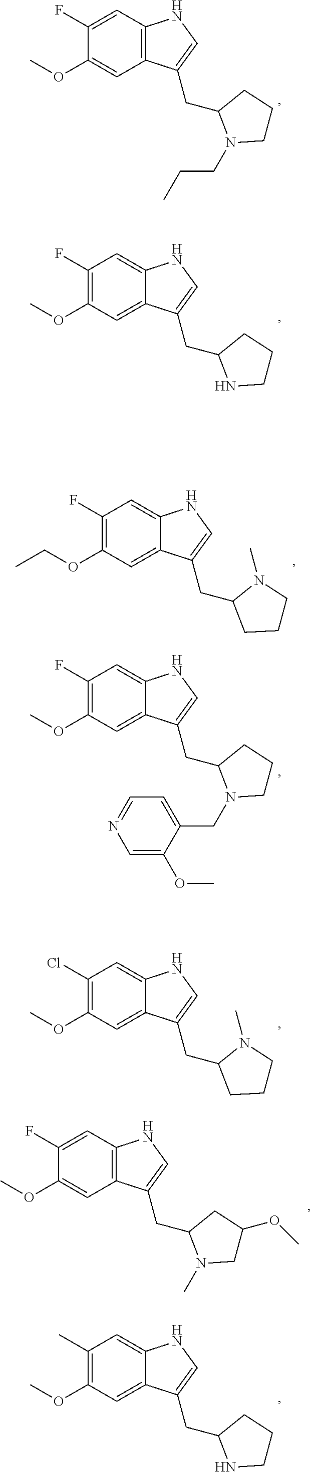

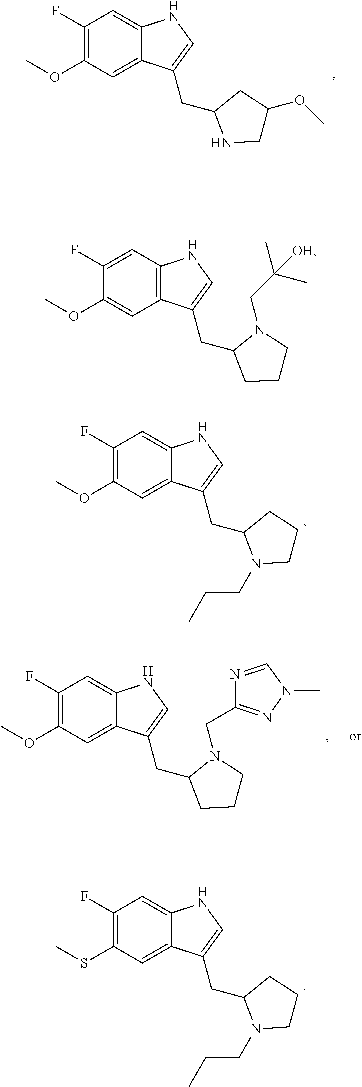

25. The compound of claim 1, wherein the compound is:

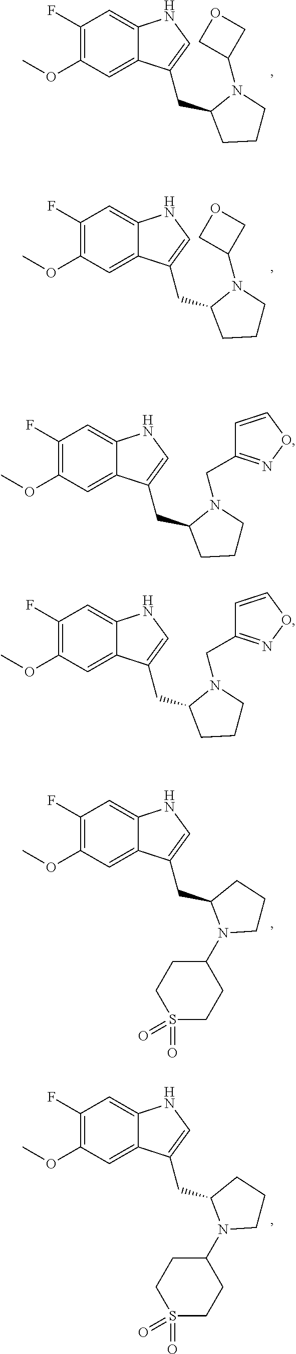 [SMILES: COc1cc2c(C[C@@H]3CCCN3C3COC3)c[nH]c2cc1F ; COc1cc2c(C[C@H]3CCCN3C3COC3)c[nH]c2cc1F ; COc1cc2c(C[C@@H]3CCCN3Cc3ccon3)c[nH]c2cc1F ; COc1cc2c(C[C@H]3CCCN3Cc3ccon3)c[nH]c2cc1F ; COc1cc2c(C[C@H]3CCCN3C3CCS(=O)(=O)CC3)c[nH]c2cc1F ; COc1cc2c(C[C@@H]3CCCN3C3CCS(=O)(=O)CC3)c[nH]c2cc1F]

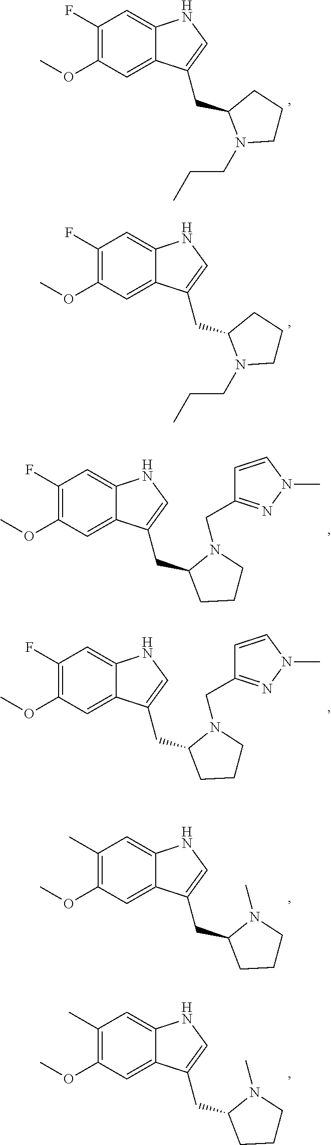

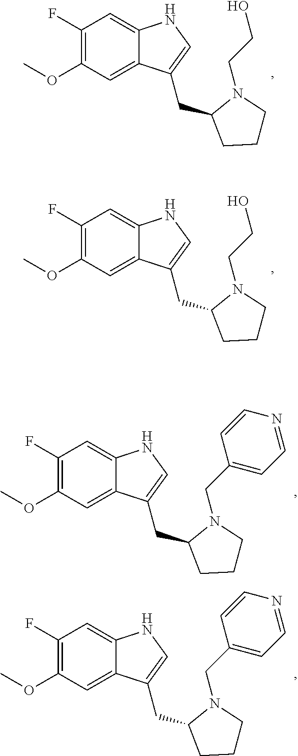

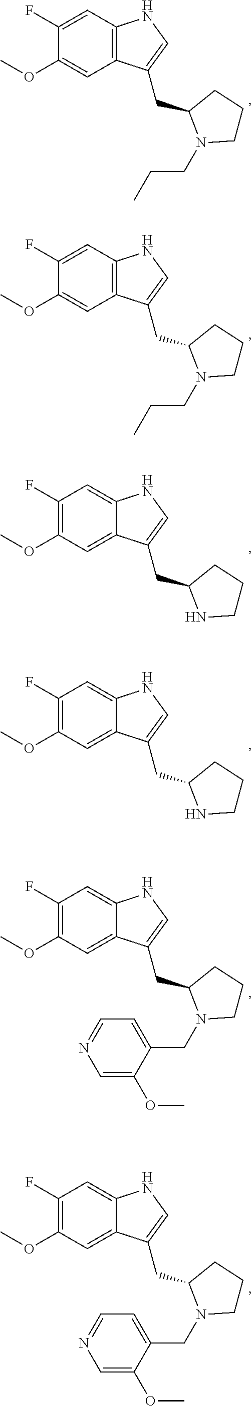 [SMILES: CCCN1CCC[C@@H]1Cc1c[nH]c2cc(F)c(OC)cc12 ; CCCN1CCC[C@H]1Cc1c[nH]c2cc(F)c(OC)cc12 ; COc1cc2c(C[C@H]3CCCN3)c[nH]c2cc1F ; COc1cc2c(C[C@@H]3CCCN3)c[nH]c2cc1F ; COc1cc2c(C[C@H]3CCCN3Cc3ccncc3OC)c[nH]c2cc1F ; COc1cc2c(C[C@@H]3CCCN3Cc3ccncc3OC)c[nH]c2cc1F]

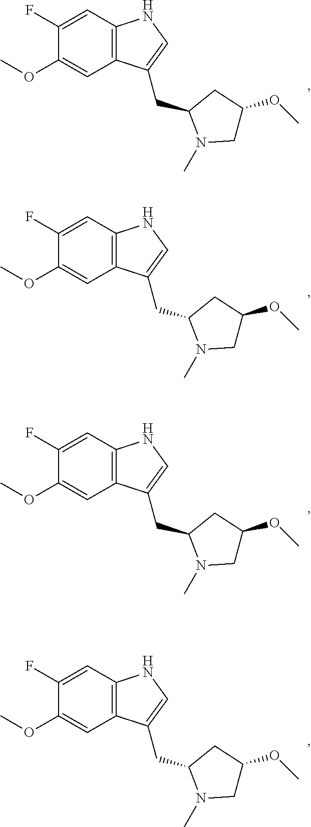

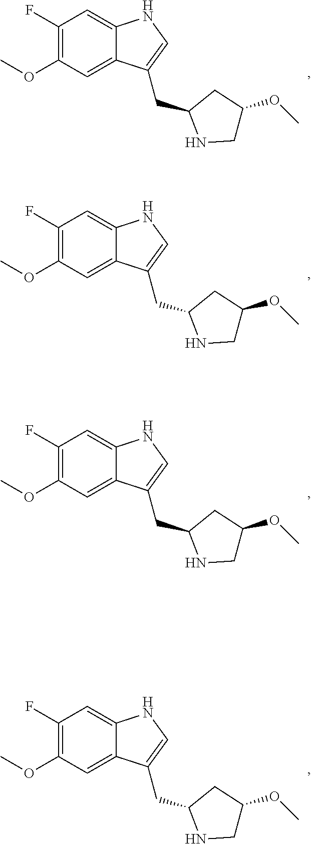

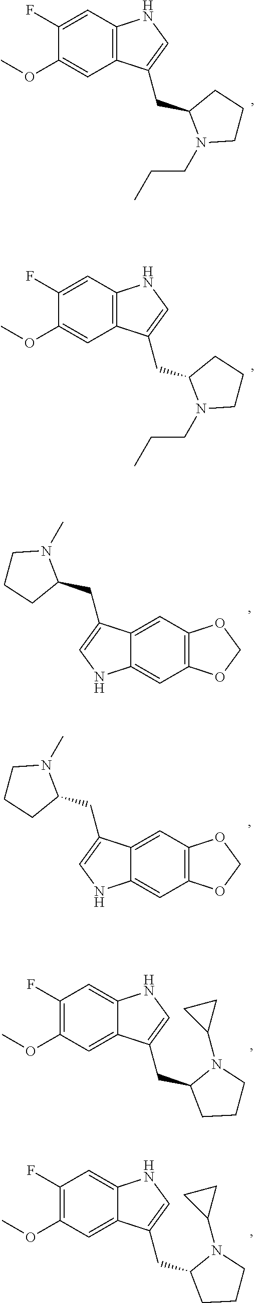 [SMILES: CCCN1CCC[C@@H]1Cc1c[nH]c2cc(F)c(OC)cc12 ; CCCN1CCC[C@H]1Cc1c[nH]c2cc(F)c(OC)cc12 ; CN1CCC[C@@H]1Cc1c[nH]c2cc3c(cc12)OCO3 ; CN1CCC[C@H]1Cc1c[nH]c2cc3c(cc12)OCO3 ; COc1cc2c(C[C@@H]3CCCN3C3CC3)c[nH]c2cc1F ; COc1cc2c(C[C@H]3CCCN3C3CC3)c[nH]c2cc1F]

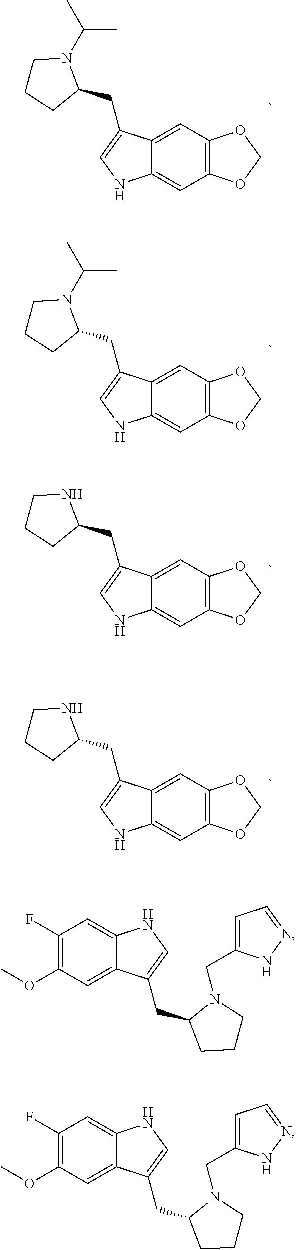 [SMILES: CC(C)N1CCC[C@@H]1Cc1c[nH]c2cc3c(cc12)OCO3 ; CC(C)N1CCC[C@H]1Cc1c[nH]c2cc3c(cc12)OCO3 ; c1[nH]c2cc3c(cc2c1C[C@H]1CCCN1)OCO3 ; c1[nH]c2cc3c(cc2c1C[C@@H]1CCCN1)OCO3 ; COc1cc2c(C[C@@H]3CCCN3Cc3ccn[nH]3)c[nH]c2cc1F ; COc1cc2c(C[C@H]3CCCN3Cc3ccn[nH]3)c[nH]c2cc1F]

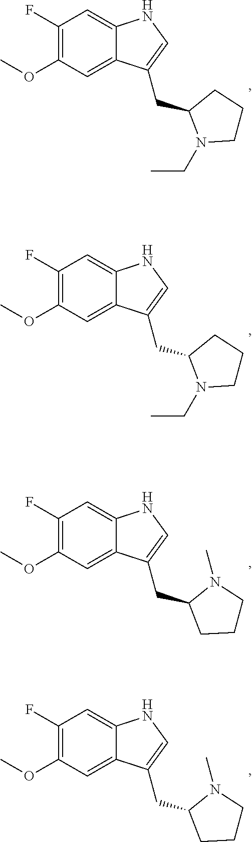 [SMILES: CCN1CCC[C@@H]1Cc1c[nH]c2cc(F)c(OC)cc12 ; CCN1CCC[C@H]1Cc1c[nH]c2cc(F)c(OC)cc12 ; COc1cc2c(C[C@@H]3CCCN3C)c[nH]c2cc1F ; COc1cc2c(C[C@H]3CCCN3C)c[nH]c2cc1F]

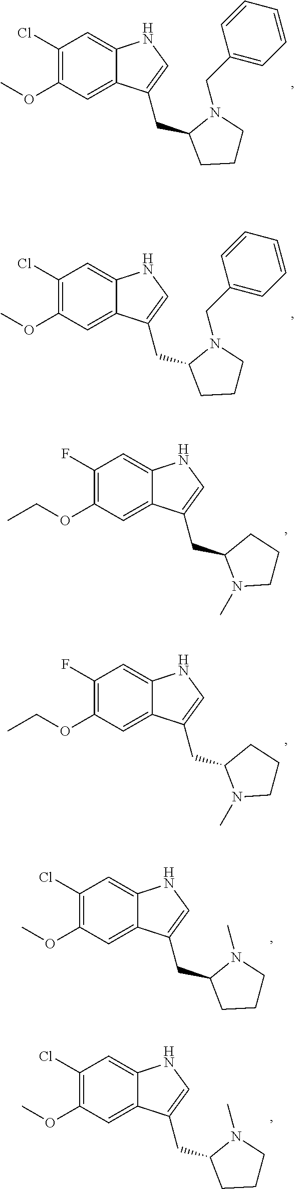 [SMILES: COc1cc2c(C[C@@H]3CCCN3Cc3ccccc3)c[nH]c2cc1Cl ; COc1cc2c(C[C@H]3CCCN3Cc3ccccc3)c[nH]c2cc1Cl ; CCOc1cc2c(C[C@H]3CCCN3C)c[nH]c2cc1F ; CCOc1cc2c(C[C@@H]3CCCN3C)c[nH]c2cc1F ; COc1cc2c(C[C@@H]3CCCN3C)c[nH]c2cc1Cl ; COc1cc2c(C[C@H]3CCCN3C)c[nH]c2cc1Cl]

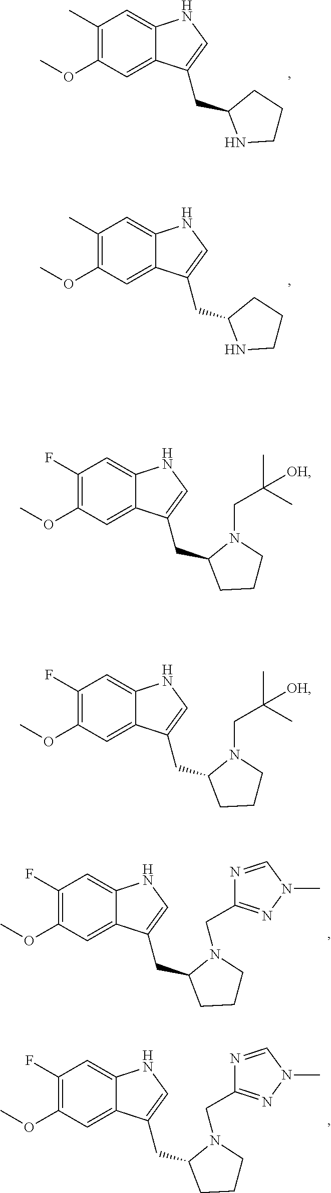

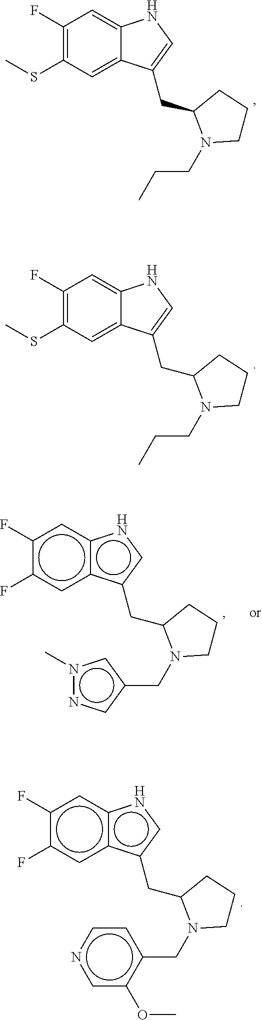

26. The compound of claim 1, wherein the compound is:

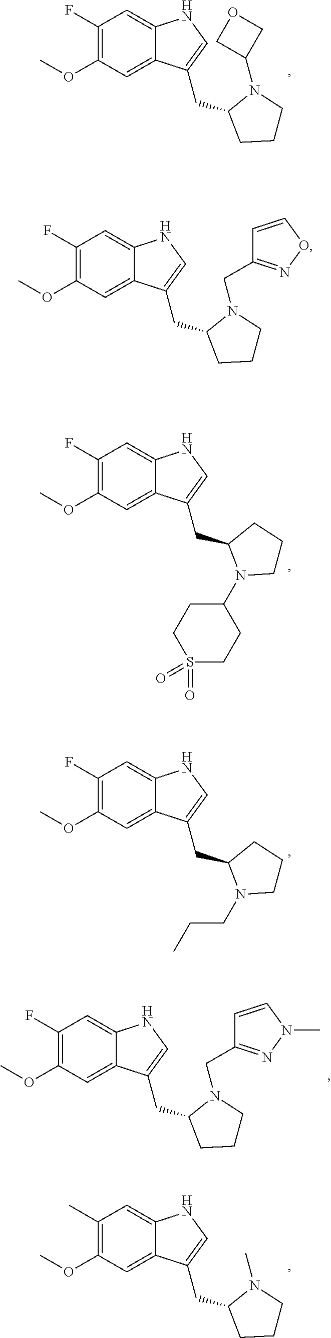

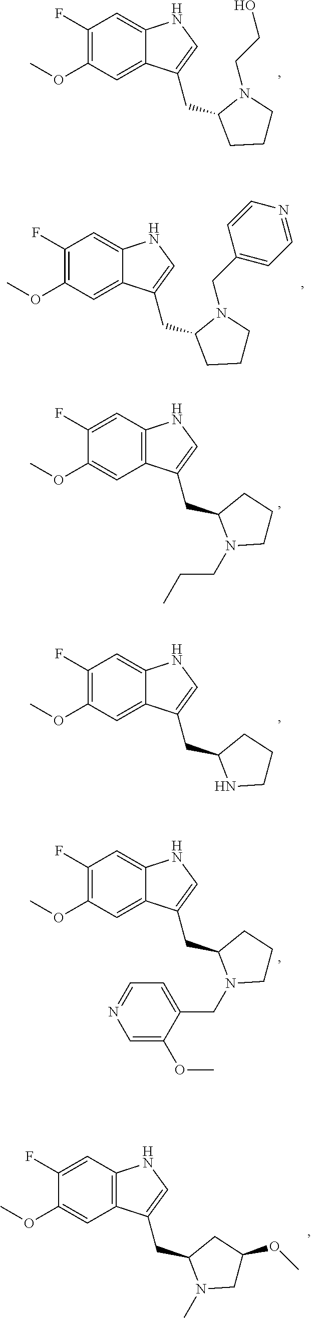

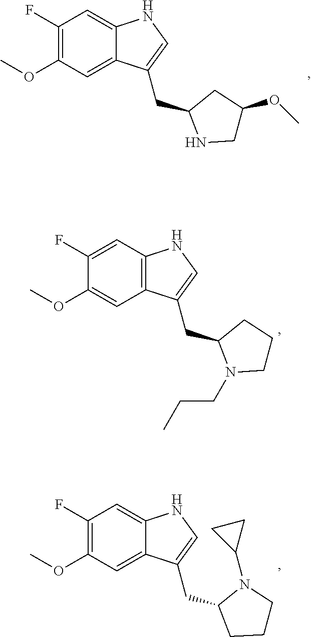

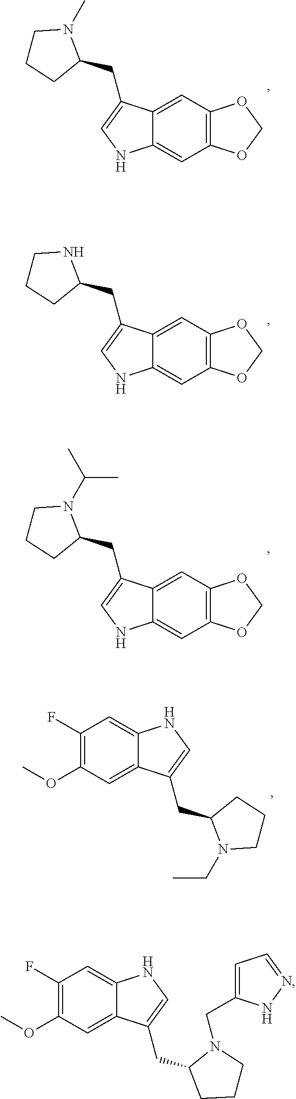 [SMILES: CN1CCC[C@@H]1Cc1c[nH]c2cc3c(cc12)OCO3 ; c1[nH]c2cc3c(cc2c1C[C@H]1CCCN1)OCO3 ; CC(C)N1CCC[C@@H]1Cc1c[nH]c2cc3c(cc12)OCO3 ; CCN1CCC[C@@H]1Cc1c[nH]c2cc(F)c(OC)cc12 ; COc1cc2c(C[C@H]3CCCN3Cc3ccn[nH]3)c[nH]c2cc1F]

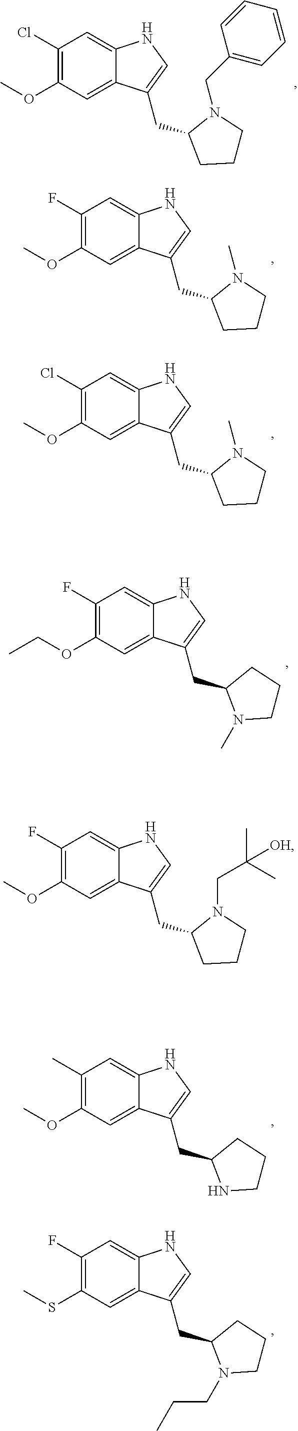

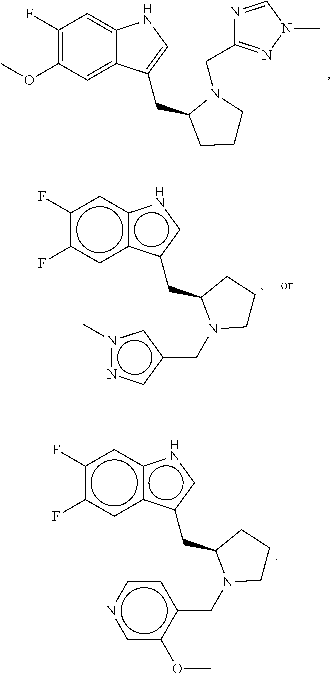

27. The compound of claim 1, wherein the compound is:

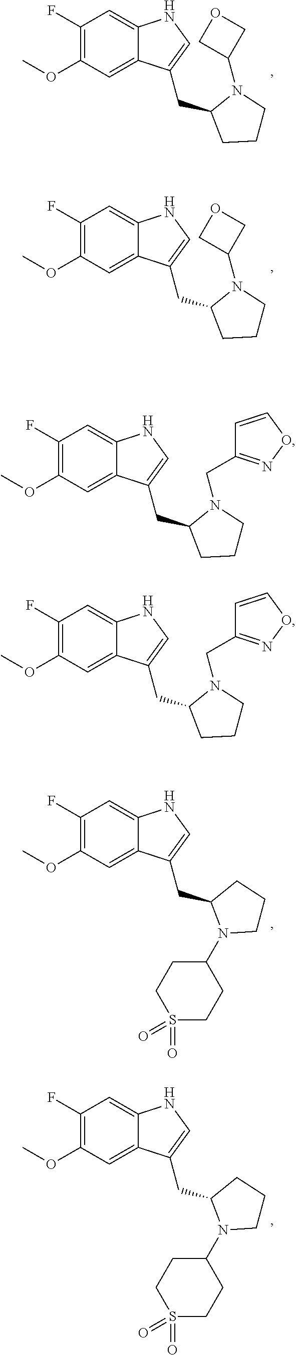 [SMILES: COc1cc2c(C[C@@H]3CCCN3C3COC3)c[nH]c2cc1F ; COc1cc2c(C[C@H]3CCCN3C3COC3)c[nH]c2cc1F ; COc1cc2c(C[C@@H]3CCCN3Cc3ccon3)c[nH]c2cc1F ; COc1cc2c(C[C@H]3CCCN3Cc3ccon3)c[nH]c2cc1F ; COc1cc2c(C[C@H]3CCCN3C3CCS(=O)(=O)CC3)c[nH]c2cc1F ; COc1cc2c(C[C@@H]3CCCN3C3CCS(=O)(=O)CC3)c[nH]c2cc1F]

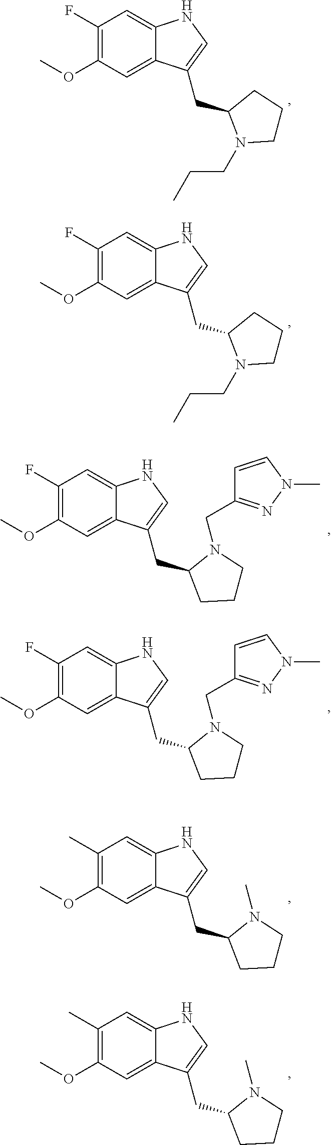

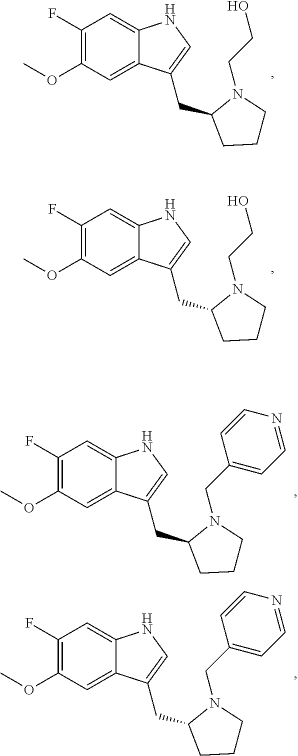

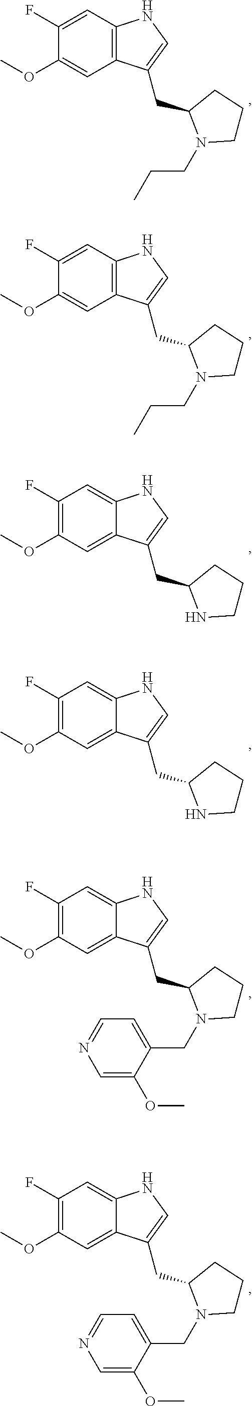 [SMILES: CCCN1CCC[C@@H]1Cc1c[nH]c2cc(F)c(OC)cc12 ; CCCN1CCC[C@H]1Cc1c[nH]c2cc(F)c(OC)cc12 ; COc1cc2c(C[C@H]3CCCN3)c[nH]c2cc1F ; COc1cc2c(C[C@@H]3CCCN3)c[nH]c2cc1F ; COc1cc2c(C[C@H]3CCCN3Cc3ccncc3OC)c[nH]c2cc1F ; COc1cc2c(C[C@@H]3CCCN3Cc3ccncc3OC)c[nH]c2cc1F]

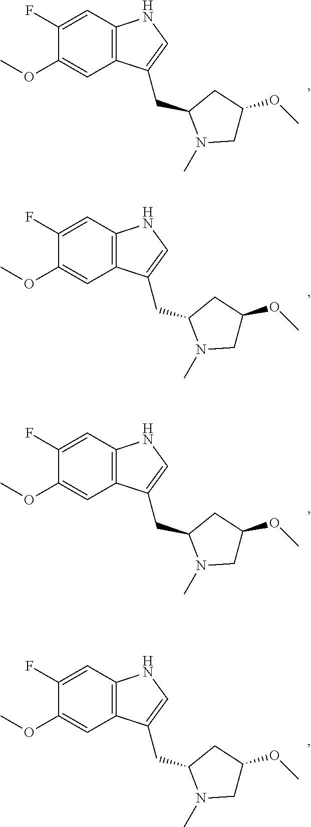

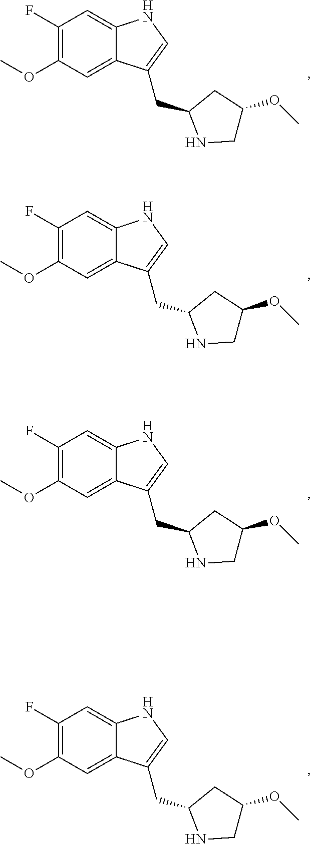

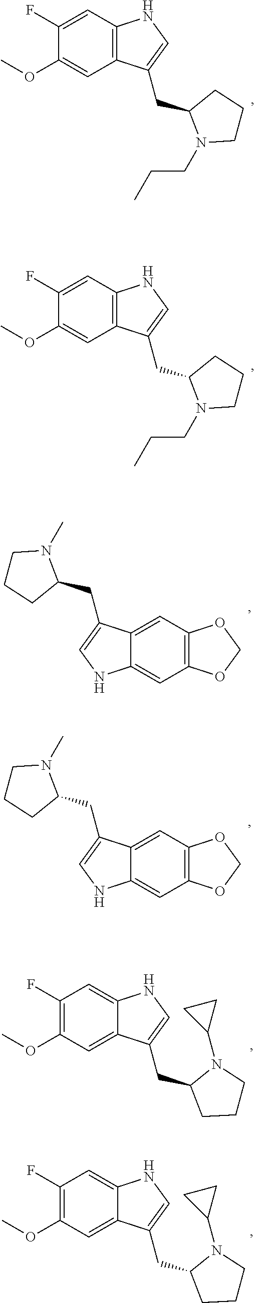 [SMILES: CCCN1CCC[C@@H]1Cc1c[nH]c2cc(F)c(OC)cc12 ; CCCN1CCC[C@H]1Cc1c[nH]c2cc(F)c(OC)cc12 ; CN1CCC[C@@H]1Cc1c[nH]c2cc3c(cc12)OCO3 ; CN1CCC[C@H]1Cc1c[nH]c2cc3c(cc12)OCO3 ; COc1cc2c(C[C@@H]3CCCN3C3CC3)c[nH]c2cc1F ; COc1cc2c(C[C@H]3CCCN3C3CC3)c[nH]c2cc1F]

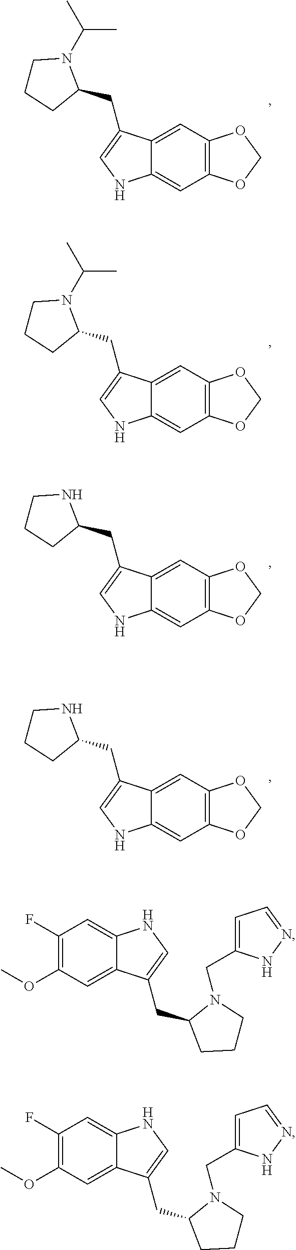 [SMILES: CC(C)N1CCC[C@@H]1Cc1c[nH]c2cc3c(cc12)OCO3 ; CC(C)N1CCC[C@H]1Cc1c[nH]c2cc3c(cc12)OCO3 ; c1[nH]c2cc3c(cc2c1C[C@H]1CCCN1)OCO3 ; c1[nH]c2cc3c(cc2c1C[C@@H]1CCCN1)OCO3 ; COc1cc2c(C[C@@H]3CCCN3Cc3ccn[nH]3)c[nH]c2cc1F ; COc1cc2c(C[C@H]3CCCN3Cc3ccn[nH]3)c[nH]c2cc1F]

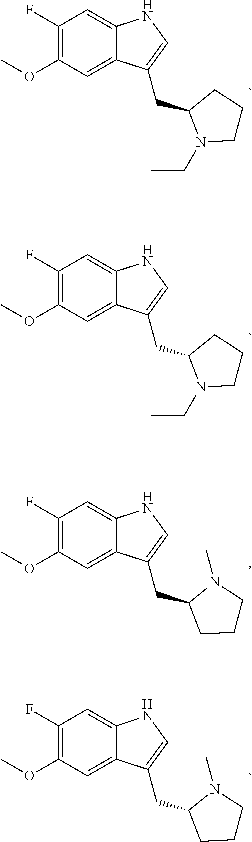 [SMILES: CCN1CCC[C@@H]1Cc1c[nH]c2cc(F)c(OC)cc12 ; CCN1CCC[C@H]1Cc1c[nH]c2cc(F)c(OC)cc12 ; COc1cc2c(C[C@@H]3CCCN3C)c[nH]c2cc1F ; COc1cc2c(C[C@H]3CCCN3C)c[nH]c2cc1F]

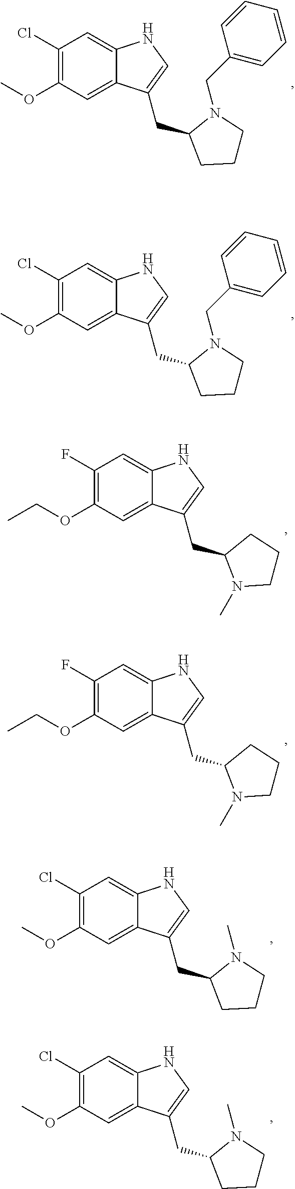 [SMILES: COc1cc2c(C[C@@H]3CCCN3Cc3ccccc3)c[nH]c2cc1Cl ; COc1cc2c(C[C@H]3CCCN3Cc3ccccc3)c[nH]c2cc1Cl ; CCOc1cc2c(C[C@H]3CCCN3C)c[nH]c2cc1F ; CCOc1cc2c(C[C@@H]3CCCN3C)c[nH]c2cc1F ; COc1cc2c(C[C@@H]3CCCN3C)c[nH]c2cc1Cl ; COc1cc2c(C[C@H]3CCCN3C)c[nH]c2cc1Cl]

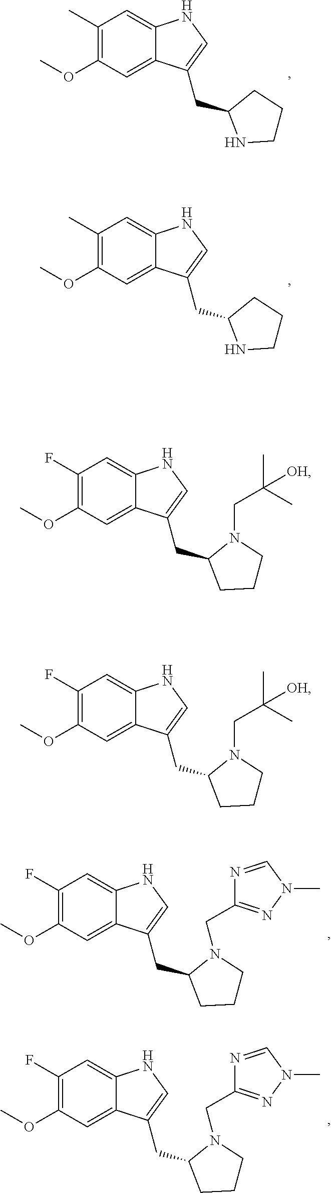

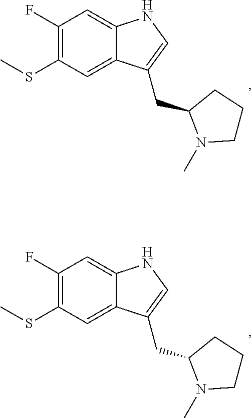 [SMILES: CSc1cc2c(C[C@H]3CCCN3C)c[nH]c2cc1F ; CSc1cc2c(C[C@@H]3CCCN3C)c[nH]c2cc1F]

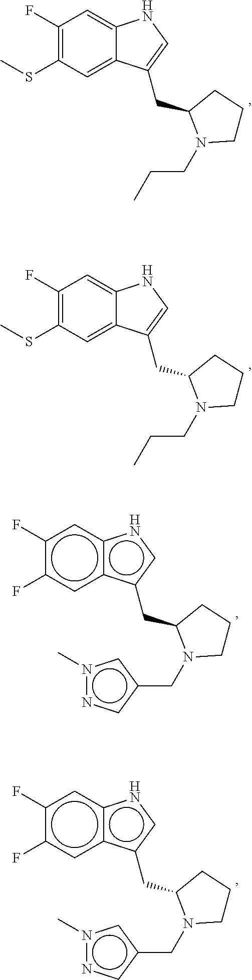

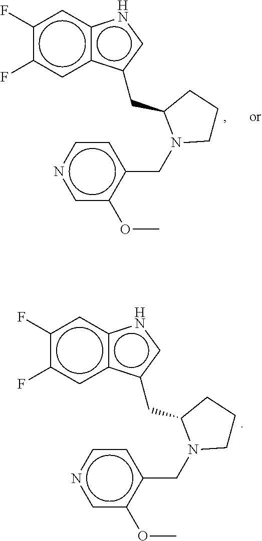

28. A pharmaceutical composition comprising a compound of claim 1, or a pharmaceutically acceptable salt or deuterated form thereof.

29. The pharmaceutical composition of claim 28, wherein the pharmaceutical composition is formulated for oral or parenteral administration.

30. The pharmaceutical composition of claim 29, wherein the parenteral administration is intravenous administration or subcutaneous administration.

31. A method of treating a 5-HT2A receptor associated disease or disorder, comprising administering a compound of claim 1, or a pharmaceutically acceptable salt or deuterated form thereof.

32. The method of claim 31, wherein the 5-HT2A receptor associated disease or disorder is an anxiety disorder, attention deficit hyperactivity disorder (ADHD), depression, cluster headache, diminished drive, burn-out, bore-out, migraine, Parkinson's disease, an eating disorder, psychotic disorder, schizophrenia, schizophreniform disorder, schizoaffective disorder, bipolar I disorder, bipolar II disorder, major depressive disorder, psychotic depression, delusional disorders, shared psychotic disorder, Shared paranoia disorder, brief psychotic disorder, paranoid personality disorder, schizoid personality disorder, schizotypal personality disorder, social anxiety disorder, substance-induced anxiety disorder, selective mutism, panic disorder, panic attacks, agoraphobia, posttraumatic stress disorder (PTSD), premenstrual dysphoric disorder (PMDD), or premenstrual syndrome (PMS).

33. The method of claim 31, wherein the 5-HT2A receptor associated disease or disorder is depression.

34. The method of claim 33, wherein the depression is treatment resistant depression.

35. The method of claim 31, wherein the 5-HT2A receptor associated disease or disorder is an eating disorder.

36. The method of claim 35, wherein the eating disorder is anorexia nervosa.

37. The method of claim 31, wherein the 5-HT2A receptor associated disease or disorder is an anxiety disorder.

38. The method of claim 31, wherein the 5-HT2A receptor associated disease or disorder is bipolar I disorder.

39. The method of claim 31, wherein the 5-HT2A receptor associated disease or disorder is bipolar II disorder.

40. The method of claim 31, wherein the 5-HT2A receptor associated disease or disorder is major depressive disorder.

41. The method of claim 31, wherein the 5-HT2A receptor associated disease or disorder is posttraumatic stress disorder (PTSD).
